(19) United States
(12) Patent Application Publication
(10) Pub. No.: US 2022/0387515 A1
(43) Pub. Date: Dec. 8, 2022

(54) DIFFERENTIAL KNOCKOUT OF AN ALLELE OF A HETEROZYGOUS ELANE GENE USING GUIDES 21-30 NUCLEOTIDES IN LENGTH

(71) Applicants: David Baram, Tel Aviv (IL); Lior Izhar, Tel Aviv (IL); Asael Herman, Ness-Ziona (IL); Rafi Emmanuel, Ramla (IL); Liat Rockah, Rishon LeZion (IL); Nadav Marbach Bar, Rehovot (IL); Michal Golan Mashiach, Ness-Ziona (IL); Joseph Georgeson, Rehovot (IL)

(72) Inventors: David Baram, Tel Aviv (IL); Lior Izhar, Tel Aviv (IL); Asael Herman, Ness-Ziona (IL); Rafi Emmanuel, Ramla (IL); Liat Rockah, Rishon LeZion (IL); Nadav Marbach Bar, Rehovot (IL); Michal Golan Mashiach, Ness-Ziona (IL); Joseph Georgeson, Rehovot (IL)

(73) Assignee: EmendoBio Inc., Wilmington, DE (US)

(21) Appl. No.: 17/775,017

(22) PCT Filed: Nov. 5, 2020
(86) PCT No.: PCT/US2020/059186

§ 371 Date: May 6, 2022

Related U.S. Application Data
(60) Provisional application No. 62/931,659, filed on Nov. 6, 2019.

(51) Int. Cl. A61K 35/32; C12N 15/11; C12N 9/22; C12N 15/90; C12N 5/0789; A61K 35/17; A61P 7/00

(52) U.S. Cl. CPC A61K 35/32; C12N 15/11; C12N 9/22; C12N 15/907; C12N 5/0647; A61K 35/17; A61P 7/00; C12N 2310/20; C12N 2800/80

(57) ABSTRACT

Methods for inactivating in a cell a mutant allele of the elastase, neutrophil expressed gene (ELANE gene) gene having a mutation associated with severe congenital neutropenia (SCN) or cyclic neutropenia (CyN) and which cell is heterozygous at one or more polymorphic sites selected from the group consisting of: rs10424470, rs4807932, rs10414837, rs376107533, rs3761010, rs351108, rs3826946, rs10413889, rs3761007, rs10409474, rs3761005, rs351107, rs3761001, rs740021, rs781452480, rs371057361, rs570466264, rs1041904080, rs7250194, rs17216649, rs199720952, rs6510983, rs17223066, rs7255385, rs9749274, rs111361200, rs112639467, rs141213775, rs28591229, rs10469327, rs3834645, rs1683564, rs71335276, and rs8107095, the method comprising
introducing to the cell a composition comprising:
   a CRISPR nuclease or a sequence encoding the CRISPR nuclease; and
   a first RNA molecule comprising a guide sequence portion having 21-30 nucleotides,
wherein a complex of the CRISPR nuclease and the first RNA molecule affects a double strand break in the mutant allele of the ELANE gene.

This application claims the benefit of U.S. Provisional Application No. 62/931,659 filed Nov. 6, 2019, the contents of which are hereby incorporated by reference.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### REFERENCE TO SEQUENCE LISTING

This application incorporates-by-reference nucleotide sequences which are present in the file named “201105_91193-A-PCT_Sequence_Listing_AWG.txt”, which is 779 kilobytes in size, and which was created on Nov. 5, 2020 in the IBM-PC machine format, having an operating system compatibility with MS-Windows, which is contained in the text file filed Nov. 5, 2020 as part of this application.

### BACKGROUND OF INVENTION

There are several classes of DNA variation in the human genome, including insertions and deletions, differences in the copy number of repeated sequences, and single nucleotide polymorphisms (SNPs). A SNP is a DNA sequence variation occurring when a single nucleotide (adenine (A), thymine (T), cytosine (C), or guanine (G)) in the genome differs between human subjects or paired chromosomes in an individual. Over the years, the different types of DNA variations have been the focus of the research community either as markers in studies to pinpoint traits or disease causation or as potential causes of genetic disorders. SNPs are usually considered benign and not causing disease.

A genetic disorder is caused by one or more abnormalities in the genome. Genetic disorders may be regarded as either “dominant” or “recessive.” Recessive genetic disorders are those which require two copies (i.e., two alleles) of the abnormal/defective gene to be present. In contrast, a dominant genetic disorder involves a gene or genes which exhibit(s) dominance over a normal (functional/healthy) gene or genes. As such, in dominant genetic disorders only a single copy (i.e., allele) of an abnormal gene is required to cause or contribute to the symptoms of a particular genetic disorder. Such mutations include, for example, gain-of-function mutations in which the altered gene product possesses a new molecular function or a new pattern of gene expression. Gain-of-function mutations are generally dominant negative mutations. An example of a dominant negative mutation is haploinsufficiency where one allele is mutated and loses its function and the single wild type allele left does not generate enough protein to be sufficient for a specific cellular function. Other examples include dominant negative mutations which have a gene product that acts antagonistically to the wild-type allele.

### Neutropenia

Neutropenia is defined as a reduction in the absolute number of neutrophils in the blood circulation and commonly diagnosed by measuring the absolute neutrophil count (ANC) in peripheral blood. The severity of neutropenia is characterized as mild with an ANC of 1000-1500/4, moderate with an ANC of 500-1000/4, or severe with an ANC of less than 500/4 (Boxer 2012).

Neutropenia can be classified as congenital (hereditary) or acquired. The two main types of the congenital condition, commonly of autosomal dominant inheritance, are cyclic neutropenia (CyN) and severe congenital neutropenia (SCN). Cyclic neutropenia is characterized by fluctuating neutrophil counts from normal levels to zero while severe congenital neutropenia (SCN) is characterized by very low ANC (500/4) observed at birth, maturation arrest of the myelopoiesis in bone marrow at the promyelocyte/myelocyte stage, and early onset of bacterial infections (Carlsson et al. 2012; Horwitz et al. 2013).

SCN may be diagnosed by measuring a very low ANC in the blood and examining bone marrow aspirate to identify myeloid maturation arrest (Dale 2017). SCN is usually diagnosed before age 6 months, while diagnosis for CyN is generally raised during the second year of life, or later, and the main clinical manifestation is recurrent acute stomatologic disorders. Bone marrow examination is often necessary to rule out malignant hemopathies, determine cellularity, assess myeloid maturation, and detect signs of a precise etiology, with cytogenetic bone marrow studies now crucial when SCN is suspected. Antineutrophil antibody assay, immunoglobulin assay (Ig GAM), lymphocyte immunophenotyping, pancreatic markers (serum trypsinogen and fecal elastase) and liposoluble vitamin levels (vitamins A, E and D) are also of interest in assessing SCN and CyN (See Donadieu 2011).

SCN can be autosomal-recessive (HAX1, G6PC3), autosomal-dominant (ELANE, GFI1), or X-linked (WAS) forms of inheritance or occur sporadically (Carlsson et al. 2012; Boxer 2012).

Cyclic and congenital neutropenia are most frequently caused by mutations in the “elastase, neutrophil expressed gene” (ELANE gene)—the gene for neutrophil elastase. “ELANE gene mutations are identified in 40-55% of SCN patients and males and females are equally affected (Donadieu et al. 2011; Dale 2017). Mutations in the ELANE gene are associated with autosomal-dominant and sporadic cases of SCN (Carlsson et al. 2012). To date, more than 200 different ELANE mutations have been identified, which are randomly distributed over all exons as well as in intron 3 and intron 4 (Skokowa et al. 2017). More than 120 distinct ELANE gene mutations related to CyN and SCN are now known, for example C151Y and G214R particularly associated with a poor prognosis. (See Makaryan et al. 2012; see also Germeshausen et al. 2013 for a comprehensive list of ELANE mutations related to CyN and SCN).

ELANE encodes neutrophil elastase (NE) which is involved in the function of neutrophil extracellular traps (networks of fibers that bind pathogens). Some studies suggest that the product of mutant ELANE acts to disrupt neutrophil production in the bone marrow and cause neutropenia. These studies indicate that mutations in NE initiate the unfolded protein response (UPR) leading to cell loss in the process of neutrophil formation in the marrow (Makaryan et al. 2017).

### Current Treatments

Granulocyte colony-stimulating factor (G-CSF) is considered the first-line treatment for SCN (Connelly, Choi, and Levine 2012). G-CSF stimulates the production of more neutrophils and delays their apoptosis (Schäffer and Klein 2007). Overall survival is now estimated to exceed 80%, including patients developing malignancies, although 10% of SCN patients still die from severe bacterial infections or sepsis (Skokowa et al. 2017). Although G-CSF therapy is successful in preventing mortality from sepsis, long-term treatment was identified to be associated with an increased risk of developing myelodysplastic syndrome (MDS) or leukemia in SCN patients. The most common leukemia in SCN is AML, but acute lymphoid leukemia (ALL), juvenile myelomonocytic leukemia (JMML), chronic myelomonocytic leukemia (CMML), and bi-phenotypic leukemia are also reported in the literature (Connelly, Choi, and Levine 2012). It was previously demonstrated that patients who had a robust response to G-CSF (doses≤8 μg/kg/day) had a cumulative incidence of 15% for developing MDS/leukemia after 15 years on G-CSF, while an incidence of 34% was reported in patients with poor response to G-CSF despite high doses (Rosenberg et al. 2010).

Hematopoietic stem cell transplant (HSCT) is an alternative, curative therapy for patients who do not respond to G-CSF therapy or who develop AML/MDS. However, patients with chronic neutropenia who undergo HCT are at increased risk of developing infectious complications such as fungal and graft-versus-host disease (Skokowa et al. 2017). Moreover, HCT requires a matched related donor for successful survival but most patients will not have an available matched donor (Connelly, Choi, and Levine 2012).

### SUMMARY OF THE INVENTION

Disclosed is an approach for knocking out the expression of a dominant-mutant allele by disrupting the dominant-mutant allele or degrading the resulting mRNA.

The present disclosure provides a method for utilizing at least one naturally occurring heterozygous nucleotide difference or polymorphism (e.g., single nucleotide polymorphism (SNP)) for distinguishing/discriminating between two alleles of a gene, one allele bearing a mutation such that it encodes a mutated protein causing a disease phenotype (“mutant allele”), and the other allele encoding for a functional protein (“functional allele”).

Embodiments of the present invention provide methods for utilizing at least one heterozygous SNP in a gene expressing a dominant mutant allele in a given cell or subject. In embodiments of the present invention, the SNP utilized may or may not be associated with a disease phenotype. In embodiments of the present invention, an RNA molecule comprising a guide sequence targets the mutant allele of the gene by targeting the nucleotide base present at a heterozygous SNP in the mutant allele of the gene and therefore having a different nucleotide base from the functional allele of the gene.

In some embodiments, the method further comprises the step of knocking out expression of the mutated protein and allowing expression of the functional protein.

The present invention provides a method for inactivating in a cell a mutant allele of the elastase, neutrophil expressed gene (ELANE gene) gene having a mutation associated with severe congenital neutropenia (SCN) or cyclic neutropenia (CyN) and which cell is heterozygous at one or more polymorphic sites selected from the group consisting of: rs10424470, rs4807932, rs10414837, rs376107533, rs3761010, rs351108, rs3826946, rs10413889, rs3761007, rs10409474, rs3761005, rs351107, rs3761001, rs740021, rs781452480, rs371057361, rs570466264, rs1041904080, rs7250194, rs17216649, rs199720952, rs6510983, rs17223066, rs7255385, rs9749274, rs111361200, rs112639467, rs141213775, rs28591229, rs10469327, rs3834645, rs1683564, rs71335276, and rs8107095 the method comprising
introducing to the cell a composition comprising:
   a CRISPR nuclease or a sequence encoding the CRISPR nuclease; and
   a first RNA molecule comprising a guide sequence portion having 21-30 nucleotides,
wherein a complex of the CRISPR nuclease and the first RNA molecule affects a double strand break in the mutant allele of the ELANE gene.

The present invention provides for a modified cell obtained by the methods of the present invention.

The present invention provides for a modified cell lacking at least a portion of one allele of the ELANE gene.

The present invention provides for a composition comprising modified cells and a pharmaceutically acceptable carrier.

The present invention provides for an in vitro or ex vivo method of preparing a composition, comprising mixing the cells of the present invention with the pharmaceutically acceptable carrier.

The present invention provides for a method of preparing in vitro or ex vivo a composition comprising modified cells, the method comprising:
a) isolating HSPCs from cells obtained from a subject with an ELANE gene mutation related to SCN or CyN and/or suffering from SCN or CyN and which subject is heterozygous at one or more polymorphic sites selected from the group consisting of: rs10424470, rs4807932, rs10414837, rs376107533, rs3761010, rs351108, rs3826946, rs10413889, rs3761007, rs10409474, rs3761005, rs351107, rs3761001, rs740021, rs781452480, rs371057361, rs570466264, rs1041904080, rs7250194, rs17216649, rs199720952, rs6510983, rs17223066, rs7255385, rs9749274, rs111361200, rs112639467, rs141213775, rs28591229, rs10469327, rs3834645, rs1683564, rs71335276, and rs8107095, and obtaining the cell from the subject;
b) introducing to the cells of step (a) a composition comprising:
   a CRISPR nuclease or a sequence encoding the CRISPR nuclease; and
   a first RNA molecule comprising a guide sequence portion having 21-30 nucleotides,
wherein a complex of the CRISPR nuclease and the first RNA molecule affects a double strand break in the mutant allele of the ELANE gene in one or more cells,
   optionally, introducing to the cells a second RNA molecule comprising a guide sequence portion capable of complexing with a CRISPR nuclease, wherein the complex of the second RNA molecule and CRISPR nuclease affects a second double strand break in the ELANE gene in the one or more cells
so as to inactive the mutant allele of the ELANE gene in one or more cells thereby obtaining modified cells; optionally
c) culture expanding the modified cells of step (b),
wherein the modified cells are capable of engraftment and giving rise to progeny cells after engraftment.

The present invention provides for use of a composition prepared in vitro by a method comprising:
a) isolating HSPCs from cells obtained from a subject with an ELANE gene mutation related to SCN or CyN and/or suffering from SCN or CyN and which subject is heterozygous at one or more polymorphic sites selected from the group consisting of: rs10424470, rs4807932, rs10414837, rs376107533, rs3761010, rs351108, rs3826946, rs10413889, rs3761007, rs10409474, rs3761005, rs351107, rs3761001, rs740021, rs781452480, rs371057361, rs570466264, rs1041904080, rs7250194, rs17216649, rs199720952, rs6510983, rs17223066, rs7255385, rs9749274, rs111361200, rs112639467, rs141213775, rs28591229, rs10469327, rs3834645, rs1683564, rs71335276, and rs8107095;
b) introducing to the cells of step (a) a composition comprising:
   a CRISPR nuclease or a sequence encoding the CRISPR nuclease; and
   a first RNA molecule comprising a guide sequence portion having 21-30 nucleotides,
wherein a complex of the CRISPR nuclease and the first RNA molecule affects a double strand break in the mutant allele of the ELANE gene in one or more cells,
   optionally, introducing to the cells a second RNA molecule comprising a guide sequence portion capable of complexing with a CRISPR nuclease, wherein the complex of the second RNA molecule and CRISPR nuclease affects a second double strand break in the ELANE gene in the one or more cells
so as to inactive the mutant allele of the ELANE gene in one or more cells thereby obtaining modified cells; optionally;
c) culture expanding the cells of step (b) wherein the modified cells are capable of engraftment and giving rise to progeny cells after engraftment; and
d) administering to the subject the cells of step (b) or step (c) for treating the SCN or CyN in the subject.

The present invention provides for a method of treating a subject afflicted with SCN or CyN, comprising administration of a therapeutically effective amount of the modified cells, compositions, or the compositions prepared by the methods of the instant invention

The present invention provides for a method for treating SCN or CyN in a subject with an ELANE gene mutation relating to SCN or CYN in need thereof and which subject is heterozygous at one or more polymorphic sites selected from the group consisting of: rs10424470, rs4807932, rs10414837, rs376107533, rs3761010, rs351108, rs3826946, rs10413889, rs3761007, rs10409474, rs3761005, rs351107, rs3761001, rs740021, rs781452480, rs371057361, rs570466264, rs1041904080, rs7250194, rs17216649, rs199720952, rs6510983, rs17223066, rs7255385, rs9749274, rs111361200, rs112639467, rs141213775, rs28591229, rs10469327, rs3834645, rs1683564, rs71335276, and rs8107095, the method comprising:
a) isolating HSPCs from cells obtained from the subject;
b) introducing to the cells of step (a) a composition comprising:
   a CRISPR nuclease or a sequence encoding the CRISPR nuclease; and
   a first RNA molecule comprising a guide sequence portion having 21-30 nucleotides,
wherein a complex of the CRISPR nuclease and the first RNA molecule affects a double strand break in the mutant allele of the ELANE gene in one or more cells,
   optionally, introducing to the cells a second RNA molecule comprising a guide sequence portion capable of complexing with a CRISPR nuclease, wherein the complex of the second RNA molecule and CRISPR nuclease affects a second double strand break in the ELANE gene in the one or more cells
so as to inactive the mutant allele of the ELANE gene in one or more cells thereby obtaining modified cells; optionally;
c) culture expanding the cells of step (b) wherein the modified cells are capable of engraftment and giving rise to progeny cells after engraftment; and
d) administering to the subject the cells of step (b) or step (c)
thereby treating the SCN or CyN in the subject.

The present invention provides for a method for treating SCN or CyN in a subject with an ELANE gene mutation relating to SCN or CYN in need thereof and which subject is heterozygous at one or more polymorphic sites selected from the group consisting of: rs10424470, rs4807932, rs10414837, rs376107533, rs3761010, rs351108, rs3826946, rs10413889, rs3761007, rs10409474, rs3761005, rs351107, rs3761001, rs740021, rs781452480, rs371057361, rs570466264, rs1041904080, rs7250194, rs17216649, rs199720952, rs6510983, rs17223066, rs7255385, rs9749274, rs111361200, rs112639467, rs141213775, rs28591229, rs10469327, rs3834645, rs1683564, rs71335276, and rs8107095, the method comprising
administering to the subject autologous modified cells or progeny of autologous modified cells, wherein the autologous modified cells are modified so as to have a double strand break in the mutant allele of the ELANE gene,
   wherein said double strand break results from introduction to the cells of a composition comprising a CRISPR nuclease or a sequence encoding the CRISPR nuclease and a first RNA molecule wherein a complex of the CRISPR nuclease and the first RNA molecule affects a double strand break in the mutant allele of the ELANE gene so as to inactive the mutant allele of the ELANE gene in the cell,
thereby treating the SCN or CyN in the subject.

The present invention provides for a method of selecting a subject for treatment from a pool of subjects diagnosed with SCN or CyN, comprising the steps of:
a) obtaining cells from each subject in the pool of subjects;
b) screening each subject's cells for an ELANE gene mutation related to SCN or CyN, and selecting only subjects with an ELANE gene mutation related to SCN or CyN;
c) screening by sequencing the cells of the subjects selected in step (b) for heterozygosity at one or more polymorphic sites selected from the group consisting of: rs10414837, rs3761005, rs1683564,
d) selecting for treatment only subjects with cells heterozygous at the one of more polymorphic sites
e) obtaining hematopoetic stem and progenitor cells (HSPC) cells from the bone marrow of the subject either by aspiration or by mobilization and apheresis of peripheral blood; and
f) introducing to the HSPC cells of step (e):
   one or more CRISPR nucleases or sequences encoding the one or more CRISPR nuclease;
   a first RNA molecule comprising a guide sequence portion having 21-30 contiguous nucleotides containing nucleotides in the sequence set forth in any one of SEQ ID NOs: 1-3751, targeting the nucleotide base of the heterozygous allele of the one or more polymorphic sites present on the mutant allele of the ELANE gene, and
   a second RNA molecule comprising a guide sequence portion targeting a sequence in intron 4 of the ELANE gene, optionally wherein the guide sequence portion of the second RNA molecule has 21-30 nucleotides, optionally wherein 21-30 contiguous nucleotides contain nucleotides in the sequence set forth in any one of SEQ ID NOs: 2954-3751.
wherein a complex of the first RNA molecule and a CRISPR nuclease affects a first double strand break in the mutant allele of the ELANE gene in one or more of the HSPC cells and a complex of the second RNA molecule and a CRISPR nuclease affect a second double strand break in intron 4 of both alleles of the ELANE gene in the one or more HSPC cells in which the complex of the first RNA molecule and the CRISPR nuclease affected a first double strand break, thereby obtaining modified cells;
g) administering to the subject the modified cells of step (0,
thereby treating SCN or CyN in the subject.

The present invention provides an RNA molecule comprising a guide sequence portion having 21-30 contiguous nucleotides containing nucleotides in the sequence set forth in any one of SEQ ID NOs: 1-3751.

The present invention provides a method for inactivating in a cell a mutant ELANE allele, the method comprising delivering to the cell the RNA molecules or compositions of the present invention.

The present invention provides use of the RNA molecules, the compositions, or the composition prepared by the method of the present invention for inactivating in a cell a mutant ELANE allele.

The present invention provides a medicament comprising the RNA molecules, compositions, or the compositions prepared by the methods of the instant invention for use in inactivating in a cell a mutant ELANE allele, wherein the medicament is administered by delivering to the cell the RNA molecules, compositions, or the compositions prepared by the methods of the instant invention.

The present invention provides for use of the methods, the modified cells, the compositions, or the compositions prepared by the methods, or the RNA molecules of the instant invention for treating ameliorating or preventing SCN or CyN in to a subject having or at risk of having SCN or CyN.

The present invention provides for a medicament comprising the RNA molecules, compositions, compositions prepared by the methods of the instant invention, or the modified cells of the instant invention, for use in treating ameliorating or preventing SCN or CyN, wherein the medicament is administered by delivering to a subject having or at risk of having SCN or CyN the RNA molecules, compositions, compositions prepared by the methods of the instant invention, or the modified cells of the instant invention.

The present invention provides for a kit for inactivating a mutant ELANE allele in a cell, comprising the RNA molecules of the instant invention, a CRISPR nuclease or a sequence encoding the CRISPR nuclease, and/or a tracrRNA molecule or a sequence encoding the tracrRNA; and instructions for delivering the RNA molecule; CRISPR nuclease or sequence encoding the CRISPR nuclease, and/or tracrRNA molecule or sequence encoding the tracrRNA to the cell to inactivate the mutant ELANE allele in the cell.

The present invention provides for a kit for treating SCN or CyN in a subject, comprising the RNA molecules of the instant invention, a CRISPR nuclease or sequence encoding the CRISPR nuclease, and/or a tracrRNA molecule or sequence encoding the tracrRNA molecule; and instructions for delivering the RNA molecule; CRISPR nuclease, and/or the tracrRNA to a subject having or at risk of having SCN or CyN so as to treat the SCN or CyN.

The present invention provides a kit for inactivating a mutant ELANE allele in a cell, comprising the compositions, the composition prepared by the methods of the instant invention, or the modified cells of the instant invention, and instructions for delivering the composition to the cell so as to inactivate the ELANE gene in the cell.

The present invention provides a kit for treating SCN or CyN in a subject, comprising the composition, the compositions prepared by the methods of the instant invention, or the modified cells of the instant invention, and instructions for delivering the compositions, the compositions prepared by the methods of the instant invention, or the modified cells of the instant invention, to a subject having or at risk of having SCN or CyN so as to treat SCN or CyN.

### BRIEF DESCRIPTION OF THE DRAWINGS

FIG. 1: Schematic showing excising the promoter region from an upstream SNP position until intron 3 or intron 4 or the 3′ UTR. In one example, a first guide sequence targets a specific sequence of a heterozygous SNP position in an upstream region of the mutant allele (strategy 1a—rs10414837, strategy 1b—rs3761005) and a second guide sequence targets a sequence in intron 4 which is common to two alleles of the gene.

FIG. 2: Schematic showing excising the promoter region from an upstream SNP position until intron 3 or intron 4 or the 3′ UTR. In one example, a first guide sequence targets a specific sequence of a heterozygous SNP position in an upstream region of the mutant allele (strategy 1a—rs10414837, strategy 1b—rs3761005) and a second guide sequence targets a sequence in intron 3 which is common to two alleles of the gene. In another, a first guide sequence targets a specific sequence of a heterozygous SNP position in an upstream region of the mutant allele (strategy 1a—rs10414837, strategy 1b—rs3761005) and a second guide sequence targets a sequence in 3′ UTR which is common to two alleles of the gene.

FIG. 3: Schematic showing excising from intron 3 or intron 4 or 3′ UTR to regions downstream to the 3′ UTR. In one example, a first guide sequence targets a specific sequence of a heterozygous SNP position in an upstream region of the mutant allele (strategy 2—rs1683564) and a second guide sequence targets a sequence in intron 4 which is common to two alleles of the gene. In another, a first guide sequence targets a specific sequence of a heterozygous SNP position in an upstream region of the mutant allele (strategy 2—rs1683564) and a second guide sequence targets a sequence in intron 3 which is common to two alleles of the gene. In a further, a first guide sequence targets a specific sequence of a heterozygous SNP position in an upstream region of the mutant allele (strategy 2—rs1683564) and a second guide sequence targets a sequence in 3′ UTR which is common to two alleles of the gene.

FIG. 4: Schematic showing excising from intron 3 or intron 4 or 3′ UTR to regions downstream to the 3′ UTR. The strategy is designed such as to specifically knock-out the disease-causing allele (‘mutant allele’), while leaving the healthy allele intact. Allele specific editing is achieved by using guides that target discriminating (heterozygous) SNP positions with relatively high heterozygosity frequency in the population.

FIG. 5: The expected coverage in the population based on heterozygotes frequency and overlap/linkage between select heterozygous SNPs. Designing alternative solutions for the three therapeutic strategies may enable a coverage of about 80% of the population. It is evaluated that 80% of the population bear one or more heterozygous SNPs from the list above. From which 33% bear one heterozygous SNP, 31% bear two heterozygous SNPs from the list, 16% bear three heterozygous SNPs and 20% do not bear any of the SNPs listed above.

FIG. 6: HeLa cells seeded into 96 well-plate (3K/well) and 24 h later were co-transfected using Turbofect reagent (Thermo Scientific) with either 65 ng of WT-Cas9 or Dead-Cas9, and 20 ng of a gRNA plasmid (identified as g36 through g66, which target different regions and SNPs in ELANE. Percent of editing was calculated according the following formula: 100%−(Intensity not edited band/Intensity total bands)*100. The mean activity of each gRNA following subtraction of the Dead-Cas9 background activity±SD of three independent experiments is shown.

FIG. 7: HSCs from healthy donors were nucleofected with RNA components of SpCas9-WT and gRNAs targeting either EMX1 (sgEMX1) or ELANE (g35: INT 4; g58: rs3761005; g62: rs1683564). 72 h post nucleofection gDNA was extracted and editing levels were assayed by IDAA. The mean % of editing±SD of two independent experiments performed in duplicates is shown.

FIG. 8: Specific knock-out of the mutated allele of the ELANE gene is mediated by excising intron 4 and exon 5 of the mutant allele of the ELANE gene. This is achieved by mediating a DSB in intron 4 and utilizing SNP rs1683564 for mediating an allele specific DSB.

FIG. 9: Intrinsic fidelity in human cells. OMNI-50 or SpCas9 nuclease were expressed in mammalian cell system by DNA transfection together with a sgRNA expressing plasmid. Cell lysates were used for site specific genomic DNA amplification and NGS. The percentage of Indels was measured and analyzed as described below for on-target vs off-target editing in HeLa cell line using ELANEg35_OMNI-50 or ELANEg62_OMNI-50 (see Table 7). In both cases the genomic On and Off target sequences are noted below the chart with PAM sequences underlined. Each experiment represents three independent repeats.

FIGS. 10A-10D: OMNI-50 activity Assay as RNP. OMNI-50 nuclease was over-expressed and purified. The purified protein was complexed with synthetic sgRNA to form RNPs. For the in-vitro assays (FIGS. 13A and 13B) RNPs were incubated with a linear DNA templates with the corresponding target and PAM sequences. Activity was verified by the ability to cleave the linear template. For the in-vivo assays (FIGS. 13C and 13D) U2OS cells were electroporated with RNP, and activity was determined as indel frequency by NGS. FIG. 10A: Activity assay of OMNI-50 RNP with different spacer lengths (17-23 nucleotides) of guide 35 (Table 4). FIG. 10B: Reducing amounts of RNPs (4, 2, 1.2, 0.6 and 0.2 μmol) with spacer lengths 20-23 nucleotides were incubated with 100 ng DNA target template. FIG. 10C: Activity assay for OMNI-50 as RNP in U2OS cells: RNPs with spacer lengths 17-23 nucleotides were electroporated into a U2OS cell line and editing levels (indels) measured by NGS. FIG. 10D: Activity assay for OMNI-50 as RNP in U2OS cells: RNPs with ELANE g35 sgRNA V1-4 were electroporated into a U2OS cell line and editing levels (indels) measured by NGS.

FIG. 11: Activity assay for OMNI-50 as RNP in iPSC: RNPs with spacer lengths 17-23 nucleotides (table 4) were electroporated into iPSC cell line and editing levels (indels) was measured by NGS.

FIG. 12: Scheme depicting excision strategy of ELANE cleaved in two locations, one in intron 4 in a non-discriminatory site (bi-allelic cut) and one in a heterozygous SNP site, rs1683564, located downstream to the 3′ UTR, which is an allele specific cut, resulting in allele specific deletion.

FIG. 13A-13D: Patient derived hematopoietic stem cells (HSC) harboring ELANE mutation G221termination (G221ter) depict excision-based rescue effect. FIG. 13A: Scheme depicting experimental workflow. Manipulated HSC electroporated with RNPs using Lonza P3 Cell Line 4D-Nucleofector™ X Kit with DZ100 program recover for 3 days in HSC rich media and subjected to differentiation by culturing for 7 days with IL-3, SCF, GMCSF & GCSF for proliferation and myeloid progenitor differentiation followed by a 7-day culture in GCSF for neutrophil differentiation. On day 14, cells were analyzed by FACS for monocytic subset represented by CD14 and neutrophilic subset represented by CD66b+CD14+ surface markers. FIG. 13B: Flow cytometry analysis of untreated and edited cells harvested from HSC-based neutrophilic differentiation from healthy and G221ter patients derived on day 14 of differentiation. Plots reveal a decrease in CD66b+CD14+ neutrophilic subset and enhanced CD14+ monocytic population in untreated patient derived differentiation in comparison to healthy volunteer. Note, excised patient HSC completely rescues neutrophil differentiation. FIG. 13C: Graph representing percent cellular composition of neutrophils & monocytes between healthy and G221ter patient donor at day 14. FIG. 13D: Graph representing excision efficiency at day 5 and 14 with sgRNA constant+sgRNA-564DS-ref measured by ddPCR from differentiated HSCs of healthy and G221ter patient cells homozygous to rs1683564, therefore, the excision is expected to be biallelic. Excision is determined by amplification of two regions in ELANE, Exon1 and Exon 5, using two different probes labeled with FAM and HEX, respectively. The ratio between the HEX and the FAM signals is translated to excision efficiency.

FIGS. 14A-14F: Patient derived hematopoietic stem cells (HSC) harboring S126L ELANE mutation depicts a rescue effect following differential editing of the mutated allele. FIG. 14A: Flow cytometry analysis of untreated and edited cells harvested from HSC-based neutrophilic differentiation from healthy and S126L patients derived on day 14 of differentiation. Plots reveal a decrease in CD66b+CD14+ neutrophilic subset and enhanced CD14+ monocytic population in untreated patient derived differentiation in comparison to healthy volunteer. Note, sgRNA constant+sgRNA-564DS-ref excised patient HSC linked to mutation completely rescues neutrophil differentiation in contrast to sgRNA constant+sgRNA-564DS-alt which depicts a partial rescue effect. FIG. 14B: Graph representing percent cellular composition of neutrophils & monocytes between healthy and S126L patient at day 14. FIG. 14C: Graph representing excision efficiency at day 4 and 14 with sgRNA constant+sgRNA-564DS-ref and sgRNA constant+sgRNA-564DS-alt measured by ddPCR from differentiated HSCs of healthy and S126L patient cells heterozygote for the rs1683564, therefore, the excision is expected to be allele specific. Excision is determined by amplification of two regions in ELANE, Exon1 and Exon 5, using two different probes labeled with FAM and HEX, respectively. The ratio between the HEX and the FAM signals is translated to excision efficiency. Note, excision efficiency with sgRNA-564DS-ref composition results in higher efficiency than sgRNA-564DS-alt. FIG. 14D: Graph representing allele specific editing at day 4 and 14 with sgRNA constant+sgRNA-564DS-ref and sgRNA constant+sgRNA-564DS-alt measured by ddPCR from differentiated HSCs of healthy and S126L patient cells heterozygote to rs1683564. Editing specificity is determined by two competitive probes, FAM probe which binds the Alternative Allele and HEX probe which binds the Reference Allele, governing decrease signal from the edited allele. Note, reduction in both reference and alternative allele when editing with sgRNA-564DS-ref indicating reduced specificity in comparison to sgRNA-564DS-alt. The ratio between the concentration of the Reference allele to the alternative allele in Heterozygote untreated cells is 1. The graph represents average concentration of reference allele (HEX), alternative allele (FAM), normalized to endogenous gene control RPP30 and STAT1 Exon 3 for each gDNA sample.

FIG. 14E: The level of ELANE's mRNA of all treatments was assessed by qRT-PCR following differentiating the cells towards neutrophils. The level of the mRNA is quantified relative to untreated cells that were not edited. FIG. 14F: IGV snapshot of allele specific transcription determined by NGS on cDNA targeting exon-4 harboring S126L mutation extracted from sgRNA constant+sgRNA-564DS-ref and sgRNA constant+sgRNA-564DS-alt excised patient HSC compared to untreated cells. Note, mutated allele specific RNA reduction correlated to sgRNA constant+sgRNA-564DS-ref excision composition was observed relative to untreated cells which show a 1:1 ratio of WT (C marked in blue)/mutation S126L (T marked in red) transcription. All samples were evaluated in triplicates.

FIG. 15: Sequences of off-target sites identified by GUIDE-seq for U2OS cells targeting ELANEg35 (sgRNA-constant) site with OMNI50 nuclease. GUIDE-seq sequencing read counts are shown to the right of each site following by target location in human hg38 reference (Chromosome number: position). The on-target site is marked with a black square.

FIG. 16: Graph showing % of Editing on constant guide panel in U2OS cells treated with SpCas9 or OMNI50 nucleases.

FIG. 17: Editing analysis of the sgRNA-564DS-ref panel in U2OS cells treated with SpCas9 or OMNI50 nucleases.

FIG. 18: Editing analysis of the constant guide off targets panel for analysis of patient treated samples with constant guide and sgRNA-564DS-ref.

FIG. 19: Editing analysis of sgRNA-564DS-ref off targets panel for analysis of patient treated samples with constant guide and sgRNA-564DS-ref.

FIG. 20: Editing analysis of sgRNA-564DS-Alt off targets panel for analysis of patient treated samples with constant guide and sgRNA-564DS-Alt.

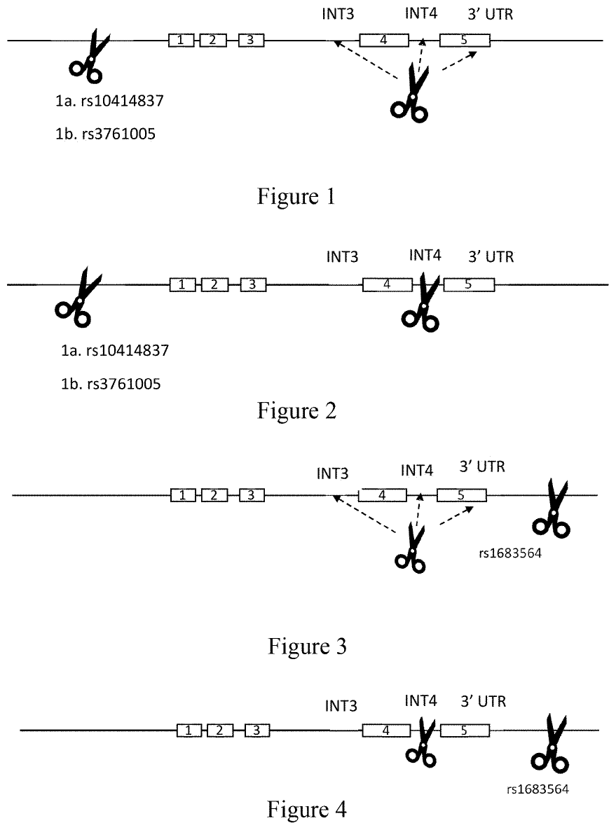

### DETAILED DESCRIPTION

Embodiments of the present invention provide a method for inactivating in a cell a mutant allele of the elastase, neutrophil expressed gene (ELANE gene) gene having a mutation associated with severe congenital neutropenia (SCN) or cyclic neutropenia (CyN) and which cell is heterozygous at one or more polymorphic sites selected from the group consisting of rs10424470, rs4807932, rs10414837, rs376107533, rs3761010, rs351108, rs3826946, rs10413889, rs3761007, rs10409474, rs3761005, rs351107, rs3761001, rs740021, rs781452480, rs371057361, rs570466264, rs1041904080, rs7250194, rs17216649, rs199720952, rs6510983, rs17223066, rs7255385, rs9749274, rs111361200, rs112639467, rs141213775, rs28591229, rs10469327, rs3834645, rs1683564, rs71335276, and rs8107095, the method comprising
introducing to the cell a composition comprising:
   a CRISPR nuclease or a sequence encoding the CRISPR nuclease; and
   a first RNA molecule comprising a guide sequence portion having 21-30 nucleotides,
wherein a complex of the CRISPR nuclease and the first RNA molecule affects a double strand break in the mutant allele of the ELANE gene.

In embodiments of the present invention the guide sequence portion of the first RNA molecule comprises 21-30 contiguous nucleotides containing nucleotides in the sequence set forth in any one of SEQ ID NOs: 1-3751.

In embodiments of the present invention wherein the cell (or subject) is heterozygous at one or more polymorphic sites selected from the group consisting of: rs9749274, rs740021, rs199720952, rs28591229, rs71335276, rs3826946, rs10413889, rs3761005, rs10409474, rs3761007, rs1683564, rs17216649, rs10469327, rs8107095, rs10414837, and rs10424470, and the guide sequence portion of the first RNA molecule comprises 21-30 contiguous nucleotides containing nucleotides in the sequence set forth in any one of SEQ ID NOs: 1-3751.

In some embodiments, the polymorphic site is rs1683564 and the guide sequence portion of the first RNA molecule comprises 21-30 contiguous nucleotides containing nucleotides in the sequence set forth in any one of SEQ ID NOs: 2517, 2601, 2464-2516, 2518-2600, 2602-2613, 1-2463, 2614-3751.

In embodiments of the present invention, the CRISPR nuclease and the first RNA molecule affects a double strand break in the mutant allele of the ELANE gene, which mutant allele is targeted for the double strand break based on the one or more polymorphic sites.

In embodiments of the present invention, the CRISPR nuclease and the first RNA molecule affects a double strand break in the mutant allele of the ELANE gene, which mutant allele is targeted for the double strand break based on a sequence of the mutant allele at the one or more polymorphic sites.

In embodiments of the present invention, the CRISPR nuclease and the first RNA molecule affects a double strand break in the mutant allele of the ELANE based on the nucleotide base of the one or more polymorphic sites present on the mutant allele of the ELANE gene.

Embodiments of the present invention further comprise introduction of a second RNA molecule comprising a guide sequence portion capable of complexing with a CRISPR nuclease, wherein the complex of the second RNA molecule and CRISPR nuclease affects a second double strand break in the ELANE gene. In an embodiment, the second RNA molecule comprises a guide sequence portion having 21-30 nucleotides.

In embodiments of the present invention, a composition may comprise 1, 2, 3 or more CRISPR nucleases or sequencing encoding the CRISPR nucleases. In embodiments of the present invention, introducing a composition to the cell may comprise introducing 1, 2, 3, or more compositions to the cell. In embodiments of the present invention, each composition may comprise a different CRISPR nuclease or sequence encoding the CRISPR nucleases or the same CRISPR nuclease or sequence encoding the CRISPR nuclease. In embodiments of the present invention involving two RNA molecules, the second RNA molecule may form a complex with the same CRISPR nuclease as the first RNA molecule, or may form a complex with another CRISPR nuclease.

In embodiments of the present invention, the second double strand break is within a non-coding region of the ELANE gene. In embodiments of the present invention, the non-coding region is in intron 4.

In embodiments of the present invention, the guide sequence portion of the first RNA molecule comprises 21 contiguous nucleotides containing nucleotides in the sequence set forth in any one of SEQ ID NOs: 1-3751.

In embodiments of the present invention, the guide sequence portion of the first RNA molecule comprises 22 contiguous nucleotides containing nucleotides in the sequence set forth in any one of SEQ ID NOs: 1-3751.

According to embodiments of the present invention, the guide sequence portion of the second RNA molecule comprises 21-30 contiguous nucleotides containing nucleotides in the sequence set forth in any one of SEQ ID NOs: 1-3751.

In embodiments of the present invention, the second double strand break is within a non-coding region of the ELANE gene.

In embodiments of the present invention, the cell is heterozygous at rs10414837 or rs3761005 and the complex of the second RNA molecule and CRISPR nuclease affects a double strand break in intron 4 of the ELANE gene.

In embodiments of the present invention, the cell is heterozygous at rs1683564 and the complex of the second RNA molecule and CRISPR nuclease affects a double strand break in intron 4 of the ELANE gene.

In some embodiments, the cell is heterozygous at the polymorphic sites in the ELANE rs10414837, and a complex of a first RNA molecule comprising a guide sequence portion having 21-30 nucleotides and a CRISPR nuclease affects a double strand break in the mutant allele of the ELANE gene and not in the functional allele of the ELANE gene. In such embodiments the guide sequence portion of the first RNA molecule may comprise a sequence of contiguous nucleotides as set forth in any one of the SEQ ID NOs targeting rs10414837 as listed in Table 1.

In some embodiments, the cell is heterozygous at the polymorphic sites in the ELANE rs3761005, and a complex of a first RNA molecule comprising a guide sequence portion having 21-22 or 21-30 nucleotides and a CRISPR nuclease affects a double strand break in the mutant allele of the ELANE gene and not in the functional allele of the ELANE gene. In such embodiments the guide sequence portion of the first RNA molecule may comprise a sequence of contiguous nucleotides as set forth in any one of the SEQ ID NOs targeting rs3761005 as listed in Table 1.

In some embodiments, the cell is heterozygous at the polymorphic sites in the ELANE rs1683564, and a complex of a first RNA molecule comprising a guide sequence portion having 21-22 or 21-30 nucleotides and a CRISPR nuclease affects a double strand break in the mutant allele of the ELANE gene and not in the functional allele of the ELANE gene. In such embodiments the guide sequence portion of the first RNA molecule may comprise a sequence of contiguous nucleotides as set forth in any one of the SEQ ID NOs targeting rs1683564 as listed in Table 1.

Embodiments of the present invention comprise obtaining the cell with an ELANE gene mutation associated with severe congenital neutropenia (SCN) or CyN from a subject with an ELANE gene mutation related to SCN or CyN and/or suffering from SCN or CyN and which subject is heterozygous at one or more polymorphic sites selected from the group consisting of: rs10424470, rs4807932, rs10414837, rs376107533, rs3761010, rs351108, rs3826946, rs10413889, rs3761007, rs10409474, rs3761005, rs351107, rs3761001, rs740021, rs781452480, rs371057361, rs570466264, rs1041904080, rs7250194, rs17216649, rs199720952, rs6510983, rs17223066, rs7255385, rs9749274, rs111361200, rs112639467, rs141213775, rs28591229, rs10469327, rs3834645, rs1683564, rs71335276, and rs8107095.

Embodiments of the present invention comprise first selecting a subject with an ELANE gene mutation related to SCN or CyN and/or suffering from SCN or CyN and which subject is heterozygous at one or more polymorphic sites selected from the group consisting of: rs10424470, rs4807932, rs10414837, rs376107533, rs3761010, rs351108, rs3826946, rs10413889, rs3761007, rs10409474, rs3761005, rs351107, rs3761001, rs740021, rs781452480, rs371057361, rs570466264, rs1041904080, rs7250194, rs17216649, rs199720952, rs6510983, rs17223066, rs7255385, rs9749274, rs111361200, rs112639467, rs141213775, rs28591229, rs10469327, rs3834645, rs1683564, rs71335276, and rs8107095, and obtaining the cell from the subject.

Embodiments of the present invention comprise obtaining the cell from the subject by mobilization and/or by apheresis.

Embodiments of the present invention comprise obtaining the cell from the subject by bone marrow aspiration.

In embodiments of the present invention, the cell is prestimulated prior to introducing the composition to the cell.

Embodiments of the present invention comprise culture expanding the cell to obtain cells.

In embodiments of the present invention, the cells are cultured with one or more of: stem cell factor (SCF), IL-3, and GM-CSF.

In embodiments of the present invention, the cells are cultured with at least one cytokine.

In embodiments of the present invention, the at least one cytokine is a recombinant human cytokine.

In embodiments of the present invention, the cell is among a plurality of cells, wherein the composition comprising the first RNA molecule or both the first and the second RNA molecule is introduced into at least the cell as well as other cells among the plurality of cells, and the mutant allele of the ELANE gene is inactivated in at least the cell as well as in the other cells among the plurality of cells, thereby obtaining multiple modified cells.

In embodiments of the present invention, introducing the composition comprising the first RNA molecule or introduction of the second RNA molecule comprises electroporation of the cell or cells.

Embodiments of the present invention provide for a modified cell obtained by the methods of the present invention.

In embodiments of the present invention, the modified cells are further culture expanded.

In embodiments of the present invention, the modified cells are capable of engraftment.

In embodiments of the invention, modified cells are capable of long-term engraftment when infused into a patient, giving rise to differentiated hematopoietic cells for at least 12 months after infusion, preferably at least 24 months and even more preferably at least 30 months after infusion. In a further embodiment, the modified cells are capable of long-term engraftment when infused into an autologous subject. In a further embodiment, the modified cells are capable of long-term engraftment when infused into a subject without myeloablation. In an embodiment of the present invention, the modified cells are delivered to a subject in sufficient numbers that, when engrafted into a human subject, provide long term engraftment.

In embodiments of the present invention, the modified cell or cells are capable of giving rise to progeny cells.

In embodiments of the present invention, the modified cell or cells are capable of giving rise to progeny cells after engraftment.

In embodiments of the present invention, the modified cell or cells are capable of giving rise to progeny cells after an autologous engraftment.

In embodiments of the present invention, the modified cell or cells are capable of giving rise to progeny cells for at least 12 months or at least 24 months after engraftment.

In one embodiment, the cell or cells are stem cells. In one embodiment, the cell is an embryonic stem cell. In some embodiment, the stem cell is a hematopoietic stem/progenitor cell (HSPC).

In embodiments of the present invention, the modified cell or cells are CD34+ hematopoietic stem cells.

In embodiments of the present invention, the modified cell or cells are bone marrow cells or peripheral mononucleated cells (PMCs).

Embodiments of the present invention provide for a modified cell lacking at least a portion of one allele of the ELANE gene.

In embodiments of the present invention, the modified cell was modified from a cell heterozygous at one or more polymorphic sites selected from the group consisting of: rs10424470, rs4807932, rs10414837, rs376107533, rs3761010, rs351108, rs3826946, rs10413889, rs3761007, rs10409474, rs3761005, rs351107, rs3761001, rs740021, rs781452480, rs371057361, rs570466264, rs1041904080, rs7250194, rs17216649, rs199720952, rs6510983, rs17223066, rs7255385, rs9749274, rs111361200, rs112639467, rs141213775, rs28591229, rs10469327, rs3834645, rs1683564, rs71335276, and rs8107095.

Embodiments of the present invention provide for a composition comprising modified cells and a pharmaceutically acceptable carrier.

Embodiments of the present invention provide for an in vitro or ex vivo method of preparing a composition, comprising mixing the cells of the present invention with the pharmaceutically acceptable carrier.

Embodiments of the present invention provide for a method of preparing in vitro or ex vivo a composition comprising modified cells, the method comprising:
a) isolating HSPCs from cells obtained from a subject with an ELANE gene mutation related to SCN or CyN and/or suffering from SCN or CyN and which subject is heterozygous at one or more polymorphic sites selected from the group consisting of: rs10424470, rs4807932, rs10414837, rs376107533, rs3761010, rs351108, rs3826946, rs10413889, rs3761007, rs10409474, rs3761005, rs351107, rs3761001, rs740021, rs781452480, rs371057361, rs570466264, rs1041904080, rs7250194, rs17216649, rs199720952, rs6510983, rs17223066, rs7255385, rs9749274, rs111361200, rs112639467, rs141213775, rs28591229, rs10469327, rs3834645, rs1683564, rs71335276, and rs8107095, and obtaining the cell from the subject;
b) introducing to the cells of step (a) a composition comprising:
   a CRISPR nuclease or a sequence encoding the CRISPR nuclease; and
   a first RNA molecule comprising a guide sequence portion having 21-30 nucleotides,
wherein a complex of the CRISPR nuclease and the first RNA molecule affects a double strand break in the mutant allele of the ELANE gene in one or more cells,
   optionally, introducing to the cells a second RNA molecule comprising a guide sequence portion capable of complexing with a CRISPR nuclease, wherein the complex of the second RNA molecule and CRISPR nuclease affects a second double strand break in the ELANE gene in the one or more cells
so as to inactive the mutant allele of the ELANE gene in one or more cells thereby obtaining modified cells; optionally
c) culture expanding the modified cells of step (b),
wherein the modified cells are capable of engraftment and giving rise to progeny cells after engraftment.

Embodiments of the present invention provide for use of a composition prepared in vitro by a method comprising:
a) isolating HSPCs from cells obtained from a subject with an ELANE gene mutation related to SCN or CyN and/or suffering from SCN or CyN and which subject is heterozygous at one or more polymorphic sites selected from the group consisting of: rs10424470, rs4807932, rs10414837, rs376107533, rs3761010, rs351108, rs3826946, rs10413889, rs3761007, rs10409474, rs3761005, rs351107, rs3761001, rs740021, rs781452480, rs371057361, rs570466264, rs1041904080, rs7250194, rs17216649, rs199720952, rs6510983, rs17223066, rs7255385, rs9749274, rs111361200, rs112639467, rs141213775, rs28591229, rs10469327, rs3834645, rs1683564, rs71335276, and rs8107095;
b) introducing to the cells of step (a) a composition comprising:
   a CRISPR nuclease or a sequence encoding the CRISPR nuclease; and
   a first RNA molecule comprising a guide sequence portion having 21-30 nucleotides,
wherein a complex of the CRISPR nuclease and the first RNA molecule affects a double strand break in the mutant allele of the ELANE gene in one or more cells,
   optionally, introducing to the cells a second RNA molecule comprising a guide sequence portion capable of complexing with a CRISPR nuclease, wherein the complex of the second RNA molecule and CRISPR nuclease affects a second double strand break in the ELANE gene in the one or more cells
so as to inactive the mutant allele of the ELANE gene in one or more cells thereby obtaining modified cells; optionally;
c) culture expanding the cells of step (b) wherein the modified cells are capable of engraftment and giving rise to progeny cells after engraftment; and
d) administering to the subject the cells of step (b) or step (c)
for treating the SCN or CyN in the subject.

Embodiments of the present invention provide for a method of treating a subject afflicted with SCN or CyN, comprising administration of a therapeutically effective amount of the modified cells, compositions, or the compositions prepared by the methods of the instant invention

Embodiments of the present invention provide for a method for treating SCN or CyN in a subject with an ELANE gene mutation relating to SCN or CYN in need thereof and which subject is heterozygous at one or more polymorphic sites selected from the group consisting of: rs10424470, rs4807932, rs10414837, rs376107533, rs3761010, rs351108, rs3826946, rs10413889, rs3761007, rs10409474, rs3761005, rs351107, rs3761001, rs740021, rs781452480, rs371057361, rs570466264, rs1041904080, rs7250194, rs17216649, rs199720952, rs6510983, rs17223066, rs7255385, rs9749274, rs111361200, rs112639467, rs141213775, rs28591229, rs10469327, rs3834645, rs1683564, rs71335276, and rs8107095, the method comprising:
a) isolating HSPCs from cells obtained from the subject;
b) introducing to the cells of step (a) a composition comprising:
   a CRISPR nuclease or a sequence encoding the CRISPR nuclease; and
   a first RNA molecule comprising a guide sequence portion having 21-30 nucleotides,
wherein a complex of the CRISPR nuclease and the first RNA molecule affects a double strand break in the mutant allele of the ELANE gene in one or more cells,
   optionally, introducing to the cells a second RNA molecule comprising a guide sequence portion capable of complexing with a CRISPR nuclease, wherein the complex of the second RNA molecule and CRISPR nuclease affects a second double strand break in the ELANE gene in the one or more cells
so as to inactive the mutant allele of the ELANE gene in one or more cells thereby obtaining modified cells; optionally;
c) culture expanding the cells of step (b) wherein the modified cells are capable of engraftment and giving rise to progeny cells after engraftment; and
d) administering to the subject the cells of step (b) or step (c)
thereby treating the SCN or CyN in the subject.

Embodiments of the present invention provide for a method for treating SCN or CyN in a subject with an ELANE gene mutation relating to SCN or CYN in need thereof and which subject is heterozygous at one or more polymorphic sites selected from the group consisting of: rs10424470, rs4807932, rs10414837, rs376107533, rs3761010, rs351108, rs3826946, rs10413889, rs3761007, rs10409474, rs3761005, rs351107, rs3761001, rs740021, rs781452480, rs371057361, rs570466264, rs1041904080, rs7250194, rs17216649, rs199720952, rs6510983, rs17223066, rs7255385, rs9749274, rs111361200, rs112639467, rs141213775, rs28591229, rs10469327, rs3834645, rs1683564, rs71335276, and rs8107095, the method comprising
administering to the subject autologous modified cells or progeny of autologous modified cells, wherein the autologous modified cells are modified so as to have a double strand break in the mutant allele of the ELANE gene,
   wherein said double strand break results from introduction to the cells of a composition comprising a CRISPR nuclease or a sequence encoding the CRISPR nuclease and a first RNA molecule wherein a complex of the CRISPR nuclease and the first RNA molecule affects a double strand break in the mutant allele of the ELANE gene so as to inactive the mutant allele of the ELANE gene in the cell,
thereby treating the SCN or CyN in the subject.

Embodiments of the present invention provide for a method of selecting a subject for treatment from a pool of subjects diagnosed with SCN or CyN, comprising the steps of:
a) obtaining cells from each subject in the pool of subjects;
b) screening each subject's cells for an ELANE gene mutation related to SCN or CyN, and selecting only subjects with an ELANE gene mutation related to SCN or CyN;
c) screening by sequencing the cells of the subjects selected in step (b) for heterozygosity at one or more polymorphic sites selected from the group consisting of: rs10414837, rs3761005, rs1683564,
d) selecting for treatment only subjects with cells heterozygous at the one of more polymorphic sites.
e) obtaining HSPC cells from the bone marrow of the subject either by aspiration or by mobilization and apheresis of peripheral blood; and
f) introducing to the HSPC cells of step (e):
   one or more CRISPR nucleases or sequences encoding the one or more CRISPR nucleases
   a first RNA molecule comprising a guide sequence portion having 21-30 contiguous nucleotides containing nucleotides in the sequence set forth in any one of SEQ ID NOs: 1-3751, targeting the nucleotide base of the heterozygous allele of the one or more polymorphic sites present on the mutant allele of the ELANE gene,
wherein a complex of the first RNA molecule and a CRISPR nuclease affects a first double strand break in the mutant allele of the ELANE gene in one or more of the HSPC cells and a complex of the second RNA molecule and a CRISPR nuclease affect a second double strand break in intron 4 of both alleles of the ELANE gene in the one or more HSPC cells in which the complex of the first RNA molecule and the CRISPR nuclease affected a first double strand break, thereby obtaining modified cells;
g) administering to the subject the modified cells of step (0,
thereby treating SCN or CyN in the subject.

Embodiments of the present invention provide an RNA molecule comprising a guide sequence portion having 21-22 or 21-30 contiguous nucleotides containing nucleotides in the sequence set forth in any one of SEQ ID NOs: 1-3751.

Embodiments of the present invention further comprise a second RNA molecule comprising a guide sequence portion.

In embodiments of the present invention, the second RNA molecule targets a non-coding region of the ELANE gene.

In embodiments of the present invention, the nucleotide sequence of the guide sequence portion of the second RNA molecule is a different nucleotide sequence from the sequence of the guide sequence portion of the first RNA molecule.

In embodiments of the present invention, the first RNA molecule further comprises a portion having a sequence which binds to a CRISPR nuclease. In embodiments of the present invention, the second RNA molecule further comprise a portion having a sequence which binds to a CRISPR nuclease.

In an embodiment of the present invention, the CRISPR nuclease has at least 100%, 99%, 98%, 97%, 96%, 95%, 94%, 93%, 92%, 91%, 90%, 85%, 80% identity to SEQ ID NO: 3789.

In embodiments of the present invention, the sequence which binds to a CRISPR nuclease is a tracrRNA sequence.

In embodiments of the present invention, the first RNA molecule further comprises a portion having a tracr mate sequence. In embodiments of the present invention, the second RNA molecule further comprises a portion having a tracr mate sequence.

In embodiments of the present invention, the first RNA molecule further comprises one or more linker portions. In embodiments of the present invention, the second RNA molecule further comprises one or more linker portions.

In embodiments of the present invention, the first RNA molecule is up to 300 nucleotides in length. In embodiments of the present invention, the second RNA molecule is up to 300 nucleotides in length.

In embodiments of the present invention, the composition further comprises one or more CRISPR nucleases or sequences encoding the one or more CRISPR nucleases. In embodiments of the present invention, the composition further comprises one or more tracrRNA molecules or sequences encoding the one or more tracrRNA molecules.

Embodiments of the present invention provide a method for inactivating in a cell a mutant ELANE allele, the method comprising delivering to the cell the RNA molecules or compositions of the present invention.

In embodiments of the present invention, the one or more CRISPR nuclease or sequences encoding the one or more CRISPR nucleases and the RNA molecule or RNA molecules are delivered to the subject and/or cells substantially at the same time or at different times.

In embodiments of the present invention, the tracrRNA molecules or sequences encoding the one or more tracrRNA molecules and the RNA molecule or RNA molecules are delivered to the subject and/or cells substantially at the same time or at different times.

In embodiments of the present invention, the method comprises removing an exon containing a disease-causing mutation from a mutant allele, wherein the first RNA molecule or the first and the second RNA molecules target regions flanking an entire exon or a portion of the exon.

In embodiments of the present invention, the method comprises removing multiple exons, the entire open reading frame of a gene, or removing the entire gene.

In embodiments of the present invention, the first RNA molecule or the first and the second RNA molecules target an alternative splicing signal sequence between an exon and an intron of a mutant allele.

In embodiments of the present invention, the second RNA molecule targets a sequence present in both a mutant allele and a functional allele.

In embodiments of the present invention, the second RNA molecule targets an intron.

In embodiments of the present invention, the method results in subjecting the mutant allele to insertion or deletion by an error prone non-homologous end joining (NHEJ) mechanism, generating a frameshift in the mutant allele's sequence.

In embodiments of the present invention, the frameshift results in inactivation or knockout of the mutant allele.

In embodiments of the present invention, the frameshift creates an early stop codon in the mutant allele or the frameshift results in nonsense-mediated mRNA decay of the transcript of the mutant allele.

In embodiments of the present invention, inactivating or treating results in a truncated protein encoded by the mutant allele and a functional protein encoded by the functional allele.

In embodiments of the present invention, the cells or the subject is heterozygous at rs10414837 or rs3761005 and wherein the complex of the second RNA molecule and CRISPR nuclease affects a double strand break in intron 4 of the ELANE gene.

In embodiments of the present invention, the cells or the subject is heterozygous at rs1683564 and wherein the complex of the second RNA molecule and CRISPR nuclease affects a double strand break in intron 4 of the ELANE gene.

Embodiments of the present invention provide use of the RNA molecules, the compositions, or the composition prepared by the method of the present invention for inactivating in a cell a mutant ELANE allele.

Embodiments of the present invention provide a medicament comprising the RNA molecules, compositions, or the compositions prepared by the methods of the instant invention for use in inactivating in a cell a mutant ELANE allele, wherein the medicament is administered by delivering to the cell the RNA molecules, compositions, or the compositions prepared by the methods of the instant invention.

Embodiments of the present invention provide for use of the methods, the modified cells, the compositions, or the compositions prepared by the methods, or the RNA molecules of the instant invention for treating ameliorating or preventing SCN or CyN in a subject having or at risk of having SCN or CyN.

Embodiments of the present invention provide for a medicament comprising the RNA molecules, compositions, compositions prepared by the methods of the instant invention, or the modified cells of the instant invention, for use in treating ameliorating or preventing SCN or CyN, wherein the medicament is administered by delivering to a subject having or at risk of having SCN or CyN the RNA molecules, compositions, compositions prepared by the methods of the instant invention, or the modified cells of the instant invention.

Embodiments of the present invention provide for a kit for inactivating a mutant ELANE allele in a cell, comprising the RNA molecules of the instant invention, a CRISPR nuclease or sequence encoding the CRISPR nuclease, and/or a tracrRNA molecule or sequence encoding the tracrRNA molecule; and instructions for delivering the RNA molecule; CRISPR nuclease or sequence encoding the CRISPR nuclease, and/or the tracrRNA or sequence encoding the tracrRNA molecule to the cell to inactivate the mutant ELANE allele in the cell.

Embodiments of the present invention provide for a kit for treating SCN or CyN in a subject, comprising the RNA molecules of the instant invention, a CRISPR nuclease or sequence encoding the CRISPR nuclease, and/or a tracrRNA molecule or sequence encoding the tracrRNA molecule; and instructions for delivering the RNA molecule; CRISPR nuclease or sequence encoding the CRISPR nuclease, and/or the tracrRNA or sequence encoding the tracrRNA molecule to a subject having or at risk of having SCN or CyN so as to treat the SCN or CyN.

Embodiments of the present invention provide for a kit for inactivating a mutant ELANE allele in a cell, comprising the compositions, the composition prepared by the methods of the instant invention, or the modified cells of the instant invention, and instructions for delivering the composition to the cell so as to inactivate the ELANE gene in the cell.

Embodiments of the present invention provide for a kit for treating SCN or CyN in a subject, comprising the composition, the compositions prepared by the methods of the instant invention, or the modified cells of the instant invention, and instructions for delivering the compositions, the compositions prepared by the methods of the instant invention, or the modified cells of the instant invention, to a subject having or at risk of having SCN or CyN so as to treat SCN or CyN.

Embodiments of the present invention provide a method or kit wherein the CRISPR nuclease in the method or kit has greater cleavage activity when used with an RNA molecule comprising a guide sequence portion having 21-23 nucleotides, compared to its cleavage activity when used with an RNA molecule comprising a guide sequence portion having 20 or fewer nucleotides, and/or 24 or more nucleotides. In embodiments, the CRISPR nuclease has greater cleavage activity when used with an RNA molecule comprising a guide sequence portion having 21-22 nucleotides, compared to its cleavage activity when used with an RNA molecule comprising a guide sequence portion having 20 or fewer nucleotides, and/or 23 or more nucleotides. In some embodiments, the CRISPR nuclease has its greatest cleavage activity when used with an RNA molecule comprising a guide sequence portion having 22 nucleotides. In some embodiments, the CRISPR nuclease has its greatest cleavage activity when used with an RNA molecule comprising a guide sequence portion having 21 nucleotides. In some embodiment, the CRISPR nuclease has its greatest cleavage activity when used with an RNA molecule comprising a guide sequence portion having 23 nucleotides. In some embodiment, the CRISPR nuclease has its greatest cleavage activity when used with an RNA molecule comprising a guide sequence portion having 24 nucleotides.

In embodiments of the present invention wherein the CRISPR nuclease has greater cleavage activity when used with an RNA molecule comprising a guide sequence portion having 21-23 nucleotides, the CRISPR nuclease has at least 95% identity to the amino acid sequence as set forth in SEQ ID NO: 3789 or the sequence encoding the CRISPR nuclease has at least a 95% sequence identity to SEQ ID NO: 3790 or SEQ ID NO: 3791. In embodiments of the present invention, the CRISPR nuclease has at least 100%, 99%, 98%, 97%, 96%, 95%, 94%, 93%, 92%, 91%, 90%, 89%, 88%, 87%, 86%, 85%, 84%, 83%, or 82% identity to the amino acid sequence as set forth in SEQ ID NO: 3789 or the sequence encoding the CRISPR nuclease has at least a 100%, 99%, 98%, 97%, 96%, 95%, 94%, 93%, 92%, 91%, 90%, 89%, 88%, 87%, 86%, 85%, 84%, 83%, or 82% sequence identity to SEQ ID NO: 3790 or SEQ ID NO: 3791.

In embodiments of the present invention, composition comprising the CRISPR nuclease has one or more of the following features: (a) the composition is in an aqueous solution, (b) the pH of the composition is between 6 and 8; and (d) the composition is free of RNAse.

### Definitions

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

Unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

As used herein, the term “heterozygous single nucleotide polymorphism” or “SNP” refers to a single nucleotide position in a genome that differs between paired chromosomes within a population. As used herein the most common or most prevalent nucleotide base at the position is referred to as the reference (REF), wild-type (WT), common, or major form. Less prevalent nucleotide bases at the position are referred to as the alternative (ALT), minor, rare, or variant forms.

The “guide sequence portion” of an RNA molecule refers to a nucleotide sequence that is capable of hybridizing to a specific target DNA sequence, e.g., the guide sequence portion has a nucleotide sequence which is fully complementary to the DNA sequence being targeted along the length of the guide sequence portion. In some embodiments, the guide sequence portion is 21-22 nucleotides in length. As described in more detail below, the guide sequence portion may also be, for example, 23, 24, 25, 26, 27, 28, 29 or 30 nucleotides in length. The entire length of the guide sequence portion is fully complementary to the DNA sequence being targeted along the length of the guide sequence portion. The guide sequence portion may be part of an RNA molecule that can form a complex with a CRISPR nuclease with the guide sequence portion serving as the DNA targeting portion of the CRISPR complex. When the DNA molecule having the guide sequence portion is present contemporaneously with the CRISPR molecule the RNA molecule is capable of targeting the CRISPR nuclease to the specific target DNA sequence. Each possibility represents a separate embodiment. An RNA molecule can be custom designed to target any desired sequence.

The term “targets” as used herein, refers to the guide sequence portion of the RNA molecule's preferential hybridization to a nucleic acid having a targeted nucleotide sequence. It is understood that the term “targets” encompasses variable hybridization efficiencies, such that there is preferential targeting of the nucleic acid having the targeted nucleotide sequence, but unintentional off-target hybridization in addition to on-target hybridization might also occur. It is understood that where an RNA molecule targets a sequence, a complex of the RNA molecule and a CRISPR nuclease molecule targets the sequence for nuclease activity.

In the context targeting a DNA sequence that is present in a plurality of cells, it is understood that the targeting encompasses hybridization of the guide sequence portion of the RNA molecule with the sequence in one or more of the cells, and also encompasses hybridization of the RNA molecule with the target sequence in fewer than all of the cells in the plurality of cells. Accordingly, it is understood that where an RNA molecule targets a sequence in a plurality of cells, a complex of the RNA molecule and a CRISPR nuclease is understood to hybridize with the target sequence in one or more of the cells, and also may hybridize with the target sequence in fewer than all of the cells. Accordingly, it is understood that the complex of the RNA molecule and the CRISPR nuclease introduces a double strand break in relation to hybridization with the target sequence in one or more cells and may also introduce a double strand break in relation to hybridization with the target sequence in fewer than all of the cells. As used herein, the term “modified cells” refers to cells in which a double strand break is effected by a complex of an RNA molecule and the CRISPR nuclease as a result of hybridization with the target sequence, i.e. on-target hybridization.

In embodiments of the present invention, RNA guide molecule may target the mutant allele based on the nucleotide base present in the polymorphic site on the mutant allele.

In embodiments of the present invention, an RNA molecule comprises a guide sequence portion having 21-22 contiguous nucleotides containing nucleotides in the sequence set forth in any one of SEQ ID NOs: 1-3751.

In embodiments of the present invention, an RNA molecule comprises a guide sequence portion having 21-25 or 21-30 contiguous nucleotides containing nucleotides in the sequence set forth in any one of SEQ ID NOs: 1-3751.

It is understood that in any of the embodiments of the present invention the guide sequence portion of an RNA molecule may comprise 21-22 contiguous nucleotides and contain any sequence set forth in any single sequence of SEQ ID NOs: 1-3751, or in any single sequence from the above groups of sequences.

As used herein, “contiguous nucleotides” set forth in a SEQ ID NO refers to nucleotides in a sequence of nucleotides in the order set forth in the SEQ ID NO without any intervening nucleotides.

In embodiments of the present invention, the guide sequence portion may be 21 or 22 nucleotides in length and consists of 21 or 22 nucleotides in the sequences set forth in any one of SEQ ID NOs: 1-3751. In embodiments of the present invention, the guide sequence portion may be less than 21 nucleotides in length. For example, in embodiments of the present invention the guide sequence portion may be 17, 18, 19 or 20 nucleotides in length. In such embodiments the guide sequence portion may consist of 17, 18, 19 or 20 nucleotides, respectively, in the sequences set forth in any one of SEQ ID NOs: 1-3751.

In embodiments of the present invention, the guide sequence portion may be 21 nucleotides in length and contains contiguous nucleotides within the sequence set forth in any one of SEQ ID NOs: 1-3751. In embodiments of the present invention, the guide sequence portion may be 22 nucleotides in length and contains contiguous nucleotides within the sequence set forth in any one of SEQ ID NOs: 1-3751. In embodiments of the present invention, the guide sequence portion may be 21-30 nucleotides in length and contains contiguous nucleotides within the sequence set forth in any one of SEQ ID NOs: 1-3751.

In embodiments of the present invention, the guide sequence portion may be greater than 22 nucleotides in length. For example, in embodiments of the present invention the guide sequence portion may be 23, 24, 25, 26, 27, 28, 29 or 30 nucleotides in length. In such embodiments the guide sequence portion comprises 21-22 contiguous nucleotides containing nucleotides in the sequence set forth in any one of SEQ ID NOs: 1-3751, and additional nucleotides fully complimentary to a nucleotide or sequence of nucleotides adjacent to the 3′ end of the target sequence, 5′ end of the target sequence, or both.

In embodiments of the present invention, a CRISPR nuclease and an RNA molecule comprising a guide sequence portion form a CRISPR complex that binds to a target DNA sequence to effect cleavage of the target DNA sequence. CRISPR nucleases, e.g. Cpf1, may form a CRISPR complex comprising a CRISPR nuclease and RNA molecule without a further tracrRNA molecule. Alternatively, CRISPR nucleases, e.g. Cas9, may form a CRISPR complex between the CRISPR nuclease, an RNA molecule, and a tracrRNA molecule.

In embodiments of the present invention, the RNA molecule may further comprise the sequence of a tracrRNA molecule. Such embodiments may be designed as a synthetic fusion of the guide portion of the RNA molecule and the trans-activating crRNA (tracrRNA). (See Jinek (2012) Science). Embodiments of the present invention may also form CRISPR complexes utilizing a separate tracrRNA molecule and a separate RNA molecule comprising a guide sequence portion.

In such embodiments the tracrRNA molecule may hybridize with the RNA molecule via base pairing and may be advantageous in certain applications of the invention described herein.

The term “tracr mate sequence” refers to a sequence sufficiently complementary to a tracrRNA molecule so as to hybridize to the tracrRNA via basepairing and promote the formation of a CRISPR complex. (See U.S. Pat. No. 8,906,616). In embodiments of the present invention, the RNA molecule may further comprise a portion having a tracr mate sequence.

According to embodiments of the present invention, an RNA molecule may be up to 300, 290, 280, 270, 260, 250, 240, 230, 220, 210, 200, 190, 180, 170, 160, 150, 140, 130, 120, 110, or 100 nucleotides in length. Each possibility represents a separate embodiment. In embodiments of the present invention, the RNA molecule may be 17 up to 300 nucleotides in length, 100 up to 300 nucleotides in length, 150 up to 300 nucleotides in length, 200 up to 300 nucleotides in length, 100 to 200 nucleotides in length, or 150 up to 250 nucleotides in length. Each possibility represents a separate embodiment.

A “gene,” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Eukaryotic” cells include, but are not limited to, fungal cells (such as yeast), plant cells, animal cells, mammalian cells and human cells.

As used herein, the term HSPC refers to both hematopoietic stem cells and hematopoietic stem progenitor cells. Non-limiting examples of stem cells include a bone marrow cell, a myeloid progenitor cell, a multipotent progenitor cell, a lineage restricted progenitor cell.

As used herein, “progenitor cell” refers to a lineage cell that is derived from stem cell and retains mitotic capacity and multipotency (e.g., can differentiate or develop into more than one but not all types of mature lineage of cell). As used herein “hematopoiesis” or “hemopoiesis” refers to the formation and development of various types of blood cells (e.g., red blood cells, megakaryocytes, myeloid cells (e.g., monocytes, macrophages and neutrophil), and lymphocytes) and other formed elements in the body (e.g., in the bone marrow).

The term “nuclease” as used herein refers to an enzyme capable of cleaving the phosphodiester bonds between the nucleotide subunits of nucleic acid. A nuclease may be isolated or derived from a natural source. The natural source may be any living organism. Alternatively, a nuclease may be a modified or a synthetic protein which retains the phosphodiester bond cleaving activity. Gene modification can be achieved using a nuclease, for example a CRISPR nuclease.

In embodiments of the present invention, an RNA molecule is designed to target a heterozygous polymorphic site present in the mutant allele of the ELANE gene, wherein the RNA molecule targets the nucleotide base, REF or ALT, of the heterozygous polymorphic site present in the mutant allele of the ELANE gene

The present disclosure provides a method for utilizing at least one naturally occurring nucleotide difference or polymorphism (e.g., single nucleotide polymorphism (SNP)) for distinguishing/discriminating between two alleles of a gene, one allele bearing a mutation such that it encodes a mutated protein causing a disease phenotype (“mutant allele”), and the other allele encoding for a functional protein (“functional allele”). The method further comprises the step of knocking out expression of the mutated protein and allowing expression of the functional protein. In some embodiments, the method is for treating, ameliorating, or preventing a dominant negative genetic disorder.

Embodiments of the present invention provide methods for utilizing at least one heterozygous SNP in a gene expressing a dominant mutant allele in a given cell or subject. In embodiments of the present invention, the SNP utilized may or may not be associated with a disease phenotype. In embodiments of the present invention, an RNA molecule comprising a guide sequence targets the mutant allele of the gene by targeting the nucleotide base present at a heterozygous SNP in the mutant allele of the gene and therefore having a different nucleotide base in the functional allele of the gene.

According to embodiments of the present invention, the first RNA molecule targets a first heterozygous SNP present in an exon or promoter of the ELANE gene wherein the first RNA molecule targets the nucleotide base, REF or ALT, of the first SNP present in the mutant allele of the ELANE gene, and wherein the second RNA molecule targets a second heterozygous SNP present in the same or a different exon or an intron of the ELANE gene wherein the second RNA molecule targets the nucleotide base, REF or ALT, of the second SNP present in the mutant allele of the ELANE gene, or a the second RNA molecule targets a sequence in a non-coding region present in both the mutant or functional allele.

According to embodiments of the present invention, the first RNA molecule or the first and the second RNA molecules target a heterozygous SNP present in the promoter region, the start codon, or the untranslated region (UTR) of the ELANE gene wherein the RNA molecule targets the nucleotide base, REF or ALT, of the SNP present in the mutant allele of the ELANE gene.

According to embodiments of the present invention, the first RNA molecule or the first and the second RNA molecules targets at least a portion of the promoter and/or the start codon and/or a portion of the UTR of the mutant allele of the ELANE gene.

According to embodiments of the present invention, the first RNA molecule targets a portion of the promoter, a first heterozygous SNP present in the promoter of the ELANE gene, or a heterozygous SNP present upstream to the promoter of the ELANE gene and the second RNA molecule targets a second heterozygous SNP, which is present in the ELANE gene downstream of the first heterozygous SNP, and is in the promoter, in the UTR, or in an intron or in an exon of the ELANE gene, wherein the first RNA molecule targets the nucleotide base, REF or ALT, of the first SNP present in the mutant allele of the of the ELANE gene, wherein the second RNA molecule targets the nucleotide base, REF or ALT, of the second SNP present in the mutant allele of the ELANE gene.

According to embodiments of the present invention, the first RNA molecule targets a heterozygous SNP present in the promoter, upstream of the promoter, or the UTR of a the ELANE gene wherein the RNA molecule targets the nucleotide base, REF or ALT, of the SNP present in the mutant allele of the ELANE gene and the second RNA molecule is designed to target a sequence which is present in an intron of both the mutant allele and the functional allele of the ELANE gene.

According to embodiments of the present invention, the first RNA molecule targets a sequence upstream of the promotor which is present in both a mutant and functional allele of the ELANE gene and the second RNA molecule targets a heterozygous SNP present in any location of the of the ELANE gene wherein the second RNA molecule targets the nucleotide base, REF or ALT, of the SNP present in the mutant allele of the ELANE gene.

According to embodiments of the present invention, there is provided a method comprising removing an exon containing a disease-causing mutation from a mutant allele, wherein the first RNA molecule or the first and the second RNA molecules target regions flanking an entire exon or a portion of the exon.

According to embodiments of the present invention, there is provided a method comprising removing multiple exons, the entire open reading frame of a gene, or removing the entire gene.

According to embodiments of the present invention, the first RNA molecule targets a first heterozygous SNP present in an exon or promoter of the ELANE gene, and wherein the second RNA molecule targets a second heterozygous SNP present in the same or a different exon or in an intron of the ELANE gene wherein the second RNA molecule targets the nucleotide base, REF or ALT, of the second SNP present in the mutant allele of the ELANE gene, or the second RNA molecule targets a sequence in an intron present in both the mutant and functional allele of the ELANE gene.

According to embodiments of the present invention, the first RNA molecule or the first and the second RNA molecules target an alternative splicing signal sequence between an exon and an intron of a mutant allele.

According to embodiments of the present invention, the second RNA molecule targets a sequence present in both a mutant allele and a functional allele of the ELANE gene.

According to embodiments of the present invention, the second RNA molecule targets an intron.

According to embodiments of the present invention, there is provided a method comprising subjecting the mutant allele to insertion or deletion by an error prone non-homologous end joining (NHEJ) mechanism, generating a frameshift in the mutant allele's sequence.

According to embodiments of the present invention, the frameshift results in inactivation or knockout of the mutant allele.

According to embodiments of the present invention, the frameshift creates an early stop codon in the mutant allele.

According to embodiments of the present invention, the frameshift results in nonsense-mediated mRNA decay of the transcript of the mutant allele.

According to embodiments of the present invention, the inactivating or treating results in a truncated protein encoded by the mutant allele and a functional protein encoded by the functional allele.

The compositions and methods of the present disclosure may be utilized for treating, preventing, ameliorating, or slowing progression of SCN or CyN.

In some embodiments, a mutant allele is deactivated by delivering to a cell an RNA molecule which targets a heterozygous SNP present in the promoter region, the start codon, or the untranslated region (UTR) of the ELANE gene wherein the RNA molecule targets the nucleotide base, REF or ALT, of the SNP present in the mutant allele of the ELANE gene.

In some embodiments, a mutant allele is inactivated by removing at least a portion of the promoter and/or removing the start codon and/or a portion of the UTR. In some embodiments, the method of deactivating a mutant allele comprises removing at least a portion of the promoter. In such embodiments one RNA molecule may be designed for targeting a first heterozygous SNP present in the promoter or upstream to the promoter of the ELANE gene and another RNA molecule is designed to target a second heterozygous SNP, which is downstream of the first SNP, and is present in the promoter, in the UTR, or in an intron or in an exon of the ELANE gene. Alternatively, one RNA molecule may be designed for targeting a heterozygous SNP present in the promoter, or upstream of the promoter, or the UTR of the ELANE gene and another RNA molecule is designed to target a sequence which is present in an intron of both the mutant allele and the functional allele of the ELANE gene. Alternatively, one RNA molecule may be designed for targeting a sequence upstream of the promotor which is present in both the mutant and functional allele and the other guide is designed to target a heterozygous SNP present in any location of the ELANE gene e.g., in an exon, intron, UTR, or downstream of the promoter of the ELANE gene wherein the RNA molecule targets the nucleotide base, REF or ALT, of the SNP present in the mutant allele of the ELANE gene.

In some embodiments, the method of deactivating a mutant allele comprises an exon skipping step comprising removing an exon containing a disease-causing mutation from the mutant allele. Removing an exon containing a disease-causing mutation in the mutant allele requires two RNA molecules which target regions flanking the entire exon or a portion of the exon. Removal of an exon containing the disease-causing mutation may be designed to eliminate the disease-causing action of the protein while allowing for expression of the remaining protein product which retains some or all of the wild-type activity. As an alternative to single exon skipping, multiple exons, the entire open reading frame or the entire gene can be excised using two RNA molecules flanking the region desired to be excised.

In some embodiments, the method of deactivating a mutant allele comprises delivering two RNA molecules to a cell, wherein one RNA molecule targets a first heterozygous SNP present in an exon or promoter of the ELANE gene wherein the RNA molecule targets the nucleotide base, REF or ALT, of the first SNP present in the mutant allele of the ELANE gene, and wherein the other RNA molecule targets a second heterozygous SNP present in the same or a different exon or in an intron of the ELANE gene wherein the RNA molecule targets the nucleotide base, REF or ALT, of the second SNP present in the mutant allele of the ELANE gene, or the second RNA molecule targets a sequence in an intron present in both the mutant or functional allele.

In some embodiments, an RNA molecule is used to target a CRISPR nuclease to an alternative splicing signal sequence between an exon and an intron of a mutant allele, thereby destroying the alternative splicing signal sequence in the mutant allele.

Any one of, or combination of, the above-mentioned strategies for deactivating a mutant allele may be used in the context of the invention.

Additional strategies may be used to deactivate a mutant allele. For example, in embodiments of the present invention, an RNA molecule is used to direct a CRISPR nuclease to an exon or a splice site of a mutant allele in order to create a double-stranded break (DSB), leading to insertion or deletion of nucleotides by an error-prone non-homologous end-joining (NHEJ) mechanism and formation of a frameshift mutation in the mutant allele. The frameshift mutation may result in: (1) inactivation or knockout of the mutant allele by generation of an early stop codon in the mutant allele, resulting in generation of a truncated protein; or (2) nonsense mediated mRNA decay of the transcript of the mutant allele. In further embodiments, one RNA molecule is used to direct a CRISPR nuclease to a promotor of a mutant allele.

In some embodiments, the method of deactivating a mutant allele further comprises enhancing activity of the functional protein such as by providing a protein/peptide, a nucleic acid encoding a protein/peptide, or a small molecule such as a chemical compound, capable of activating/enhancing activity of the functional protein.

According to some embodiments, the present disclosure provides an RNA molecule which binds to/associates with and/or directs the RNA guided DNA nuclease e.g., CRISPR nuclease to a sequence comprising at least one nucleotide which differs between a mutant allele and a functional allele (e.g., heterozygous SNP) of a gene of interest (i.e., a sequence of the mutant allele which is not present in the functional allele).

In some embodiments, the method comprises the steps of: contacting a mutant allele of a gene of interest with an allele-specific RNA molecule and a CRISPR nuclease e.g., a Cas9 protein, wherein the allele-specific RNA molecule and the CRISPR nuclease e.g., Cas9 associate with a nucleotide sequence of the mutant allele of the gene of interest which differs by at least one nucleotide from a nucleotide sequence of a functional allele of the gene of interest, thereby modifying or knocking-out the mutant allele.

In some embodiments, the allele-specific RNA molecule and a CRISPR nuclease is introduced to a cell encoding the gene of interest. In some embodiments, the cell encoding the gene of interest is in a mammalian subject. In some embodiments, the cell encoding the gene of interest is in a plant.

In some embodiments, the cleaved mutant allele is further subjected to insertion or deletion (indel) by an error prone non-homologous end joining (NHEJ) mechanism, generating a frameshift in the mutant allele's sequence. In some embodiments, the generated frameshift results in inactivation or knockout of the mutant allele. In some embodiments, the generated frameshift creates an early stop codon in the mutant allele and results in generation of a truncated protein. In such embodiments, the method results in the generation of a truncated protein encoded by the mutant allele and a functional protein encoded by the functional allele. In some embodiments, a frameshift generated in a mutant allele using the methods of the invention results in nonsense-mediated mRNA decay of the transcript of the mutant allele.

In some embodiments, the mutant allele is an allele of the “elastase, neutrophil expressed” gene (ELANE gene). In some embodiments, the RNA molecule targets a heterozygous SNP of the ELANE gene which co-exists with/is genetically linked to the mutated sequence associated with SCN or CyN genetic disorder. In some embodiments, the RNA molecule targets a heterozygous SNP of the ELANE gene, wherein the heterozygosity of said SNP is highly prevalent in the population. In embodiments of the present invention, the REF nucleotide is prevalent in the mutant allele and not in the functional allele of an individual subject to be treated. In embodiments of the present invention, the ALT nucleotide is prevalent in the mutant allele and not in the functional allele of an individual subject to be treated. In some embodiments, a disease-causing mutation within a mutant ELANE allele is targeted.

In embodiments of the present invention, the heterozygous SNP may or may not be associated with an ELANE related disease phenotype. In embodiments of the present invention, the heterozygous SNP is associated with an ELANE related disease phenotype. In embodiments of the present invention, the SNP is not associated with an ELANE related disease phenotype

In some embodiments, the heterozygous SNP is within an exon of the gene of interest. In such embodiments, a guide sequence portion of an RNA molecule may be designed to target the exon of the gene of interest.

In some embodiments, a heterozygous SNP is within an intron or an exon of the gene of interest. In some embodiments, a heterozygous SNP is in a splice site between the intron and the exon. In some embodiments a heterozygous SNP is in a PAM site of the gene of interest.

A skilled artisan will appreciate that in each of the embodiments of the present invention, individually, each of the RNA molecules of the present invention are capable of complexing with a nuclease, e.g. a CRISPR nuclease, such as to associate with a target genomic DNA sequence of interest next to a protospacer adjacent motif (PAM). The nuclease then mediates cleavage of target DNA to create a double-stranded break within the protospacer. Accordingly, in embodiments of the present invention, the guide sequences and RNA molecules of the present invention may target a location 1, 2, 3, 4, 5, 6, 7, 8, 9, 10, 11, 12, 13, 14, 15, 16, 17, 18, 19, 20, 21, 22, 23, 24, 25, 26, 27, or 28 nucleotides upstream or downstream from a PAM site.

Therefore, in embodiments of the present invention, the RNA molecules of the present invention in complex with a nuclease, e.g., a CRISPR nuclease, may affect a double strand break in an allele of a gene 1, 2, 3, 4, 5, 6, 7, 8, 9, 10, 11, 12, 13, 14, 15, 16, 17, 18, 19, 20, 21, 22, 23, 24, 25, 26, 27, 28 upstream or downstream from a target site. A skilled artisan will appreciate that where a heterozygous polymorphic site is present and is used to define the target, an RNA molecule may be designed to target and affect a double stranded break in only the REF or ALT nucleotide base of the heterozygous polymorphic site.

Where the heterozygous polymorphic site is within the PAM site, it is understood that the RNA molecule may be designed to target a sequence 1, 2, 3, 4, 5, 6, 7, 8, 9, 10, 11, 12, 13, 14, 15, 16, 17, 18, 19, 20, 21, 22, 23, 24, 25, 26, 27, or 28 nucleotides upstream or downstream from the PAM site, and a complex of the RNA molecule and nuclease is designed to target only one of the REF or ALT nucleotide base of the heterozygous polymorphic site in the PAM site and effect a break in the PAM site, e.g. the tracrRNA is designed to target one of the REF or ALT nucleotide base of the heterozygous polymorphic site.

In embodiments of the present invention, an RNA molecule is designed to target a heterozygous polymorphic site present in the ELANE gene, wherein the RNA molecule and/or the complex of the RNA molecule and a CRISPR nuclease targets the nucleotide base, REF or ALT, of the heterozygous polymorphic site present in the mutant allele of the ELANE gene

In embodiments of the present invention, the RNA molecules, compositions, methods, cells, kits, or medicaments are utilized for treating a subject having a disease phenotype resulting from the heterozygote ELANE gene. In embodiments of the present invention, the disease is SCN or CyN. In such embodiments, the method results in improvement, amelioration or prevention of the disease phenotype.

In embodiments of the present invention, the RNA molecules, compositions, methods, cells, kits, or medicaments of the present invention are utilized in combination with a second therapy for SCN or CyN to treat the subject. In embodiments of the present invention, the RNA molecules, compositions, methods, kits, or medicaments of the present invention are administered prior to administration of the second therapy, during administration of the second therapy, and/or after administration of the second therapy.

In embodiments of the present invention, the RNA molecules, compositions, methods, cells, kits, or medicaments of the present invention are administered in combination with Granulocyte colony-stimulating factor (G-CSF) therapy.

Embodiments referred to above refer to a CRISPR nuclease, RNA molecule(s), and tracrRNA being effective in a subject or cells at the same time. The CRISPR, RNA molecule(s), and tracrRNA can be delivered substantially at the same time or can be delivered at different times but have effect at the same time. For example, this includes delivering the CRISPR nuclease to the subject or cells before the RNA molecule and/or tracr RNA is substantially extant in the subject or cells.

According to embodiments of the present invention, there is provided a method for inactivating in a cell a mutant allele of the ELANE gene, the method comprising the steps of:
a) selecting a cell with an ELANE gene mutation associated with SCN or CyN and who is heterozygous at one or more polymorphic sites in the ELANE gene selected from the group consisting of: rs10424470, rs4807932, rs10414837, rs376107533, rs3761010, rs351108, rs3826946, rs10413889, rs3761007, rs10409474, rs3761005, rs351107, rs3761001, rs740021, rs781452480, rs371057361, rs570466264, rs1041904080, rs7250194, rs17216649, rs199720952, rs6510983, rs17223066, rs7255385, rs9749274, rs111361200, rs112639467, rs141213775, rs28591229, rs10469327, rs3834645, rs1683564, rs71335276, and rs8107095;
b) introducing to the cell a composition comprising:
   a CRISPR nuclease or sequence encoding the CRISPR nuclease, and
   a first RNA molecule comprising a guide sequence portion having 21-30 nucleotides,
wherein a complex of the CRISPR nuclease and the first RNA molecule affects a double strand break in the mutant allele of the ELANE gene and not in the functional allele of the ELANE gene in the cell;
thereby inactivating the mutant allele of the ELANE gene in the cell.

According to embodiments of the present invention, there is provided a method for inactivating in a cell a mutant allele of the ELANE gene, the method comprising the steps of:
a) selecting a cell with an ELANE gene mutation associated with SCN or CyN and who is heterozygous at one or more polymorphic sites in the ELANE gene selected from the group consisting of: rs10424470, rs4807932, rs10414837, rs376107533, rs3761010, rs351108, rs3826946, rs10413889, rs3761007, rs10409474, rs3761005, rs351107, rs3761001, rs740021, rs781452480, rs371057361, rs570466264, rs1041904080, rs7250194, rs17216649, rs199720952, rs6510983, rs17223066, rs7255385, rs9749274, rs111361200, rs112639467, rs141213775, rs28591229, rs10469327, rs3834645, rs1683564, rs71335276, and rs8107095;
b) introducing to the cell a composition comprising:
   a CRISPR nuclease or sequence encoding the CRISPR nuclease, and
   a first RNA molecule comprising a guide sequence portion having 21-30 nucleotides,
wherein a complex of the CRISPR nuclease and the first RNA molecule affects a double strand break in the mutant allele of the ELANE gene and not in the functional allele of the ELANE gene in the cell;
and wherein the method further comprises introduction of a second RNA molecule comprising a guide sequence portion capable of complexing with a CRISPR nuclease, wherein the complex of the second RNA molecule and the CRISPR nuclease affects a second double strand break in the ELANE gene;
   thereby inactivating the mutant allele of the ELANE gene in the cell.

According to embodiments of the present invention, there is provided a method for inactivating in a cell a mutant allele of the ELANE gene having a mutation associated with SCN or CyN and which cell is heterozygous at one or more polymorphic sites in the ELANE gene selected from the group consisting of: rs10424470, rs4807932, rs10414837, rs376107533, rs3761010, rs351108, rs3826946, rs10413889, rs3761007, rs10409474, rs3761005, rs351107, rs3761001, rs740021, rs781452480, rs371057361, rs570466264, rs1041904080, rs7250194, rs17216649, rs199720952, rs6510983, rs17223066, rs7255385, rs9749274, rs111361200, rs112639467, rs141213775, rs28591229, rs10469327, rs3834645, rs1683564, rs71335276, and rs8107095, the method comprising
introducing to the cell a composition comprising:
   a CRISPR nuclease or sequence encoding the CRISPR nuclease, and
   a first RNA molecule comprising a guide sequence portion having 21-30 nucleotides,
wherein a complex of the CRISPR nuclease and the first RNA molecule affects a double strand break in the mutant allele of the ELANE gene;
thereby inactivating the mutant allele of the ELANE gene in the cell.

According to embodiments of the present invention, there is provided a method for inactivating in a cell a mutant allele of the ELANE gene with an ELANE gene mutation associated with SCN or CyN and heterozygous at one or more polymorphic sites in the ELANE gene selected from the group consisting of: rs10424470, rs4807932, rs10414837, rs376107533, rs3761010, rs351108, rs3826946, rs10413889, rs3761007, rs10409474, rs3761005, rs351107, rs3761001, rs740021, rs781452480, rs371057361, rs570466264, rs1041904080, rs7250194, rs17216649, rs199720952, rs6510983, rs17223066, rs7255385, rs9749274, rs111361200, rs112639467, rs141213775, rs28591229, rs10469327, rs3834645, rs1683564, rs71335276, and rs8107095, the method comprising:
introducing to the cell a composition comprising:
   a CRISPR nuclease or sequence encoding the CRISPR nuclease, and
   a first RNA molecule comprising a guide sequence portion having 21-30 nucleotides,
wherein a complex of the CRISPR nuclease and the first RNA molecule affects a double strand break in the mutant allele of the ELANE gene;
and wherein the method further comprises introduction of a second RNA molecule comprising a guide sequence portion capable of complexing with a CRISPR nuclease, wherein the complex of the second RNA molecule and CRISPR nuclease affects a second double strand break in the ELANE gene;
thereby inactivating the mutant allele of the ELANE gene in the cell.

In embodiments of the present invention, a complex of the CRISPR nuclease and the first RNA molecule affects a double strand break in the mutant allele of the ELANE gene and not in the functional allele of the ELANE gene in the cell.

In embodiments of the present invention, the cell is also heterozygous at least one additional polymorphic site in the ELANE gene selected from the group consisting of rs10424470, rs4807932, rs10414837, rs376107533, rs3761010, rs351108, rs3826946, rs10413889, rs3761007, rs10409474, rs3761005, rs351107, rs3761001, rs740021, rs781452480, rs371057361, rs570466264, rs1041904080, rs7250194, rs17216649, rs199720952, rs6510983, rs17223066, rs7255385, rs9749274, rs111361200, rs112639467, rs141213775, rs28591229, rs10469327, rs3834645, rs1683564, rs71335276, and rs8107095.

In embodiments of the present invention, a cell with an ELANE gene mutation associated with SCN or CyN may be from a subject with the ELANE gene mutation and/or afflicted with SCN or CyN. Accordingly, selecting a cell with an ELANE gene mutation may comprise selecting a subject with the ELANE gene mutation. In further embodiments of the present invention, selecting a cell may comprise selecting a cell from a subject with the ELANE gene mutation. In embodiments of the present invention, introducing the compositions of the subject invention to the cell may comprise introducing the compositions of the invention to the cell of a subject afflicted with the ELANE gene mutation.

Accordingly, in embodiments of the present invention, there is provided a method for inactivating in a cell a mutant allele of the ELANE gene of a subject, the method comprising the step of selecting a subject with an ELANE gene mutation resulting in SCN or CyN and who is heterozygous at one or more polymorphic sites in the ELANE gene selected from the group consisting of: rs10424470, rs4807932, rs10414837, rs376107533, rs3761010, rs351108, rs3826946, rs10413889, rs3761007, rs10409474, rs3761005, rs351107, rs3761001, rs740021, rs781452480, rs371057361, rs570466264, rs1041904080, rs7250194, rs17216649, rs199720952, rs6510983, rs17223066, rs7255385, rs9749274, rs111361200, rs112639467, rs141213775, rs28591229, rs10469327, rs3834645, rs1683564, rs71335276, and rs8107095.

Accordingly, embodiments of the present invention encompass the screening of subjects or cells for the ELANE gene. A person having ordinary skill in the art would readily understand methods of screening for mutations within the ELANE gene in the art, by way of non-limiting examples, e.g., sequencing-by-synthesis, Sanger sequencing, karyotyping, Fluorescence In situ Hybridization, and/or microarray testing. In embodiments of the present invention, mutations within the ELANE gene are screened by exon sequencing.

In embodiments of the present invention, the subject is or has been diagnosed with SCN or CyN by measuring the absolute neutrophil count (ANC) in peripheral blood. In embodiments of the present invention, SCN is or was diagnosed before the subject reaches the age 6 months. In embodiments of the present invention, CyN is or was diagnosed between the ages of 12 and 24 months, or after the age of 24 months. In embodiments of the present invention, SCN or Cyn is diagnosed by one or more of recurrent acute stomatologic disorders. In embodiments of the present invention, SCN or CyN is diagnosed by bone marrow examination, preferably the bone marrow examination is a cytogenetic bone marrow study. In embodiments of the present invention, SCN or CyN is diagnosed by one or more of: antineutrophil antibody assay, immunoglobulin assay (Ig GAM), lymphocyte immunophenotyping, pancreatic markers (serum trypsinogen and fecal elastase) and liposoluble vitamin levels (vitamins A, E and D). It is understood that any diagnostic method may be used with any other diagnostic method.

In embodiments of the present invention, a subject diagnosed with SCN or CyN is screened by Exon sequencing to identify an ELANE pathogenic mutation in the ELANE gene. In further embodiments the subject is screened by Sanger sequencing to confirm heterozygocity of at least one SNP in Table 1. In embodiments of the present invention, the SNP is one of rs1683564, rs10414837, and rs3761005. In embodiments of the present invention, the nucleotide of the heterozygous SNP on the mutant allele of the ELANE gene determined using BAC bio. In embodiments of the present invention, appropriate guides are selected according to Table 1. In embodiments of the present invention, the guides selected are introduced to cells, e.g. PBMCs, obtained from the subject and reduction in the pathogenic ELANE mutation in the cells is measured by, e.g. Next Generation Sequencing.

It is understood that the CRISPR/Cas9 gene editing system enables targeting the nuclease to a target site in a sequence specific manner to address disease-causing mutations. Hematopoietic stem and progenitor cells (HSPCs) have therapeutic potential because of their ability to both self-renew and differentiate (Yu, Natanson, and Dunbar 2016). Accordingly, embodiments of the present invention apply genome editing to HSPCs.

In embodiments of the present invention, an autologous therapy and utilizes autologous CD34+ hematopoietic stem cells from patients diagnosed with SCN or CyN which are edited with CRISPR/Cas9. In embodiments of the present invention, CD34+ cells are isolated from bone marrow or peripheral blood mononucleated cells (PBMCs) following patient apheresis.

In the case of dominant negative (or compound heterozygous) indications, such as SCN or CyN, the strategy is to edit the mutant allele and avoid cleavage in the non-mutant allele or other off targets by targeting a heterozygous SNP sequence.

Embodiments of the present invention may include the following steps:
selection of a patient diagnosed with SCN or CyN identified as exhibiting heterozygosity in at least one of the SNPs of Table 1 hereinbelow. In embodiments of the present invention, the subject is heterozygous at rs10414837, rs3761005, or rs1683564;
selection of a therapeutic strategy based on the identified heterozygous SNP position of the candidate patient;
obtaining HSPC cells from the bone marrow of the subject either by aspiration or by mobilization and apheresis of peripheral blood, optionally, the HSPC cells are processed (e.g., enriched, stimulated, both);
introducing into the HSPC cells (e.g., by ex vivo electroporation) a composition comprising:
   a CRISPR nuclease or a sequence encoding the same (e.g., mRNA),
   a discriminatory RNA molecule that targets a particular sequence in the identified heterozygous SNP position of the mutant allele (REF/ALT sequence), and
   a non-discriminatory RNA molecule targeting a sequence in intron 4, which is common to both the mutant allele and the other allele,
   thereby editing the HSPC cells to knockout expression of mutant ELANE allele; and
introducing the edited HSPC to the candidate patient.

In embodiments of the present invention, CD34+ cells may be isolated from bone marrow or peripheral blood mononucleated cells (PBMCs) following patient apheresis. Bone marrow or PBMCs may be collected from the patient by apheresis following HSPC mobilization. In embodiments of the invention the apheresis product may be washed to remove platelets and a CD34+ cell population may be enriched via purification using, e.g. a CliniMACS system (Miltenyi Biotec). In embodiments of the present invention, the selected cells may be prestimulated ex vivo, e.g. with a mixture of recombinant human cytokines. In embodiments of the present invention, the cells may undergo electroporation. In embodiments of the present invention, prior to electroporation, stimulated cells (e.g. CD34+ cells), the CRISPR nuclease mRNA and gRNA may be preincubated under defined conditions. In embodiments of the present invention, the cells are electroporated ex vivo with the CRISPR nuclease mRNA/gRNA mixture or with a preassembled RNPs (Ribonuclease protein of the CRISPR nuclease protein and gRNA), followed by cell washing. In embodiments of the present invention, the cells are suspended into a final formulation. In embodiments of the present invention, the cells may be resuspended. In embodiments of the present invention, the resuspended cells may be filled into bags for infusion. In embodiments of the present invention, the bags may be frozen using a freeze down step in a controlled rate freezer and/or stored in the vapor phase of liquid nitrogen. In embodiments of the present invention, the product may be administered by intravenous (IV) administration to a patient.

### Dominant Genetic Disorders

One of skill in the art will appreciate that all subjects with any type of heterozygote genetic disorder (e.g., dominant genetic disorder) may be subjected to the methods described herein. In one embodiment, the present invention may be used to target a gene involved in, associated with, or causative of dominant genetic disorders such as, for example, SCN or CyN. In some embodiments, the dominant genetic disorder is SCN or CyN. In some embodiments, the target gene is the ELANE gene (Entrez Gene, gene ID No: 335).

### CRISPR Nucleases and PAM Recognition

In some embodiments, the sequence specific nuclease is selected from CRISPR nucleases, or a functional variant thereof. In some embodiments, the sequence specific nuclease is an RNA guided DNA nuclease. In such embodiments, the RNA sequence which guides the RNA guided DNA nuclease (e.g., Cpf1) binds to and/or directs the RNA guided DNA nuclease to the sequence comprising at least one nucleotide which differs between a mutant allele and its counterpart functional allele (e.g., SNP). In some embodiments, the CRISPR complex does not further comprise a tracrRNA. In a non-limiting example, in which the RNA guided DNA nuclease is a CRISPR protein, the at least one nucleotide which differs between the dominant mutant allele and the functional allele may be within the PAM site and/or proximal to the PAM site within the region that the RNA molecule is designed to hybridize to. A skilled artisan will appreciate that RNA molecules can be engineered to bind to a target of choice in a genome by commonly known methods in the art.

In embodiments of the present invention, a type II CRISPR system utilizes a mature crRNA:tracrRNA complex directs a CRISPR nuclease, e.g. Cas9, to the target DNA via Watson-Crick base-pairing between the crRNA and the protospacer on the target DNA next to the protospacer adjacent motif (PAM), an additional requirement for target recognition. The CRISPR nuclease then mediates cleavage of target DNA to create a double-stranded break within the protospacer. A skilled artisan will appreciate that each of the engineered RNA molecule of the present invention is further designed such as to associate with a target genomic DNA sequence of interest next to a protospacer adjacent motif (PAM), e.g., a PAM matching the sequence relevant for the type of CRISPR nuclease utilized, such as for a non-limiting example, NGG or NAG, wherein “N” is any nucleobase, for Streptococcus pyogenes Cas9 WT (SpCAS9); NNGRRT for Staphylococcus aureus (SaCas9); NNNVRYM for Jejuni Cas9 WT; NGAN or NGNG for SpCas9-VQR variant; NGCG for SpCas9-VRER variant; NGAG for SpCas9-EQR variant; NNNNGATT for Neisseria meningitidis (NmCas9); or TTTV for Cpf1. RNA molecules of the present invention are each designed to form complexes in conjunction with one or more different CRISPR nucleases and designed to target polynucleotide sequences of interest utilizing one or more different PAM sequences respective to the CRISPR nuclease utilized.

In some embodiments, an RNA-guided DNA nuclease e.g., a CRISPR nuclease, may be used to cause a DNA break at a desired location in the genome of a cell. The most commonly used RNA-guided DNA nucleases are derived from CRISPR systems, however, other RNA-guided DNA nucleases are also contemplated for use in the genome editing compositions and methods described herein. For instance, see U.S. Patent Publication No. 2015-0211023, incorporated herein by reference.

CRISPR systems that may be used in the practice of the invention vary greatly. CRISPR systems can be a type I, a type II, type III, or type V system. Non-limiting examples of suitable CRISPR proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas12a, Cas12b, Cas12c, Cas12d, Cas12d, Casl Od, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Cszl, Csx15, Csf1, Csf2, Csf3, Csf4, and Cul966. (See, e.g., Koonin 2017).

In some embodiments, the RNA-guided DNA nuclease is a CRISPR nuclease derived from a type II CRISPR system (e.g., Cas9). The CRISPR nuclease may be derived from Streptococcus pyogenes, Streptococcus thermophilus, Streptococcus sp., Staphylococcus aureus, Neisseria meningitidis, Treponema denticola, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Francisella cf. novicida Fx1, Alicyclobacillus acidoterrestris, Oleiphilus sp., Bacterium CG09_39_24, Deltaproteobacteria bacterium, or any species which encodes a CRISPR nuclease with a known PAM sequence. CRISPR nucleases encoded by uncultured bacteria may also be used in the context of the invention. (See Burstein et al. Nature, 2017). Variants of CRIPSR proteins having known PAM sequences e.g., SpCas9 D1135E variant, SpCas9 VQR variant, SpCas9 EQR variant, or SpCas9 VRER variant may also be used in the context of the invention.

In embodiments of the present invention, the CRISPR nuclease has cleavage activity when used with an RNA molecule comprising a guide sequence portion having 21-30, or 21-22, nucleotides and may be derived from any of the species recited above. A specific example of a CRISPR nuclease which has cleavage activity when used with an RNA molecule comprising a guide sequence portion having 21 nucleotides is described in Mojica, F. J., et al. (1993). A specific example of a CRISPR nuclease which has cleavage activity when used with an RNA molecule comprising a guide sequence portion having 22 nucleotides is described in Kim, E. et al. (2017). Mojica, F. J., et al. (1993) and Kim, E. et al. (2017) in their entirety and/or for the specific description CRISPR nucleases, are incorporated herein by reference. In such embodiments, the CRISPR nuclease may be engineered and/or non-naturally occurring.

Thus, an RNA guided DNA nuclease of a CRISPR system, such as a Cas9 protein or modified Cas9 or homolog or ortholog of Cas9, or other RNA guided DNA nucleases belonging to other types of CRISPR systems, such as Cpf1 and its homologs and orthologs, may be used in the compositions of the present invention.

In certain embodiments, the CRIPSR nuclease may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some cases, the cell does not naturally produce Cas protein and is genetically engineered to produce a Cas protein.

In some embodiments, the CRISPR nuclease is Cpf1. Cpf1 is a single RNA-guided endonuclease which utilizes a T-rich protospacer-adjacent motif. Cpf1 cleaves DNA via a staggered DNA double-stranded break. Two Cpf1 enzymes from Acidaminococcus and Lachnospiraceae have been shown to carry out efficient genome-editing activity in human cells. (See Zetsche et al. (2015) Cell.).

Thus, an RNA guided DNA nuclease of a Type II CRISPR System, such as a Cas9 protein or modified Cas9 or homologs, orthologues, or variants of Cas9, or other RNA guided DNA nucleases belonging to other types of CRISPR systems, such as Cpf1 and its homologs, orthologues, or variants, may be used in the present invention.

In some embodiments, the guide molecule comprises one or more chemical modifications which imparts a new or improved property (e.g., improved stability from degradation, improved hybridization energetics, or improved binding properties with an RNA guided DNA nuclease). Suitable chemical modifications include, but are not limited to one or more of: modified bases, modified sugar moieties, or modified inter-nucleoside linkages. Non-limiting examples of suitable chemical modifications include: 4-acetylcytidine, 5-(carboxyhydroxymethyl)uridine, 2′-O-methy cytidine, 5-carboxymethylaminomethyl-2-thiouridine, 5-carboxymethylaminomethyluridine, dihydrouridine, 2′-O-methylpseudouridine, “beta, D-galactosylqueuosine”, 2′-O-methylguanosine, inosine, N6-isopentenyladenosine, 1-methyladenosine, 1-methylpseudouridine, 1-methylguanosine, 1-methylinosine, “2,2-dimethylguanosine”, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, “beta, D-mannosylqueuosine”, 5-methoxycarbonylmethyl-2-thiouridine, 5-methoxycarbonylmethyluridine, 5-methoxyuridine, 2-methylthio-N6-isopentenyladenosine, N-((9-beta-D-ribofuranosyl-2-methylthiopurine-6-yl)carbamoyl)threonine, N-((9-beta-D-ribofuranosylpurine-6-yl)N-methylcarbamoyl)threonine, uridine-5-oxyacetic acid-methylester, uridine-5-oxyacetic acid, wybutoxosine, queuosine 2-thiocytidine, methyl-2-thiouridine, 2-thiouridine, 4-thiouridine, 5-methyluridine, N-((9-beta-D-ribofuranosylpurine-6-yl)-carbamoyl)threonine, 2′-O-methyl-5-methyluridine, 2′-O-methyluridine, wybutosine, “3-(3-amino-3-carboxy-propyl)uridine, (acp3)u”, 2′-O-methyl (M), 3′-phosphorothioate (MS), 3′-thioPACE (MSP), pseudouridine, or 1-methyl pseudo-uridine. Each possibility represents a separate embodiment of the present invention.

Further non-limiting examples of suitable chemical modifications include: m¹A (1-methyladenosine); m²A (2-methyladenosine); Am (2′-O-methyladenosine); ms² m⁶A (2-methylthio-N⁶-methyladenosine); i⁶A (N⁶-isopentenyladenosine); ms²i6A (2-methylthio-N⁶isopentenyladeno sine); io⁶A (N⁶-(cis-hydroxyisopentenyl)adenosine); ms²io⁶A (2-methylthio-N⁶-(cis-hydroxyisopentenyl) adenosine); g⁶A (N⁶-glycinylcarbamoyladenosine); t⁶A (N⁶-threonylcarbamoyladenosine); ms²t⁶A (2-methylthio-N⁶-threonyl carbamoyladenosine); m⁶t⁶A (N⁶-methyl-N⁶-threonylcarbamoyladenosine); hn⁶A (N⁶-hydroxynorvalylcarbamoyladenosine); ms²hn⁶A (2-methylthio-N⁶-hydroxynorvalyl carbamoyladenosine); Ar(p) (2′-O-ribosyladenosine (phosphate)); I (inosine); m¹I (1-methylinosine); m¹Im (1,2′-O-dimethylinosine); m³C (3-methylcytidine); Cm (2′-O-methylcytidine); s²C (2-thiocytidine); ac⁴C (N⁴-acetylcytidine); f⁵C (5-formylcytidine); m⁵Cm (5,2′-O-dimethylcytidine); ac⁴Cm (N⁴-acetyl-2′-O-methylcytidine); k²C (lysidine); m¹G (1-methylguanosine); m²G (N²-methylguanosine); m⁷G (7-methylguanosine); Gm (2′-O-methylguanosine); m² 2 G (N²,N²-dimethylguanosine); m²Gm (N^{2,2}′-O-dimethylguanosine); m²₂Gm (N²,N², 2′-O-trimethylguanosine); Gr(p) (2′-O-ribosylguanosine (phosphate)); yW (wybutosine); o₂yW (peroxywybutosine); OHyW (hydroxywybutosine); OHyW* (undermodified hydroxywybutosine); imG (wyosine); mimG (methylwyosine); Q (queuosine); oQ (epoxyqueuosine); galQ (galactosyl-queuosine); manQ (mannosyl-queuosine); preQ₀ (7-cyano-7-deazaguanosine); preQ₁ (7-aminomethyl-7-deazaguanosine); (archaeosine); D (dihydrouridine); m⁵Um (5,2′-O-dimethyluridine); s⁴U (4-thiouridine); m⁵s²U (5-methyl-2-thiouridine); s²Um (2-thio-2′-O-methyluridine); acp³U (3-(3-amino-3-carboxypropyl)uridine); ho⁵U (5-hydroxyuridine); mo⁵U (5-methoxyuridine); cmo⁵U (uridine 5-oxyacetic acid); mcmo⁵U (uridine 5-oxyacetic acid methyl ester); chm⁵U (5-(carboxyhydroxymethyl)uridine)); mchm⁵U (5-(carboxyhydroxymethyl)uridine methyl ester); mcm⁵U (5-methoxycarbonylmethyluridine); mcm⁵Um (5-methoxycarbonylmethyl-2′-O-methyluridine); mcm⁵s²U (5-methoxycarbonylmethyl-2-thiouridine); nm⁵S²U (5-aminomethyl-2-thiouridine); mnm⁵U (5-methylaminomethyluridine); mnm⁵s²U (5-methylaminomethyl-2-thiouridine); mnm⁵se²U (5-methylaminomethyl-2-selenouridine); ncm⁵U (5-carbamoylmethyluridine); ncm⁵Um (5-carbamoylmethyl-2′-O-methyluridine); cmnm⁵U (5-carboxymethylaminomethyluridine); cmnm⁵Um (5-carboxymethylaminomethyl-2′-O-methyluridine); cmmm⁵s²U (5-carboxymethylaminomethyl-2-thiouridine); dimethyladenosine); Im (2′-O-methylinosine); m⁴C (N⁴-methylcytidine); m⁴Cm (N⁴,2′-O-dimethylcytidine); hm⁵C (5-hydroxymethylcytidine); m³U (3-methyluridine); cm⁵U (5-carboxymethyluridine); m⁶Am (N⁶,2′-O-dimethyladenosine); m⁶₂Am (N⁶,N⁶,O-2′-trimethyladenosine); m^{2,7}G (N²,7-dimethylguanosine); m2,2,7G (N²,N²,7-trimethylguanosine); m³Um (3,2′-O-dimethyluridine); m⁵D (5-methyldihydrouridine); f⁵Cm (5-formyl-2′-O-methylcytidine); m¹Gm (1,2′-O-dimethylguanosine); m¹Am (1,2′-O-dimethyladenosine); τm⁵U (5-taurinomethyluridine); τm⁵s²U (5-taurinomethyl-2-thiouridine)); imG-14 (4-demethylwyosine); imG2 (isowyosine); or ac⁶A (N⁶-acetyladenosine). Each possibility represents a separate embodiment of the present invention. (See e.g. U.S. Pat. No. 9,750,824).

In embodiments of the present invention, the CRISPR nuclease has greater cleavage activity when used with an RNA molecule comprising a guide sequence portion having 21-23 nucleotides, compared to its cleavage activity when used with an RNA molecule comprising a guide sequence portion having 20 or fewer nucleotides, and/or 24 or more nucleotides. In embodiments of the present invention, the CRISPR nuclease has greater cleavage activity when used with an RNA molecule comprising a guide sequence portion having 21-22 nucleotides, compared to its cleavage activity when used with an RNA molecule comprising a guide sequence portion having 20 or fewer nucleotides, and/or 23 or more nucleotides. In an embodiment, the CRISPR nuclease has its greatest cleavage activity when used with an RNA molecule comprising a guide sequence portion having 22 nucleotides.

In an embodiment, such a CRISPR nuclease has at least 95% identity to the amino acid sequence as set forth in SEQ ID NO: 3789 or the sequence encoding the CRISPR nuclease has at least a 95% sequence identity to SEQ ID NO: 3790 or SEQ ID NO: 3791.

Guide Sequences which Specifically Target a Mutant Allele

A given gene may contain thousands of SNPs. Utilizing a 24 base pair target window for targeting each SNP in a gene would require hundreds of thousands of guide sequences. Any given guide sequence when utilized to target a SNP may result in degradation of the guide sequence, limited activity, no activity, or off-target effects. Accordingly, suitable guide sequences are necessary for targeting a given gene. By the present invention, a novel set of guide sequences have been identified for knocking out expression of a mutated protein, inactivating a mutant ELANE gene allele, and treating SCN or CyN.

The present disclosure provides guide sequences capable of specifically targeting a mutant allele for inactivation while leaving the functional allele unmodified. The guide sequences of the present invention are designed to, and are most likely to, specifically differentiate between a mutant allele and a functional allele. Of all possible guide sequences which target a mutant allele desired to be inactivated, the specific guide sequences disclosed herein are specifically effective to function with the disclosed embodiments.

Briefly, the guide sequences may have properties as follows: (1) target a heterozygous SNP/insertion/deletion/indel with a high prevalence in the general population, in a specific ethnic population or in a patient population is above 1% and the SNP/insertion/deletion/indel heterozygosity rate in the same population is above 1%; (2) target a location of a SNP/insertion/deletion/indel proximal to a portion of the gene e.g., within 5k bases of any portion of the gene, for example, a promoter, a UTR, an exon or an intron; and (3) target a mutant allele using an RNA molecule which targets a founder or common pathogenic mutations for the disease/gene. In some embodiments, the prevalence of the SNP/insertion/deletion/indel in the general population, in a specific ethnic population or in a patient population is above 1%, 2%, 3%, 4%, 5%, 6%, 7%, 8%, 9%, 10%, 11%, 12%, 13%, 14%, or 15% and the SNP/insertion/deletion/indel heterozygosity rate in the same population is above 1%, 2%, 3%, 4%, 5%, 6%, 7%, 8%, 9%, 10%, 11%, 12%, 13%, 14%, or 15%. Each possibility represents a separate embodiment and may be combined at will.

For each gene, according to SNP/insertion/deletion/indel any one of the following strategies may be used to deactivate the mutant allele: (1) Knockout strategy using one RNA molecule—one RNA molecule is utilized to direct a CRISPR nuclease to a mutant allele and create a double-strand break (DSB) leading to formation of a frameshift mutation in an exon or in a splice site region of the mutant allele; (2) Knockout strategy using two RNA molecules—a first RNA molecule targets a region in the promoter or an upstream region of a mutant allele and another RNA molecule targets downstream of the first RNA molecule in a promoter, exon, or intron of the mutant allele; (3) Exon(s) skipping strategy—one RNA molecule may be used to target a CRISPR nuclease to a splice site region, either at the 5′end of an intron (donor sequence) or the 3′ end of an intron (acceptor sequence), in order to destroy the splice site. Alternatively, two RNA molecules may be utilized such that a first RNA molecule targets an upstream region of an exon and a second RNA molecule targets a region downstream of the first RNA molecule, thereby excising the exon(s). Based on the locations of identified SNPs/insertions/deletions/indels for each mutant allele, any one of, or a combination of, the above-mentioned methods to deactivate the mutant allele may be utilized.

When two RNA molecules are used, guide sequences may target two SNPs such that the first SNP is upstream of exon 1 e.g., within the 5′ untranslated region, or within the promoter or within the first 2 kilobases 5′ of the transcription start site, and the second SNP is downstream of the first SNP e.g., within the first 2 kilobases 5′ of the transcription start site, or within intron 1, 2 or 3, or within exon 1, exon 2, or exon 3.

Guide sequences of the present invention may target a SNP in the upstream portion of the targeted gene, preferably upstream of the last exon of the targeted gene. Guide sequences may target a SNP upstream to exon 1, for example within the 5′ untranslated region, or within the promoter or within the first 4-5 kilobases 5′ of the transcription start site.

Guide sequences of the present invention may also target a SNP within close proximity (e.g., within 50 basepairs, more preferably with 20 basepairs) to a known protospacer adjacent motif (PAM) site.

Guide sequences of the present invention also may target: (1) a heterozygous SNP for the targeted gene; (2) a heterozygous SNPs upstream and downstream of the gene; (3) a SNPs with a prevalence of the SNP/insertion/deletion/indel in the general population, in a specific ethnic population, or in a patient population above 1%; (4) have a guanine-cytosine content of greater than 30% and less than 85%; (5) have no repeat of 4 or more thymine/uracil or 8 or more guanine, cytosine, or adenine; (6) having no off-target identified by off-target analysis; and (7) preferably target Exons over Introns or be upstream of a SNP rather than downstream of a SNP.

In embodiments of the present invention, the SNP may be upstream or downstream of the gene. In embodiments of the present invention, the SNP is within 3000 base pairs upstream or downstream of the gene.

The at least one nucleotide which differs between the mutant allele and the functional allele, may be upstream, downstream or within the sequence of the disease-causing mutation of the gene of interest. The at least one nucleotide which differs between the mutant allele and the functional allele, may be within an exon or within an intron of the gene of interest. In some embodiments, the at least one nucleotide which differs between the mutant allele and the functional allele is within an exon of the gene of interest. In some embodiments, the at least one nucleotide which differs between the mutant allele and the functional allele is within an intron or an exon of the gene of interest, in close proximity to a splice site between the intron and the exon e.g., 1, 2, 3, 4, 5, 6, 7, 8, 9, 10, 11, 12, 13, 14, 15, 16, 17, 18, 19, or 20 nucleotides upstream or downstream to the splice site.

In some embodiments, the at least one nucleotide is a single nucleotide polymorphisms (SNPs). In some embodiments, each of the nucleotide variants of the SNP may be expressed in the mutant allele. In some embodiments, the SNP may be a founder or common pathogenic mutation.

Guide sequences may target a SNP which has both (1) a high prevalence in the general population e.g., above 1% in the population; and (2) a high heterozygosity rate in the population, e.g., above 1%. Guide sequences may target a SNP that is globally distributed. A SNP may be a founder or common pathogenic mutation. In some embodiments, the prevalence in the general population is above 1%, 2%, 3%, 4%, 5%, 6%, 7%, 8%, 9%, 10%, 11%, 12%, 13%, 14%, or 15%. Each possibility represents a separate embodiment. In some embodiments, the heterozygosity rate in the population is above 1%, 2%, 3%, 4%, 5%, 6%, 7%, 8%, 9%, 10%, 11%, 12%, 13%, 14%, or 15%. Each possibility represents a separate embodiment.

In some embodiments, the at least one nucleotide which differs between the mutant allele and the functional allele is linked to/co-exists with the disease-causing mutation in high prevalence in a population. In such embodiments, “high prevalence” refers to at least 92%, 93%, 94%, 95%, 96%, 97%, 98%, 99%, or 100%. Each possibility represents a separate embodiment of the present invention. In one embodiment, the at least one nucleotide which differs between the mutant allele and the functional allele, is a disease-associated mutation. In some embodiments, the SNP is highly prevalent in the population. In such embodiments, “highly prevalent” refers to at least 10%, 11%, 12%, 13%, 14%, 15%, 20%, 30%, 40%, 50%, 60%, or 70% of a population. Each possibility represents a separate embodiment of the present invention.

Guide sequences of the present invention may satisfy any one of the above criteria and are most likely to differentiate between a mutant allele from its corresponding functional allele.

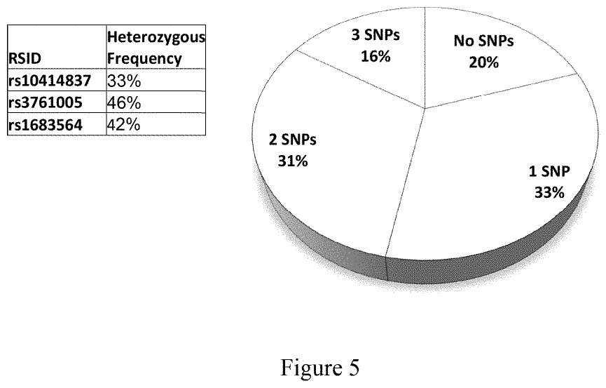

FIG. 5 discloses the heterogenicity of selections of ELANE SNPs in the human population.

Embodiments of the present invention may include excising the promoter region from an upstream SNP position until intron 4. In an embodiment, a first guide sequence targets a specific sequence of a heterozygous SNP position in an upstream region of the mutant allele (strategy 1a—rs10414837, strategy 1b—rs3761005) and a second guide sequence targets a sequence in intron 4 which is common to two alleles of the gene. (FIG. 1).

Embodiments of the present invention may include excising from intron 4 to regions downstream to the 3′ UTR. In an embodiment, a first guide sequence targets a specific sequence of a heterozygous SNP position in an upstream region of the mutant allele (strategy 2-rs1683564) and a second guide sequence targets a sequence in intron 4 which is common to two alleles of the gene.

Embodiments of the present invention excising from intron 4 to regions downstream to the 3′ UTR (FIG. 4). The strategy is designed such as to specifically knock-out the disease-causing allele (‘mutant allele’), while leaving the healthy allele intact. Allele specific editing is achieved by using guides that target discriminating (heterozygous) SNP positions with relatively high heterozygosity frequency in the population.

### Delivery to Cells

It is understood that in the methods embodied, the RNA molecules and compositions described herein may be delivered to a target cell or subject by any suitable means. The following embodiments provide non-limiting examples of methods of delivery of the RNA molecules and composition of the present invention.

In some embodiments, RNA molecule compositions of the present invention may be targeted to any cell which contains and/or expresses a dominant negative allele, including any mammalian or plant cell. For example, in one embodiment the RNA molecule specifically targets a mutant ELANE allele and the target cell is a hepatocyte cell.

Any suitable viral vector system may be used to deliver nucleic acid compositions e.g., the RNA molecule compositions of the subject invention. Conventional viral and non-viral based gene transfer methods can be used to introduce nucleic acids and target tissues. In certain embodiments, nucleic acids are administered for in vivo or ex vivo gene therapy uses. Non-viral vector delivery systems include naked nucleic acid, and nucleic acid complexed with a delivery vehicle such as a liposome or poloxamer. For a review of gene therapy procedures, see Anderson (1992) Science 256:808-813; Nabel & Felgner (1993) TIBTECH 11:211-217; Mitani & Caskey (1993) TIBTECH 11:162-166; Dillon (1993) TIBTECH 11:167-175; Miller (1992) Nature 357:455-460; Van Brunt (1988) Biotechnology 6(10):1149-1154; Vigne (1995) Restorative Neurology and Neuroscience 8:35-36; Kremer & Perricaudet (1995) British Medical Bulletin 51(1):31-44; Haddada et al. (1995) in Current Topics in Microbiology and Immunology Doerfler and Bohm (eds.); and Yu et al. (1994) Gene Therapy 1:13-26.

Methods of non-viral delivery of nucleic acids and/or proteins include electroporation, lipofection, microinjection, biolistics, particle gun acceleration, virosomes, liposomes, immunoliposomes, lipid nanoparticles (LNPs), polycation or lipid:nucleic acid conjugates, artificial virions, and agent-enhanced uptake of nucleic acids or can be delivered to plant cells by bacteria or viruses (e.g., Agrobacterium, Rhizobium sp. NGR234, Sinorhizoboiummeliloti, Mesorhizobium loti, tobacco mosaic virus, potato virus X, cauliflower mosaic virus and cassava vein mosaic virus). (See, e.g., Chung et al. (2006) Trends Plant Sci. 11(1):1-4). Sonoporation using, e.g., the Sonitron 2000 system (Rich-Mar), can also be used for delivery of nucleic acids. Cationic-lipid mediated delivery of proteins and/or nucleic acids is also contemplated as an in vivo or in vitro delivery method. (See Zuris et al. (2015) Nat. Biotechnol. 33(1):73-80; see also Coelho et al. (2013) N. Engl. J. Med. 369, 819-829; Judge et al. (2006) Mol. Ther. 13, 494-505; and Basha et al. (2011) Mol. Ther. 19, 2186-2200).

Additional exemplary nucleic acid delivery systems include those provided by Amaxa® Biosystems (Cologne, Germany), Maxcyte, Inc. (Rockville, Md.), BTX Molecular Delivery Systems (Holliston, Mass.) and Copernicus Therapeutics Inc., (see, e.g., U.S. Pat. No. 6,008,336). Lipofection is described in e.g., U.S. Pat. Nos. 5,049,386, 4,946,787; and 4,897,355, and lipofection reagents are sold commercially (e.g., Transfectam™, Lipofectin™ and Lipofectamine™ RNAiMAX). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration).

The preparation of lipid:nucleic acid complexes, including targeted liposomes such as immunolipid complexes, is well known to one of skill in the art (See, e.g., Crystal (1995) Science 270:404-410; Blaese et al. (1995) Cancer Gene Ther. 2:291-297; Behr et al. (1994) Bioconjugate Chem. 5:382-389; Remy et al. (1994) Bioconjugate Chem. 5:647-654; Gao et al. (1995) Gene Therapy 2:710-722; Ahmad et al. (1992) Cancer Res. 52:4817-4820; U.S. Pat. Nos. 4,186,183, 4,217,344, 4,235,871, 4,261,975, 4,485,054, 4,501,728, 4,774,085, 4,837,028, and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (See MacDiarmid et al (2009) Nature Biotechnology 27(7):643).

The use of RNA or DNA viral based systems for viral mediated delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids include, but are not limited to, retroviral, lentivirus, adenoviral, adeno-associated, vaccinia and herpes simplex virus vectors for gene transfer.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (See, e.g., Buchschacher et al. (1992) J. Virol. 66:2731-2739; Johann et al. (1992) J. Virol. 66:1635-1640; Sommerfelt et al. (1990) Virol. 176:58-59; Wilson et al. (1989) J. Virol. 63:2374-2378; Miller et al. (1991) J. Virol. 65:2220-2224; PCT/US94/05700).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (Dunbar et al. (1995) Blood 85:3048-305; Kohn et al. (1995) Nat. Med. 1:1017-102; Malech et al. (1997) PNAS 94:22 12133-12138). PA317/pLASN was the first therapeutic vector used in a gene therapy trial. (Blaese et al. (1995). Transduction efficiencies of 50% or greater have been observed for MFG-S packaged vectors. (Ellem et al. (1997) Immunol Immunother. 44(1):10-20; Dranoff et al. (1997) Hum. Gene Ther. 1:111-2).

Packaging cells are used to form virus particles that are capable of infecting a host cell. Such cells include 293 cells, which package adenovirus, AAV, and Psi-2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additionally, AAV can be produced at clinical scale using baculovirus systems (see U.S. Pat. No. 7,479,554).

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al. (1995) Proc. Natl. Acad. Sci. USA 92:9747-9751, reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual patient, typically by systemic administration (e.g., intravitreal, intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid composition, and re-infused back into the subject organism (e.g., patient). Various cell types suitable for ex vivo transfection are well known to those of skill in the art (See, e.g., Freshney et al. (1994) Culture of Animal Cells, A Manual of Basic Technique, 3rd ed, and the references cited therein for a discussion of how to isolate and culture cells from patients).

Suitable cells include, but are not limited to, eukaryotic cells and/or cell lines. Non-limiting examples of such cells or cell lines generated from such cells include COS, CHO (e.g., CHO-S, CHO-K1, CHO-DG44, CHO-DUXB11, CHO-DUKX, CHOK1SV), VERO, MDCK, WI38, V79, B14AF28-G3, BHK, HaK, NSO, SP2/0-Ag14, HeLa, HEK293 (e.g., HEK293-F, HEK293-H, HEK293-T), perC6 cells, any plant cell (differentiated or undifferentiated), as well as insect cells such as Spodopterafugiperda (Sf), or fungal cells such as Saccharomyces, Pichia and Schizosaccharomyces. In certain embodiments, the cell line is a CHO-K1, MDCK or HEK293 cell line. Additionally, primary cells may be isolated and used ex vivo for reintroduction into the subject to be treated following treatment with a guided nuclease system (e.g. CRISPR/Cas). Suitable primary cells include peripheral blood mononuclear cells (PBMC), and other blood cell subsets such as, but not limited to, CD4+ T cells or CD8+ T cells. Suitable cells also include stem cells such as, by way of example, embryonic stem cells, induced pluripotent stem cells, hematopoietic stem cells (CD34+), neuronal stem cells and mesenchymal stem cells.

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-gamma, and TNF-alpha are known (as a non-limiting example see, Inaba et al., J. Exp. Med. 176:1693-1702 (1992)).

Stem cells are isolated for transduction and differentiation using known methods. For example, stem cells are isolated from bone marrow cells by panning the bone marrow cells with antibodies which bind unwanted cells, such as CD4+ and CD8+(T cells), CD45+(panB cells), GR-1 (granulocytes), and Tad (differentiated antigen presenting cells) (as a non-limiting example see Inaba et al. (1992) J. Exp. Med. 176:1693-1702). Stem cells that have been modified may also be used in some embodiments.

Typically, the cells are administered in a pharmaceutical composition comprising at least one pharmaceutically-acceptable carrier. The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically-acceptable material, composition or vehicle, such as a liquid or solid filler, diluent, excipient, or solvent encapsulating material.

Any one of the RNA molecule compositions described herein is suitable for genome editing in post-mitotic cells or any cell which is not actively dividing, e.g., arrested cells. Examples of post-mitotic cells which may be edited using an RNA molecule composition of the present invention include, but are not limited to, a hepatocyte cell.

Vectors (e.g., retroviruses, liposomes, etc.) containing therapeutic nucleic acid compositions can also be administered directly to an organism for transduction of cells in vivo. Administration is by any of the routes normally used for introducing a molecule into ultimate contact with blood or tissue cells including, but not limited to, injection, infusion, topical application (e.g., eye drops and cream) and electroporation. Suitable methods of administering such nucleic acids are available and well known to those of skill in the art, and, although more than one route can be used to administer a particular composition, a particular route can often provide a more immediate and more effective reaction than another route. According to some embodiments, the composition is delivered via IV injection.

Vectors suitable for introduction of transgenes into immune cells (e.g., T-cells) include non-integrating lentivirus vectors. See, e.g., U.S. Patent Publication No. 2009-0117617.

Pharmaceutically acceptable carriers are determined in part by the particular composition being administered, as well as by the particular method used to administer the composition. Accordingly, there is a wide variety of suitable formulations of pharmaceutical compositions available, as described below (See, e.g., Remington's Pharmaceutical Sciences, 17th ed., 1989).

In accordance with some embodiments, there is provided an RNA molecule which binds to/associates with and/or directs the RNA guided DNA nuclease to a sequence comprising at least one nucleotide which differs between a mutant allele and a functional allele (e.g., SNP) of a gene of interest (i.e., a sequence of the mutant allele which is not present in the functional allele). The sequence may be within the disease associated mutation. The sequence may be upstream or downstream to the disease associated mutation. Any sequence difference between the mutant allele and the functional allele may be targeted by an RNA molecule of the present invention to inactivate the mutant allele, or otherwise disable its dominant disease-causing effects, while preserving the activity of the functional allele.

The disclosed compositions and methods may also be used in the manufacture of a medicament for treating dominant genetic disorders in a patient.

### Mechanisms of Action for Several Embodiments Disclosed Herein

Mutations in ELANE that were demonstrated to lead to SCN or CyN, mediate translation from alternative in frame ORF (open reading frame) that generate truncated N-terminus protein thus causing ER and protein misfolding stress.

Without being bound by any theory or mechanism, the instant invention may be utilized to apply a CRISPR nuclease to process the mutated pathologic ELANE allele and not the functional ELANE allele, such as to prevent expression of the mutated pathologic allele or to produce a truncated non-pathologic peptide from the mutated pathologic allele, or to repair/correct the mutated pathologic ELANE allele in order to prevent ameliorate or treat SCN or CyN.

Several alternative editing strategies utilizing SNPs located upstream and downstream to the ORF may be applied. The strategies include exclusion of the whole gene, truncation of the gene to exclude the C-terminus of the gene, and attenuation of the expression of the gene.

In some embodiments, two guides (e.g., guides disclosed in Table 1) may be utilized to remove the entire gene (i.e., exons 1, 2, 3, 4, and 5) to knockout the mutant protein. In some embodiments, a first guide RNA is utilized to mediate an allele specific DSB by targeting a SNP/WT sequence located upstream to the ORF of the mutated allele of the ELANE gene, and a second guide RNA may be utilized to mediate DSB in a SNP/WT sequence located in exon 5 or downstream to the mutated allele of the ELANE gene, or a sequence located in intron 4, 3′ UTR or downstream to the alleles of the ELANE gene, or a SNP/WT sequence located in intron 4 3′UTR or downstream to the alleles of the ELANE gene.

There are records of healthy individuals harboring frameshift mutation that result in gain of stop codon located till exon 3. Therefore, a potential strategy may be to truncate the mutated allele such that to include at most exons 1 till 3. In some embodiments, two guides (e.g., guides disclosed in Table 1) may be utilized to truncate the c-terminus of the mutated allele of the ELANE gene. In some embodiments, a first guide RNA may be utilized to mediate an allele specific DSB by targeting a SNP/WT sequence in exon 5 or downstream of the mutated allele, and a second guide RNA may be utilized to mediate DSB in a sequence located in intron 1, 2 or 3 of the ELANE gene, or a SNP/WT sequence. A peptide/protein encoded by the truncated mutated allele may exhibit no pathological effect. Alternatively, a nonsense-mediated mRNA decay may be triggered resulting in knockout of the expression of the mutated allele. Results may be verified by examining mRNA and protein expression.

In some embodiments, the expression of the mutated allele may be attenuated by excising elements from the proximal promoter and enhancer regions using the SNPs located upstream to the ORF. In a non-limiting example, a significant reduction may be achieved by excising most of the enhancer region by targeting a SNP.

### Examples of RNA Guide Sequences which Specifically Target Mutant Alleles of ELANE Gene

Although a large number of guide sequences can be designed to target a mutant allele, the nucleotide sequences described in Table 1 identified by SEQ ID NOs: 1-3751 below were specifically selected to effectively implement the methods set forth herein and to effectively discriminate between alleles.

Table 1 shows guide sequences designed for use as described in the embodiments above to associate with different SNPs within a sequence of a mutant ELANE allele. Each engineered guide molecule is further designed such as to associate with a target genomic DNA sequence of interest that lies next to a protospacer adjacent motif (PAM), e.g., a PAM matching the sequence NGG or NAG, where “N” is any nucleobase. The guide sequences were designed to work in conjunction with one or more different CRISPR nucleases, including, but not limited to, e.g. SpCas9WT (PAM SEQ: NGG), SpCas9.VQR.1 (PAM SEQ: NGAN), SpCas9.VQR.2 (PAM SEQ: NGNG), SpCas9.EQR (PAM SEQ: NGAG), SpCas9.VRER (PAM SEQ: NGCG), SaCas9WT (PAM SEQ: NNGRRT), NmCas9WT (PAM SEQ: NNNNGATT), Cpf1 (PAM SEQ: TTTV), or JeCas9WT (PAM SEQ: NNNVRYM). RNA molecules of the present invention are each designed to form complexes in conjunction with one or more different CRISPR nucleases and designed to target polynucleotide sequences of interest utilizing one or more different PAM sequences respective to the CRISPR nuclease utilized.

TABLE 1
Guide sequences designed to associate with the ELANE gene

| | SEQ ID | SEQ ID | SEQ ID |
|---|---|---|---|
| | NOs: of | NOs: of | NOs: of |
| | 20 base | 21 base | 22 base |
| Target | guides | guides | guides |
| 19:848798_T_C_rs10424470_REF | N/A | 1-48 | 49-98 |
| 19:848798_T_C_rs10424470_SNP | N/A | 3, 11, 16, | 51, 59, 64, |
| | | 22, 29, 41, | 77, 86, 90, |
| | | 45, 48, 99- | 93, 98, 139- |
| | | 138 | 180 |
| 19:849138_T_C_rs4807932_REF | N/A | | 181 |
| 19:849138_T_C_rs4807932_SNP | N/A | 182-185 | 181, 186- |
| | | | 195 |
| 19:849618_C_T_rs10414837_REF | N/A | 196-243 | 244-293 |
| 19:849618_C_T_rs10414837_SNP | N/A | 199, 202, | 244, 248, |
| | | 207, 217, | 251, 256, |
| | | 225, 228, | 258, 267, |
| | | 230, 235, | 275, 278, |
| | | 294-333 | 334-375 |
| 19:849688_CTTTTTTTTTT_C_rs376107533_REF | N/A | 376-382 | 383-391 |
| 19:849935_A_C_rs3761010_REF | N/A | 392-404 | 405-419 |
| 19:849935_A_C_rs3761010_SNP | N/A | | 411 |
| 19:849959_G_A_rs351108_SNP | N/A | | 420-422 |
| 19:850023_T_C_rs3761008_SNP | N/A | 423-424 | 425-428 |
| 19:850199_T_A_rs3826946_REF | N/A | 429-453 | 454-493 |
| 19:850199_T_A_rs3826946_SNP | N/A | 431-432, | 454, 457, |
| | | 434, 436, | 462, 471- |
| | | 440-441, | 472, 483, |
| | | 452, 494- | 489-490, |
| | | 511 | 512-543 |
| 19:850299_G_A_rs10413889_REF | N/A | 544-591 | 592-641 |
| 19:850299_G_A_rs10413889_SNP | N/A | 551, 559, | 599, 616, |
| | | 569-570, | 619, 623- |
| | | 574-575, | 624, 626, |
| | | 577, 579, | 628, 630, |
| | | 642-681 | 682-723 |
| 19:850574_G_A_rs3761007_REF | N/A | 724-771 | 772-821 |
| 19:850574_G_A_rs3761007_SNP | N/A | 726-727, | 774, 781, |
| | | 732-733, | 784, 794, |
| | | 736, 746, | 796, 806, |
| | | 759, 764, | 809, 814, |
| | | 822-861 | 862-903 |
| 19:850733_C_G_rs10409474_REF | N/A | 904-951 | 952-1001 |
| 19:850733_C_G_rs10409474_SNP | N/A | 920-921, | 968-970, |
| | | 925-926, | 975, 983, |
| | | 934, 939, | 987, 989, |
| | | 943, 950, | 1000, 1042- |
| | | 1002-1041 | 1083 |
| 19:850793_T_A_rs3761005_REF | N/A | 1084-1131 | 1132-1181 |
| 19:850793_T_A_rs3761005_SNP | N/A | 1088, 1095, | 1136, 1142, |
| | | 1099, 1101, | 1144, 1148, |
| | | 1107, 1111, | 1156, 1160, |
| | | 1119-1120, | 1164, 1170, |
| | | 1182-1221 | 1222-1263 |
| 19:851401_T_G_rs351107_SNP | N/A | 1264-1311 | 1312-1361 |
| 19:851563_G_A_rs3761001_REF | N/A | 1362-1390 | 1391-1421 |
| 19:852104_C_A_rs740021_REF | N/A | 1422-1426 | 1427-1437 |
| 19:852104_C_A_rs740021_SNP | N/A | 1422-1423, | 1427, 1429, |
| | | 1426 | 1434-1435 |
| 19:852594_AGG_A_rs781452480_REF | N/A | 1438-1454 | 1455-1471 |
| 19:852594_AGG_A_rs781452480_SNP | N/A | 1454 | 1469, 1472 |
| 19:852594_AG_A_rs371057361_REF | N/A | 1438-1454 | 1455-1471 |
| 19:852594_A_G_rs570466264_REF | N/A | 1438-1454 | 1455-1471 |
| 19:852594_A_G_rs570466264_SNP | N/A | 1438-1439, | 1455-1457, |
| | | 1446, 1450, | 1464, 1476- |
| | | 1473-1475 | 1478 |
| 19:852606_T_G_rs1041904080_REF | N/A | 1479-1493 | 1472, 1494- |
| | | | 1509 |
| 19:852606_T_G_rs1041904080_SNP | N/A | 1485-1486, | 1496, 1500, |
| | | 1492-1493, | 1508-1509, |
| | | 1510-1512 | 1513-1517 |
| 19:855162_T_A_rs7250194_SNP | N/A | 1518-1519 | 1520-1521 |
| 19:855966_C_A_rs17216649_REF | N/A | 1522-1567 | 1568-1617 |
| 19:855966_C_A_rs17216649_SNP | N/A | 1534, 1537, | 1581, 1584, |
| | | 1540, 1557- | 1587, 1592, |
| | | 1558, 1561, | 1596, 1607, |
| | | 1565, 1567, | 1615, 1617, |
| | | 1618-1657 | 1658-1699 |
| 19:856416_T_TA_rs199720952_REF | N/A | 1700-1716 | 1717-1733 |
| 19:856416_T_TA_rs199720952_SNP | N/A | 1703 | 1718 |
| 19:856482_C_T_rs6510983_REF | N/A | 1734-1752 | 1753-1775 |
| 19:856482_C_T_s6510983_SNP | N/A | 1734-1735, | 1753, 1757, |
| | | 1738, 1740, | 1759, 1772, |
| | | 1776-1794 | 1795-1815 |
| 19:856530_G_A_rs17223066_REF | N/A | 1816-1818 | 1819-1823 |
| 19:856530_G_A_rs17223066_SNP | N/A | 1816-1817, | 1820-1821, |
| | | 1824-1832 | 1823, 1833- |
| | | | 1842 |
| 19:856783_T_A_rs7255385_REF | N/A | 1843-1847 | 1848-1854 |
| 19:857215_G_A_rs9749274_REF | N/A | 1855-1867 | 1868-1882 |
| 19:857215_G_A_rs9749274_SNP | N/A | 1860, 1862- | 1874-1875, |
| | | 1863, 1865, | 1877, 1880, |
| | | 1883-1887 | 1888-1894 |
| 19:857444_C_T_rs111361200_SNP | N/A | | 1895 |
| 19:857848_CA_C_rs112639467_REF | N/A | 1896-1900 | 1901-1907 |
| 19:857848_C_CA_rs141213775_REF | N/A | 1896-1900 | 1901-1907 |
| 19:858296_C_A_rs28591229_REF | N/A | 1908-1953 | 1954-2001 |
| 19:858296_C_A_rs28591229_SNP | N/A | 1917, 1920, | 1983, 1985, |
| | | 1937, 1939, | 1989-1991, |
| | | 1943-1944, | 1999, 2042- |
| | | 2002-2041 | 2083 |
| 19:858376_C_T_rs10469327_REF | N/A | 2084-2131 | 2132-2181 |
| 19:858376_C_T_rs10469327_SNP | N/A | 2085-2086, | 2132, 2134- |
| | | 2092, 2105, | 2135, 2141, |
| | | 2110, 2113, | 2159, 2162, |
| | | 2118-2119, | 2168, 2179, |
| | | 2182-2221 | 2222-2263 |
| 19:859067_GA_G_rs3834645_REF | N/A | 2264-2307 | 2308-2353 |
| 19:859067_GA_G_rs3834645_SNP | N/A | 2274, 2287, | 2319, 2333, |
| | | 2293, 2300, | 2339, 2351, |
| | | 2354-2392 | 2393-2433 |
| 19:859214_C_A_rs1683564_REF | N/A | 2434-2481 | 2482-2531 |
| 19:859214_C_A_rs1683564_SNP | N/A | 2435-2436, | 2483, 2490, |
| | | 2442, 2444, | 2499, 2504, |
| | | 2451, 2456, | 2513, 2519, |
| | | 2465, 2471, | 2521, 2524, |
| | | 2532-2571 | 2572-2613 |
| 19:859368_G_T_rs71335276_REF | N/A | 2614-2661 | 2662-2711 |
| 19:859368_G_T_rs71335276_SNP | N/A | 2616, 2624, | 2672, 2676, |
| | | 2627, 2633, | 2682, 2685, |
| | | 2636, 2641, | 2687, 2691, |
| | | 2647, 2654, | 2697, 2704, |
| | | 2712-2751 | 2752-2793 |
| 19:859831_T_G_rs8107095_REF | N/A | 2794-2841 | 2842-2891 |
| 19:859831_T_G_rs8107095_SNP | N/A | 2794-2796, | 2842-2843, |
| | | 2801-2802, | 2848, 2850- |
| | | 2804, 2826, | 2851, 2875, |
| | | 2835, 2892- | 2883, 2885, |
| | | 2917 | 2918-2953 |
| 19:855795-855957 | 2954- | 3218-3483 | 3484-3751 |
| (Intron 4) | 3217 | | |

The indicated locations listed in column 1 of the Table 1 are based on gnomAD v3 database and UCSC Genome Browser assembly ID: hg38, Sequencing/Assembly provider ID: Genome Reference Consortium Human GRCh38.p12 (GCA_000001405.27). Assembly date: December 2013 initial release; December 2017 patch release 12.The SNP details are indicated by the listed SNP ID NOs. (“rs numbers”), which are based onthe NCBI 2018 database of Single Nucleotide Polymorphisms (dbSNP)). The indicated DNA mutations are associated with Transcript Consequence NM_001972 as obtained from NCBI RefSeq genes.

For the foregoing embodiments, each embodiment disclosed herein is contemplated as being applicable to each of the other disclosed embodiment. For example, it is understood that any of the RNA molecules or compositions of the present invention may be utilized in any of the methods of the present invention.

As used herein, all headings are simply for organization and are not intended to limit the disclosure in any manner. The content of any individual section may be equally applicable to all sections.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

Further. the examples herein below disclose methods utilizing SpCas9 and guide sequences suitable to target the SpCas9 to the disclosed SNP positions. The examples demonstrate the feasibility of the strategies disclosed. A person having ordinary skill in the art would understand that the same guides sequences may be used with different CRISPR nuclease to target the disclosed SNPs to apply each of the specified strategies. Further, different guide sequences that target other CRISPR nucleases to the same SNPs may be used together with the other nucleases to apply each of the specified strategies.

### EXPERIMENTAL DETAILS

### Example 1: Screening Guide Sequences Suitable to Work in Conjunction with SpCas9 and Targeting SNPs and Sequences Complying with the Disclosed Strategies

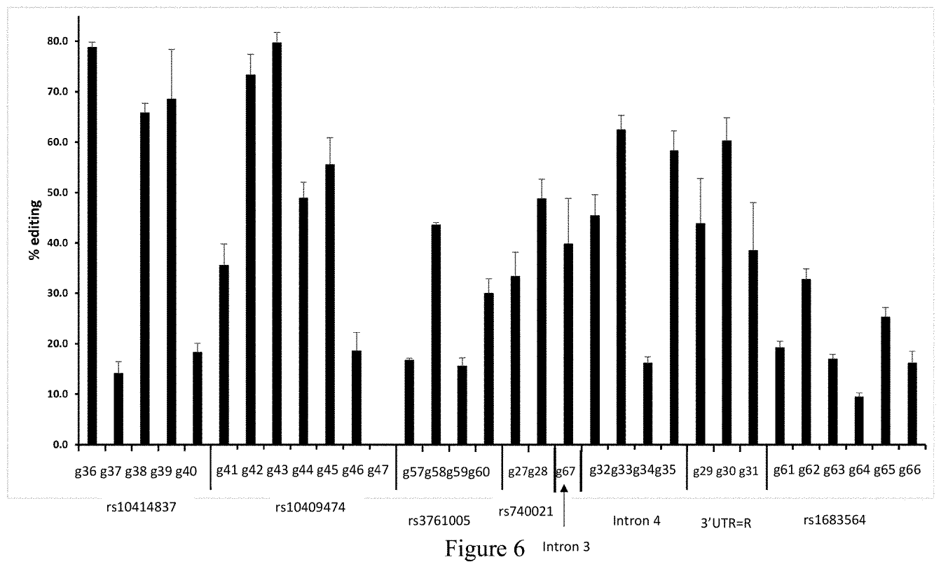

HeLa cells were seeded into 96 well-plate (3K/well). 24 hours later, cells were co-transfected with either 65 ng of WT-Cas9 or Dead-Cas9 and 20 ng of a gRNA plasmid, identified as g36 through g66, targeting different regions and SNPs in ELANE using Turbofect reagent (Thermo Scientific). 12 hours later, fresh media was added, and 72 hours post-transfection, genomic DNA was extracted, and the expected region targeted by the Cas9 was amplified. The product size was analyzed by capillary electrophoresis with a DNA ladder standard. The intensity of the bands was analyzed using the Peak Scanner software v1.0. The percent of editing was calculated according the following formula: 100%−(Intensity not edited band/Intensity total bands)*100. FIG. 6 represents the mean activity of each gRNA following subtraction of the Dead-Cas9 background activity±SD of three independent experiments.

TABLE 2
guides sg36 through sg66 of Example 1 as identified by SEQ ID NO.

| | Example 1 | SEQ ID | |
|---|---|---|---|
| Guide sequence | gID | NO: | SNP location |
| UAGGGGUGUUAUGGUCACAG | 836 | 3752 | upstream -2590 bp |
| CACAGCGGGUGUAGACUCCG | 937 | 3753 | upstream -2590 bp |
| ACAGCGGGUGUAGACUCCGA | 938 | 3754 | upstream -2590 bp |
| CAGCGGGUGUAGACUCCGAG | 839 | 3755 | upstream -2590 bp |
| AGCGGGUGUAGACUCCGAGG | 940 | 3756 | upstream -2590 bp |
| CCGUUGCAGCUGGAACAUCG | 841 | 3757 | upstream -1475 bp |
| CGUUGCAGCUGGAACAUCGU | 942 | 3758 | upstream -1475 bp |
| GUUGCAGCUGGAACAUCGUG | 943 | 3759 | upstream -1475 bp |
| UUGCAGCUGGAACAUCGUGG | 844 | 3760 | upstream -1475 bp |
| CUGGAACAUCGUGGGGGAGA | 845 | 3761 | upstream -1475 bp |
| UGGAACAUCGUGGGGGAGAU | 946 | 3762 | upstream -1475 bp |
| AUCGUGGGGGAGAUGGGAAG | 947 | 3763 | upstream -1475 bp |
| GGAGUCCCAGCUGCGGGAAA | 957 | 3764 | upstream -1415 bp |
| GCUGCGGGAAAGGGAUUCCC | 858 | 3765 | upstream -1415 bp |
| GGGAAUCCCUUUCCCGCAGC | 859 | 3766 | upstream -1415 bp |
| GGAAUCCCUUUCCCGCAGCU | 860 | 3767 | upstream -1415 bp |
| CAAAUGUCAGAUAAUCAAUG | 927 | 3768 | upstream |
| AAAUGUCAGAUAAUCAAUGU | 828 | 3769 | upstream |
| ACCAAGGCUCAGGGCGUUGG | 867 | 3770 | Int3 |
| CCUGUUGCUGCAGUCCGGGC | 932 | 3771 | Int4 |
| CCAGCCCGGACUGCAGCAAC | 933 | 3772 | Int4 |
| UCCCUCCUAGGGUCUAGCCA | 834 | 3773 | Int4 |
| AGUCCGGGCUGGGAGCGGGU | 935 | 3774 | Int4 |
| AUGUUUAUUGUGCCAGAUGC | 829 | 3775 | 3UTR |
| GUGGGCAGCUGAGGUGACCC | 930 | 3776 | 3UTR |
| CACCCACACUCUCCAGCAUC | 831 | 3777 | 3UTR |
| UGUCAAGCCCCAGAGGCCAC | 861 | 3778 | downstream +2968 bp |
| GUCAAGCCCCAGAGGCCACA | 862 | 3779 | downstream +2968 bp |
| GUCUCUGUCCCUGUGGCCUC | 863 | 3780 | downstream +2968 bp |
| UCUCUGUCCCUGUGGCCUCU | 864 | 3781 | downstream +2968 bp |
| CUCUGUCCCUGUGGCCUCUG | 865 | 3782 | downstream +2968 bp |
| UGUCAAGCCCCAGAGGCCAC | g66 | 3778 | downstream +2968 bp |

### Example 2: Demonstrating the Feasibility of the Excision Strategies

HeLa cells were co-transfected with SpCas9-WT and RNA pairs; sg35 (INT 4) with either g39 (rs10414837), g58 (rs3761005) or g62 (rs1683564) for strategies 1a, 1b and 2, respectively. 72 h post-transfection, gDNA was extracted and excision efficiency was assessed by measuring the decrease in copy number of exon 1 (strategy 1) or exon 5 (strategy 2), using droplet digital PCR (ddPCR) kits (10042958 and 10031228, Bio-Rad Laboratories). In addition, exon 1 was used to normalize the excision rate of strategy 2 while exon 5 was used to normalize the excision rate of strategy 1. The results disclosed in Table 3 represent the mean % excision±SD (standard deviation) of two independent experiments.

TABLE 3
Tested excision rate for each strategy

| Strategy | Guide-RNA pair | Excision rate (%) |
|---|---|---|
| l.a | g39 + g35 | 49 ± 2 |
| l.b | g58 + g35 | 45 ± 9.6 |
| 2 | g62 + g35 | 41 ± 12.6 |

### Example 3: Assessing Allele Discriminating Editing with Different sgRNAs

Ribonucleoprotein complexes (RNPs) were assembled from the relevant gRNAs targeting the reference sequence and WT-Cas9 (#1081058) or HiFi Cas9 (#1081060) purchased from Integrated DNA Technology (IDT) according to the manufacturer instructions. The RNPs were then nucleofected into iPSCs harboring the relevant SNPs using the 4D-Nucleofecor® System (Lonza). 72 h later, gDNA was extracted, the SNPs regions were amplified, NGS analysis was performed. Allele discrimination was assessed according to % of editing detected in the reference and the alternative alleles. The indel-frequency in each site was calculated using Cas-Analyzer software. Results are summarized in Table 4.

TABLE 4
% Editing using indicated guide sequences

| | % of Editing | | | | | |
|---|---|---|---|---|---|---|
| | rs10414837 (g39) | | rs3761005 (g58) | | rs1683564 (g62) | |
| SpCas9 | Reference | Alternative | Reference | Alternative | Reference | Alternative |
| Varaint | Allele | Allele | Allele | Allele | Allele | Allele |
| WT-Cas9 | 65.3 | 34.7 | 50 | 50 | 100 | 0 |
| HiFi-Cas9 | 94.3 | 5.7 | 49 | 51 | 100 | 0 |

### Example 4: Editing Efficiency in HSCs

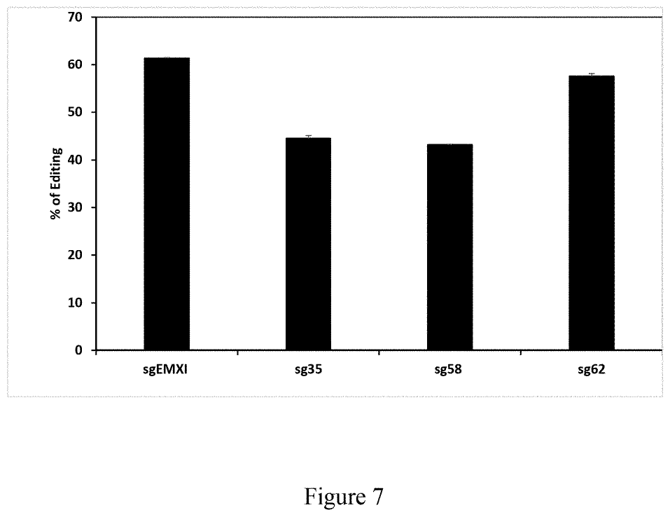

HSCs from healthy donors were nucleofected with RNA components of SpCas9-WT and gRNAs targeting either EMX1 (sgEMX1) or ELANE (g35: INT 4; g58: rs3761005; g62: rs1683564). 72 h post nucleofection gDNA was extracted and editing levels were assayed by indel detection by amplicon analysis (IDAA). FIG. 7 represents the mean % of editing±SD of two independent experiments performed in duplicates.

### Example 5: Functional Maturation Assay

To prove the rescue of the phenotype in corrected cells, a maturation assay starting from patient-derived induced pluripotent cells (iPSC), is prepared from reprogrammed somatic cells. The cells provide a renewable source of patient-derived cells and are shown to accurately replicate the disease phenotype. Briefly, patient PBMCs are transfected with episomal constructs expressing the reprogramming genes, Oct4, Sox2, Nanog, Lin28, L-Myc, Klf4, and SV40LT. Patient-derived iPSCs and normal iPSCs harboring the same SNPs genotype are differentiated into hematopoietic progenitor cells using a commercial kit (STEMdiff™ Hematopoietic Kit, STEMCELL Technologies). After 12 days the differentiation, efficiency is estimated by analyzing the cells for their expression of progenitor markers CD34 and CD45, by flow cytometry analysis. Normal and SCN differentiated progenitor cells are subjected to gene editing and are grown for five (5) days in conditioned media containing stem cell factor (SCF), IL-3, and GM-CSF that promotes the differentiation into neutrophils. SCN edited cells differentiate into neutrophils while unedited SCN-derived cells arrest at earlier differentiation stages. The efficiency of differentiation into neutrophils is measured by detecting neutrophil surface markers (e.g. upregulation of CD16, CD66b and the pan myeloid marker CD33) by flow cytometry.

### Example 6: In Vivo Pilot Dose-Range Finding and Biodistribution Study in Immunodeficient Mice

The dosing schedule, dose range, and route of administration, are studied to determine the presence and number of HSCs with self-renewal and multilineage capacity in immunodeficient NSG mice, following G-CSF administration. A repopulation assay is conducted to determine the presence and number of HSCs that are able to regenerate a functional immune system to establish long term engraftment. For such verification, the NSG strain is used, which is highly supportive of human engraftment and hematopoietic repopulation. The NSG mouse is a NOD SCID mouse lacking mature T cells, B cells, and natural killer (NK) cells, in addition to being deficient in multiple cytokine signaling pathways and having many defects in innate immunity. Engraftment is assessed 16 weeks after primary transplantation (analysis after 12 weeks post-transplant).

The 16-week pilot biodistribution study in NSG mice investigates dose and maximum duration and is conducted at several cell dose levels. The study includes three dose levels plus a group of mice receiving unedited SCN cells. Duration of the pilot study is 16 weeks; however, one of two high dose groups continues for up to 6 months. Mice survive for 6 months in the pilot study, and the duration of the pivotal biodistribution study is 6 months. During the study, persistence of expression via qPCR and immune histochemistry is studied.

### Example 7: Pivotal Biodistribution Study in Immunodeficient Mice

The pivotal biodistribution study utilizes NSG mice and follows the pilot dose-range findings of Example 6. This study is conducted in compliance with Good Laboratory Practice (GLP) and is a pivotal nonclinical pharmacokinetics, pharmacodynamics, and toxicology study. Assessment of toxicity is based on mortality, clinical observations, body and organ weights, and clinical and anatomic pathology following HSC infusion.

Transplantation of gene-edited CD34+ cells into NSG mice requires conditioning in order to provide depletion of endogenous bone marrow and to allow the engraftment of donor cells. Accordingly, busulfan-conditioning is employed to mimic the clinical situation. Three groups (gene edited cells, unedited cells and busulfan vehicle only controls) of 10 male and female mice per group are utilized.

Although the NSG mouse model is not completely similar to the human neutrophil depleted situation, this does not affect the validity of the model for use in the biodistribution study of the gene-edited CD34+ cells, since the mice at any rate are treated with busulfan, depleting the bone marrow function, before the in vivo injection of ex vivo gene edited cells.

An available neutrophil-depleted mouse strain, myeloid cell leukemia 1 (Mcl-1) antiapoptotic protein in Lyz2^{Cre/Cre}Mcl-1^{flox/flox} (Mcl-1^{ΔMyelo}) shows that myeloid-specific deletion of Mcl-1 lead to very severe neutropenia (Csepregi et al. 2018). Mcl-1^{ΔMyelo} mice are able to breed and their survival is close to normal both under specific pathogen-free and conventional housing conditions. However, in contrast to the NSG mouse, there is limited experience with the Mcl-1^{ΔMyelo} mouse model.

### Example 8: An Editing Analysis

Guide sequences comprising 21-30 contiguous nucleotides containing nucleotides in the sequence set forth in SEQ ID NOs: 1-3751 are screened for high on target activity. On target activity is determined by DNA capillary electrophoresis analysis.

According to DNA capillary electrophoresis analysis, guide sequences comprising 21-30 contiguous nucleotides containing nucleotides in the sequence set forth in SEQ ID NOs: 1-3751 are found to be suitable for correction of the ELANE gene.

### Example 9: Efficacy of Allele Specific Knockout of ELANE

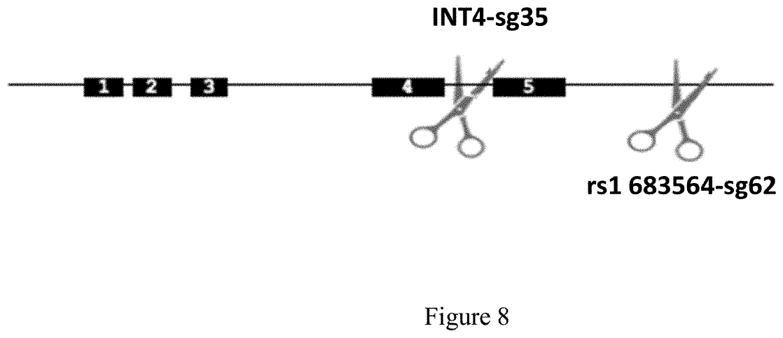

Specific knock-out of the mutated allele of the ELANE gene is mediated by excising intron 4 and exon 5 of the mutant allele of the ELANE gene. This is achieved by mediating a DSB in intron 4 and utilizing SNP rs1683564 for mediating an allele specific DSB as described in FIG. 8. To demonstrate that this strategy effectively enables HSCs to differentiate into mature and functional neutrophils, healthy donors are nucleofected with HSCs (Lonza) with RNPs containing g35 and g62 targeting the respective SNP of intron 4 (See Table 1). Unedited cells are used as a positive control. Forty-eight (48) hours following nucleofection, HSCs are differentiated towards neutrophils according to a published protocol (Zhenwang Jie, et al. Plos One 2017). The differentiation efficiency of the edited and unedited cells is measured by FACS following staining with the neutrophil specific markers CD66b and CD177. To assess the function of the HSC-mediated neutrophils, the following assays are performed:
1. Phagocytosis is assayed using the EZCell™ Phagocytosis Assay Kit (BioVision). The kit utilizes pre-labeled Zymosan particles as a tool for rapid and accurate detection and quantification of in vitro phagocytosis by flow cytometry.
2. A killing assay is conducted by incubating the HSCs-derived neutrophils with E. coli, with the bacteria then seeded on agar plate for colonies formation. Untreated bacteria or bacteria that were incubated with undifferentiated HSCs are used as controls. The killing efficiency is calculated as follows: (# of colonies_{Neutrophils}/# of colonies_{Control})×100.
3. Chemotaxis is assayed using the EZCell™ Cell Migration/Chemotaxis Assay Kit (Biovision).

### Example 10: Subject Selection for Treatment

Step 1: Four patients A-D diagnosed with SCN or CyN are screened by Exon sequencing to identify an ELANE pathogenic mutation in the ELANE gene. Step 2: Subjects with an identified mutation are then screened by Sanger sequencing to confirm heterozygosity of at least one of rs1683564, rs10414837 and rs3761005. Step 3: For each subject determined to be heterozygous at least one of rs1683564, rs10414837 and rs3761005, the nucleotide of the heterozygous SNP on the mutant allele of the ELANE gene is determined using BAC bio. Step 4: Appropriate guides are selected according to Table 5. The length of the selected guide is determined in accordance to the nuclease that is used to target the heterozygous SNP. In a non-limiting example, SpCas9 is used in conjunction with an RNA molecule having a guide sequence portion of 20 nucleotides. In a non-limiting example, OMNI-50 nuclease is used in conjunction with an RNA molecule having a guide sequence portion of 22 nucleotides.

TABLE 5
Guides Designed for Discriminating SNPs and used for the Editing Strategy

| Target | | SEQ | Guide | | |
|---|---|---|---|---|---|
| SNP | gRNA | ID NO: | Sequence | Location | Mechanism |
| rs10414837 | g39 | 3783 | CAGCGGGUG | Promoter | Excision, |
| | REF 20 nt | | UAGACUCCG | region | allele knock |
| | | | AG | | out |
| | g39 ALT 20 | 3784 | CAGCAGGUG | | |
| | nt | | UAGACUCCG | | |
| | | | AG | | |
| | g39 | 254 | CACAGCGGG | | |
| | REF 22nt | | UGUAGACUC | | |
| | sgRNA- | | CGAG | | |
| | 837US1-ref | | | | |
| | g39 ALT | 341 | CACAGCAGG | | |
| | 22nt | | UGUAGACUC | | |
| | sgRNA- | | CGAG | | |
| | 837US1-alt | | | | |
| rs3761005 | g58 | 3785 | GCUGCGGGA | Promoter | Excision, |
| | REF 20 nt | | AAGGGAUUC | region | allele knock |
| | | | CC | | out |
| | g58 | 3786 | GCUGCGGGA | | |
| | ALT 20 nt | | AUGGGAUUC | | |
| | | | CC | | |
| | sgRNA- | 1139 | CAGCUGCGG | | |
| | 005US2-ref | | GAAAGGGAU | | |
| | 22 nt | | UCCC | | |
| | sgRNA- | 1228 | CAGCUGCGG | | |
| | 005US2-alt | | GAAUGGGAU | | |
| | 22 nt | | UCCC | | |
| rs1683564 | 862 | 3787 | GUCAAGCCCC | Downstream | Excision, |
| | REF 20 nt | | AGAGGCCAC | to 3’UTR | allele knock |
| | | | A | | out |
| | ALT 20 nt | 3788 | GUCAAGCCCC | | |
| | | | AGAGGACAC | | |
| | | | A | | |
| | sgRNA- | 2517 | GUGUCAAGC | | |
| | 564DS-ref 22 | | CCCAGAGGC | | |
| | nt | | CACA | | |
| | sgRNA- | 2601 | GUGUCAAGC | | |
| | 564DS-alt 22 | | CCCAGAGGA | | |
| | nt | | CACA | | |
| | g35 20 nt | 2992 | AGUCCGGGC | Intron 4 | Excision, |
| | | | UGGGAGCGG | | allele knock |
| | | | GU | | out |
| | sgRNA- | 3633 | GCAGUCCGG | | |
| | Constant 22 | | GCUGGGAGC | | |
| | nt | | GGGU | | |

Step 5: The guides selected are introduced to HSCs obtained from each respective subject and reduction in the pathogenic ELANE mutation in the HSCs is verified by Next Generation Sequencing. The methodology for patients A-D is illustrated below:
1. Patient A is screened according to step 1 and found to have a known pathogenic mutation in his ELANE gene, in agreement with his phenotype and clinical condition. Patient A is screened according to step 2, around the SNPs of interest, and is found to be homozygous for all three SNP—rs10414837, rs1683564 and rs3761005. Patient A is determined to not eligible for treatment.
2. Patient B is verified for a known pathogenic ELANE mutation. Patient B is screened according to step 2 and is found to be homozygous for SNPs rs1683564 and rs10414837. Patient B is found to be heterozygous for rs3761005 in the promotor region. Patient B is determined to be eligible for treatment. According to step 3, the nucleotide of the SNP residing on the same allele as the pathogenic ELANE mutation (linkage determination) is determined. Patent B is found to have the reference nucleotide base in the rs3761005 SNP position on the same allele as the ELANE pathogenic mutation. g58ref is fully complementary to this SNP presentation and is selected in combination with g35 directed at the non-coding region of intron 4. According to step 4, the chosen guide composition includes a pair of guides g58ref and g35. Successful excision of the mutated allele using the selected pair of guides is verified according to step 5 on the patient HSCs using NGS readout.
3. Patient C suffers from Severe Neutropenia since early childhood. Screening according to step A, no pathogenic mutation is found in his ELANE gene. Patient C is determined to not eligible for treatment.
4. Patient D is verified to have a known pathogenic mutation in ELANE gene, and when genotyping his SNPs according to step 2, he is found to be heterozygous at 2 out of 3 SNPs—both rs10414837 and rs1683564 are found to be heterozygous while rs3761005 is found to be homozygous. A selection between two possible SNPs to use for the gene manipulation is made. In order to make the selection, step 3 is performed to determine the linkage between the SNPs and the pathogenic mutation, i.e. determination of which nucleotide of the SNP (reference or alternative) resides on the same allele as the ELANE pathogenic mutation (SNP presentation). Patient D is determined to have the reference presentation of rs10414837 and sg39ref is determined to be appropriate for use. Refering to the rs1683564 SNP, the alternative presentation is found to be linked to the ELANE pathogenic mutation, sg62alt is determined to be the appropriate guide for use. Each of these guides is used in combination with g35. According to Step 4, two pairs of possible guides compositions are identifed: sg39ref+g35, and g62alt+g35. To determine which of the guide pairs is preferable, a database of editing properties and characterization of each of the guides and guide pairs is assessed to determine off-target and editing efficiencies A guide pair is selected based on the database assessment, and is utilized according to step 5 on HSCs providing an NGS readout.

### Example 11: Nucleases with Improved Activity with 21 and 22 Nucleotide Guides

CRISPR repeat (crRNA), transactivating crRNA (tracrRNA), nucleases polypeptide and PAM sequences were predicted from different metagenomic databases of sequences of environmental samples. The list of bacterial species/strains from which the CRISPR repeat, tracRNA sequence and nucleases polypeptide sequence were predicted is provided in Table 6.

TABLE 6
DNA and Protein Sequences of Nucleases

| | | | DNA | DNA |
|---|---|---|---|---|
| | | Protein | Sequence | Sequence |
| | | Sequence | SEQ ID NO | SEQ ID NO |
| | | SEQ ID | (Original | (human |
| Name | Source Organism | NO: | ORF) | optimized) |
| OMNI-50 | Ezakiella peruensis | 3789 | 3790 | 3791 |
| | strain M6.X2 | | | |

### Construction of OMNI-Nuclease Polypeptides.

The open reading frame of potential OMNI candidates that were identified were codon optimized for E. coli and for human cell line expression. The E. coli optimized ORF was cloned into bacterial plasmid, pb-NNC and the human optimized into pmOMNI plasmid (Table 7).

TABLE 7
Plasmids and constructs

| | | | | SEQ ID |
|---|---|---|---|---|
| | | | | NO of |
| | | | | DNA |
| Plasmid | Purpose | Elements | Example | sequence |
| pET9a | Expression | T7 promoter—SV40 | pET9a | 3792 |
| | and | NLS—OMNI ORF | OMNI-50- | |
| | purification | (human optimized)— | HisTag | |
| | of OMNI | HA—SV40 NLS—8 | | |
| | proteins | His-tag—T7 terminator | | |
| pmOMNI | Expressing | CMV promoter— | pmOMNI | 3793 |
| | OMNI | Kozak—SV40 NLS— | OMNI50 | |
| | polypeptide | OMNI ORF (human | | |
| | in the | optimized)—HA— | | |
| | mammalian | SV40 NLS—P2A— | | |
| | system | mCheny—bGH poly(A) | | |
| | | signal | | |
| pmGuide | Expressing | U6 promoter— | pmGuide | 3794 |
| Endogenic | OMNI | Endogenicnspacer | OMNI50 | |
| site | sgRNA in | sgRNA scaffold | ELANE | |
| | the | | g35 | |
| | mammalian | | sgRNA | |
| | system | | V2 | |

TABLE 7 APPENDIX
Details of construct elements

| Element | Protein Sequence | DNA sequence |
|---|---|---|
| HA Tag | SEQ ID NO: 3795 | SEQ ID NO: 3796 |
| NLS | SEQ ID NO: 3797 | SEQ ID NO: 3798 |
| P2A | SEQ ID NO: 3799 | SEQ ID NO: 3800 |
| mCherry | SEQ ID NO: 3801 | SEQ ID NO: 3802 |

### Expression of OMNI Nucleases Coded by an Optimized DNA Sequence in Mammalian Cells

First, expression of the optimized DNA sequences coding for the OMNI-50 protein in mammalian cells, was validated. To this end, an expression vector coding for an HA-tagged OMNI nuclease or Streptococcus pyogenes Cas9 (SpCas9) linked by P2A peptide to mCherry (pmOMNI, Table 7) was introduced into HeLa cells using the Jet-optimus™ transfection reagent (polyplus-transfection). P2A peptide is a self-cleaving peptide which can induce the cleavage of the recombinant protein in cell, such that the OMNI nuclease and the mCherry are separated upon expression and the mCherry can serve as indicator for transcription efficiency of the OMNI from the expression vector. The level of transcription for OMNI-50 in the activity assays was determined using flow cytometry.

### Activity in Human Cells on Endogenous Genomic Targets

OMNI-50 was also assayed for its ability to promote editing on specific genomic locations in human cells. To this end, OMNI-50 was transfected into HeLa cells together with sgRNA designed to target specific locations in the human genome (pmGuide, Table 7). At 72 h, cells were harvested, and half were used for quantification of transfection efficiency by FACS using mCherry fluorescence as marker. The rest of the cells were lysed, and their genomic DNA content was used in PCR reaction, amplifying the corresponding putative genomic targets. Amplicons were subjected to NGS and the resulting sequences were then used calculate the percentage of editing events in each target site. Short Insertions or deletions (indels) around the cut site are the typical outcome of repair of DNA ends following nuclease-induced DNA cleavage. The calculation of % editing was therefore deduced from the fraction of indels containing sequences within each amplicon. All editing values were normalized to the transfection and translation efficacy obtained for each experiment and deduced from the percentage of mCherry expressing cells. The normalized values represent the effective editing levels within the population of cells that expressed the nuclease. Genomic activity of OMNI-50 was assessed using a panel of 11 unique sgRNAs, each designed to target a different genomic location. The results of these experiments are summarized in Table 8. As can be seen in the table, OMNI-50 exhibits high and significant editing levels compared to the negative control in all target sites tested.

TABLE 8
Activity in human cells on endogenous genomic targets

| | | | | 3′ | | | | | | |
|---|---|---|---|---|---|---|---|---|---|---|
| | | | | (PAM | | | | | % | Norm, |
| | | | | con- | | | | | trans- | % |
| | | Corre- | | taining | | | | % | fection | editing |
| | | sponding | | genomic | | % | Norm, | editing | in | in |
| Nu- | Genomic | Spacer | Spacer | seq | % | trans- | % | in neg | neg | neg |
| clease | site | name | sequence | Bold | indels | fection | editing | control | control | control |
| OMNI- | EMXI | EMX1g1_ | UCUGUGA | GGGA | 44.18 | | | 0.02 | | |
| 50 | site 2 | OMNI- | AUGUUAG | GCAG | -25.72 | | | | | |
| | | 50 | ACCCAU | | | | | | | |
| | | | (SEQ ID | | | | | | | |
| | | | NO: 3803) | | | | | | | |
| | EMXI | EMX1g2_ | CCAUGGG | AGGG | 55.81 | | | | | |
| | site 3 | OMNI- | AGCAGCU | GACC | | | | | | |
| | | 50 | GGUCAG | | | | | | | |
| | | | (SEQ ID | | | | | | | |
| | | | NO: 3804) | | | | | | | |
| | CXCR4 | CXCR4g1_ | GCAAGAG | AGGA | 29.58 | | | 0.18 | | |
| | site 3 | OMNI- | ACCCACA | GCGC | -32.14 | | | | | |
| | | 50 | CACCGG | | | | | | | |
| | | | (SEQ ID | | | | | | | |
| | | | NO: 3805) | | | | | | | |
| | CXCR4 | CXCR4g2_ | ACACCGG | TGGG | 42.13 | | | 0.22 | | |
| | site 4 | OMNI- | AGGAGCG | GGAG | -49.85 | | | | | |
| | | 50 | CCCGCU | | | | | | | |
| | | | (SEQ ID | | | | | | | |
| | | | NO: 3806) | | | | | | | |
| | PDCD1 | PDCD1g1_ | CGUCUGG | TGGG | 13.35 | | | 0.05 | | |
| | site 4 | OMNI- | GCGGUGC | CTGG | -8.7 | | | | | |
| | | 50 | UACAAC | | | | | | | |
| | | | (SEQ ID | | | | | | | |
| | | | NO: 3807) | | | | | | | |
| | PDCD1 | PDCD1g2_ | CUACAAC | AGGA | 17.53 | | | | | |
| | site 5 | OMNI- | UGGGCUG | TGGT | | | | | | |
| | | 50 | GCGGCC | | | | | | | |
| | | | (SEQ ID | | | | | | | |
| | | | NO: 3808) | | | | | | | |
| | ELANE | ELANEg35_ | AGUCCGG | GGGG | 40.92 | | | 0.24 | 5.95 | 3.9822 |
| | g35 | OMNI- | GCUGGGA | AGCA | -55.39 | | | | | 25429 |
| | | 50 | GCGGGU | | | | | | | |
| | | | (SEQ ID | | | | | | | |
| | | | NO: 2992) | | | | | | | |
| | ELANE | ELANEg58_ | GCUGCGG | TGGG | 11.11 | 20.50 | 54.23 | 0.18 | 5.95 | 2.9745 |
| | g58 | OMNI- | GAAAGGG | ACTC | | | | | | 53445 |
| | | 50 | AUUCCC | | | | | | | |
| | | | (SEQ ID | | | | | | | |
| | | | NO: 3809) | | | | | | | |
| | ELANE | ELANEg38_ | ACAGCGG | GGGG | 9.99 | | | | | |
| | g38 | OMNI- | GUGUAGA | GACG | | | | | | |
| | | 50 | CUCCGA | | | | | | | |
| | | | (SEQ ID | | | | | | | |
| | | | NO: 3810) | | | | | | | |
| | ELANE | ELANEg39_ | CAGCGGG | GGGG | 24.87 | | | | | |
| | g39 | OMNI- | UGUAGAC | ACGT | | | | | | |
| | | 50 | UCCGAG | | | | | | | |
| | | | (SEQ ID | | | | | | | |
| | | | NO: 3811) | | | | | | | |
| | ELANE | ELANEg62_ | GUCAAGC | GGGA | 38.87 | | | 0.12 | 5.95 | 2.0025 |
| | 862 | OMNI- | CCCAGAG | CAGA | -52.74 | | | | | 03126 |
| | | 50 | GCCACA | | | | | | | |
| | | | (SEQ ID | | | | | | | |
| | | | NO: 3812) | | | | | | | |

### Intrinsic Fidelity in Human Cells:

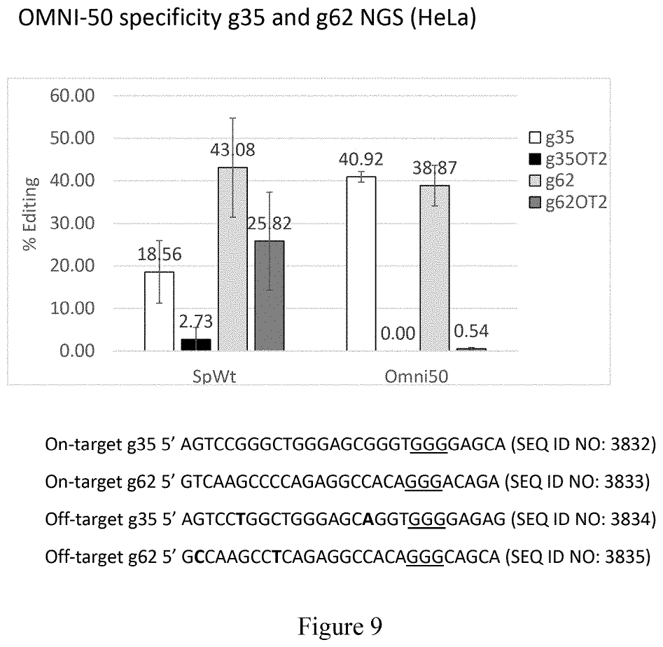

The intrinsic fidelity of a nuclease is a measure of its cleavage specificity. A high fidelity nuclease is a nuclease that promotes cleavage on the intended target (“on-target”) with minimal or no cleavage of the unintended target (“Off-target”). In CRISPR nuclease the target is acquired based on sequence complementarity to the spacer element of the guide RNA. Off targeting results from similarity of an unintended target to the spacer sequence. The intrinsic fidelity of OMNIs at the genomic level in human cells was measured by conducting an activity assay as described, following PCR amplification, NGS, and InDel analysis for both the on-target region and a pre-validated off-target region. A measurement of intrinsic fidelity for OMNI-50 is provided in FIG. 9. In this example, OMNI-50 fidelity was measured using two guide RNAs independently, in each case a side-by-side measurement of SpCas9 is provided for reference. The first site was targeted using the ELANE g35 gRNA (Table 8) which has a defined on-target upstream to the ELANE gene on chr19 and off-target on chr15. As can be seen in FIG. 9, the on/off target editing efficiency ratio obtained by OMNI-50 was 41:0 while the SpCas9 on/off ratio is 6.8:1 (40.9%/0%; 18.6%/2.7% respectively). The second site was targeted by ELANE g62 gRNA (Table 8). This gRNA spacer sequence has a defined on-target at the ELANE gene on chr19 and an off-target on chr1. In this case, the On/Off ratio obtained by OMNI-50 was 72:1 compare to 1.7:1 ratio obtained by SpCas9 (38.9%/0.6%; 43.1%/25.8% respectively). These results demonstrate that OMNI-50 has a significantly higher intrinsic fidelity in comparison to SpCas9 in a human cellular environment.

### Purification of OMNIs Protein.

OMNI-50 open-reading frame was cloned into bacterial expression plasmids (T7-NLS-OMNI-NLS-HA-His-tag, pET9a, Table 7) and expressed in C43 cells (Lucigen). Cells were grown in Terrific Broth to mid-log phase and the temperature lowered to 18° C. Expression was induced at 0.6 OD with 1 mM IPTG for 16-20 h before harvesting and freezing cells at −80° C. Cell paste was resuspended in lysis buffer (50 mM NaH₂PO₄, 300 mM NaCl, 10 mM imidazole pH8.0, 1 mM TCEP) supplemented with EDTA-free complete protease inhibitor cocktail set III (Calbiochem). Cells were lysed using sonication and cleared lysate was incubated with Ni-NTA resin. The resin was loaded onto gravity column and washed with wash buffer (50 mM NaH₂PO₄, 300 mM NaCl, 50 mM imidazole pH8.0, 1 mM TCEP) and OMNI protein was eluted with wash buffer supplemented with 100-500 mM Imidazole. Fractions containing OMNI protein were pooled and concentrated and loaded onto a centricone (Amicon Ultra 15 ml 100K, Merck), and buffer exchanged to GF buffer (50 mM Tris-HCl pH 7.5, 500 mM NaCl, 10% glycerol, 0.4M Arginine). The concentrated OMNI protein was further purified by SEC on HiLoad 16/600 Superdex 200 pg-SEC, AKTA Pure (GE Healthcare Life Sciences) with a 50 mM Tris-HCl pH 7.5, 500 mM NaCl, 10% glycerol, 0.4M Arginine. Fractions containing OMNI protein were pooled, concentrated, and loaded onto a centricone (Amicon Ultra 15 ml 100K, Merck) with a final storage buffer of 10 mM Tris-HCl, pH 7.5, 150 mM NaCl, 10% glycerol and 1 mM TCEP. Purified OMNI protein was concentrated to 10 mg/ml stocks and flash-frozen in liquid nitrogen and stored at −80° C.

### Guide Optimization by RNP Activity Assay.

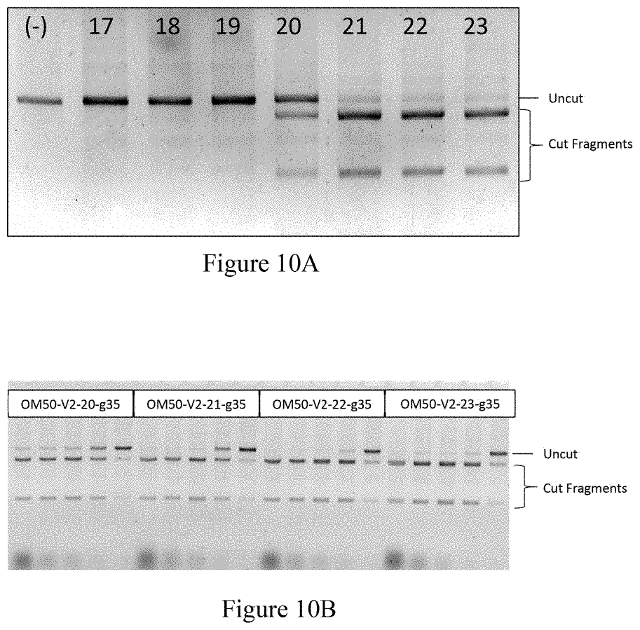

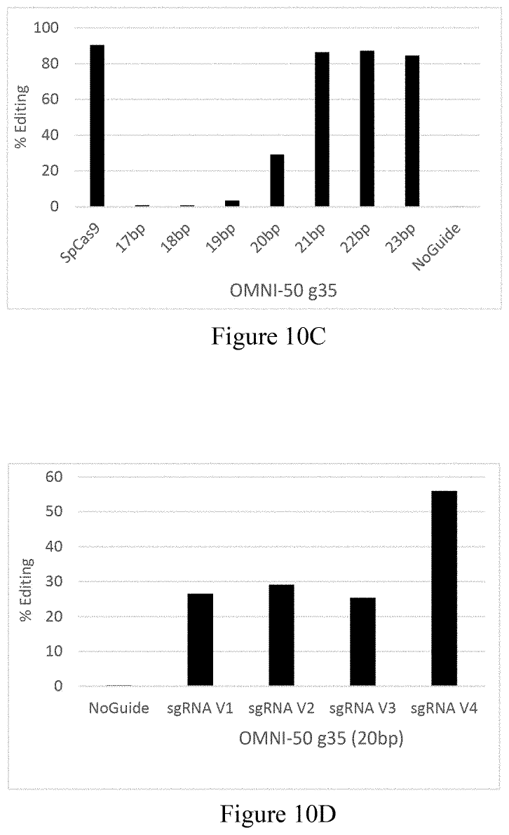

Synthetic sgRNAs of OMNI-50 were synthesized with three 2′-O-methyl 3′-phosphorothioate at the 3′ and 5′ ends (Agilent). Activity assay of OMNI-50 RNP with different spacer lengths (17-23 nucleotides) of guide 35 (Table 9, FIG. 10A). Briefly, 4 pmol of OMNI-50 nuclease were mixed with 6 pmol of synthetic guide. After 10 minutes of incubation at room-temp, the RNP complexes were reacted with 100 ng template of On-target. Only spacers larger than 22 nucleotides show near full cleavage of the On-target template. Furthermore, reducing amounts of RNPs (4, 2, 1.2, 0.6 and 0.2 μmol) with spacer lengths 20-23 were reacted with 100 ng DNA target template (FIG. 10B). Spacer at lengths 22 nucleotides shows better cleavage activity even at lower RNP concentrations.

TABLE 9
synthetic sgRNA (RNP)

| | O50_ | O50_ | O50_ | O50_ | O50_ |
|---|---|---|---|---|---|
| | ELANE_V2_ | ELANE_V2_ | ELANE_V2_ | ELANE_V2_ | ELANE_V2_ |
| Name | g35_23 | g35_22 | g35_21 | g35_20 | g35_19 |
| Spacer | UgcAGUCC | gcAGUCCG | cAGUCCGG | AGUCCGG | GUCCGGG |
| | GGGCUGG | GGCUGGG | GCUGGGA | GCUGGGA | CUGGGAG |
| | GAGCGGG | AGCGGGU | GCGGGU | GCGGGU | CGGGU |
| | U (SEQ ID | (SEQ ID NO: | (SEQ ID NO: | (SEQ ID NO: | (SEQ ID NO: |
| | NO: 3813) | 3633) | 3273) | 2992 | 3814) |
| Scaffold | gUUUGAG | gUUUGAG | gUUUGAG | gUUUGAG | gUUUGAG |
| | AGUUAUG | AGUUAUG | AGUUAUG | AGUUAUG | AGUUAUG |
| | UAAgaaaU | UAAgaaaU | UAAgaaaU | UAAgaaaU | UAAgaaaU |
| | UACAUGA | UACAUGA | UACAUGA | UACAUGA | UACAUGA |
| | CGAGUUC | CGAGUUC | CGAGUUC | CGAGUUC | CGAGUUC |
| | AAAUAAA | AAAUAAA | AAAUAAA | AAAUAAA | AAAUAAA |
| | AAUUUAU | AAUUUAU | AAUUUAU | AAUUUAU | AAUUUAU |
| | UCAAACC | UCAAACC | UCAAACC | UCAAACC | UCAAACC |
| | GCCUAUU | GCCUAUU | GCCUAUU | GCCUAUU | GCCUAUU |
| | UAUAGGC | UAUAGGC | UAUAGGC | UAUAGGC | UAUAGGC |
| | CGCAGAU | CGCAGAU | CGCAGAU | CGCAGAU | CGCAGAU |
| | GUUCUGC | GUUCUGC | GUUCUGC | GUUCUGC | GUUCUGC |
| | AUUAUGC | AUUAUGC | AUUAUGC | AUUAUGC | AUUAUGC |
| | UUGCUAU | UUGCUAU | UUGCUAU | UUGCUAU | UUGCUAU |
| | UGCAAGC | UGCAAGC | UGCAAGC | UGCAAGC | UGCAAGC |
| | UUUUUU | UUUUUU | UUUUUU | UUUUUU | UUUUUU |
| | (SEQ ID NO: | (SEQ ID NO: | (SEQ ID NO: | (SEQ ID NO: | (SEQ ID NO: |
| | 3815) | 3815) | 3815) | 3815) | 3815) |
| Version | V2 | V2 | V2 | V2 | V2 |
| Full sgRNA | UgcAGUCC | gcAGUCCG | cAGUCCGG | AGUCCGG | GUCCGGG |
| sequence | GGGCUGG | GGCUGGG | GCUGGGA | GCUGGGA | CUGGGAG |
| | GAGCGGG | AGCGGGU | GCGGGUg | GCGGGUg | CGGGUgU |
| | UgUUUGA | gUUUGAG | UUUGAGA | UUUGAGA | UUGAGAG |
| | GAGUUAU | AGUUAUG | GUUAUGU | GUUAUGU | UUAUGUA |
| | GUAAgaaa | UAAgaaaU | AAgaaaUU | AAgaaaUU | AgaaaUUAC |
| | UUACAUG | UACAUGA | ACAUGAC | ACAUGAC | AUGACGA |
| | ACGAGUU | CGAGUUC | GAGUUCA | GAGUUCA | GUUCAAA |
| | CAAAUAA | AAAUAAA | AAUAAAA | AAUAAAA | UAAAAAU |
| | AAAUUUA | AAUUUAU | AUUUAUU | AUUUAUU | UUAUUCA |
| | UUCAAAC | UCAAACC | CAAACCG | CAAACCG | AACCGCC |
| | CGCCUAU | GCCUAUU | CCUAUUU | CCUAUUU | UAUUUAU |
| | UUAUAGG | UAUAGGC | AUAGGCC | AUAGGCC | AGGCCGC |
| | CCGCAGA | CGCAGAU | GCAGAUG | GCAGAUG | AGAUGUU |
| | UGUUCUG | GUUCUGC | UUCUGCA | UUCUGCA | CUGCAUU |
| | CAUUAUG | AUUAUGC | UUAUGCU | UUAUGCU | AUGCUUG |
| | CUUGCUA | UUGCUAU | UGCUAUU | UGCUAUU | CUAUUGC |
| | UUGCAAG | UGCAAGC | GCAAGCU | GCAAGCU | AAGCUUU |
| | CUUUUUU | UUUUUU(S | UUUUU | UUUUU | UUU (SEQ |
| | (SEQ ID NO: | EQ ID NO: | (SEQ ID NO: | (SEQ ID NO: | ID NO: |
| | 3816) | 3817) | 3818) | 3819) | 3820) |
| Protospacer | CTGTTGCT | CTGTTGCT | CTGTTGCT | CTGTTGCT | CTGTTGCT |
| (with PAM = | GCAGTCC | GCAGTCC | GCAGTCC | GCAGTCC | GCAGTCC |
| Bold)-On | GGGCTGG | GGGCTGG | GGGCTGG | GGGCTGG | GGGCTGG |
| target | GAGCGGG | GAGCGGG | GAGCGGG | GAGCGGG | GAGCGGG |
| | TGGGGAG | TGGGGAG | TGGGGAG | TGGGGAG | TGGGGAG |
| | CAGAGGG | CAGAGGG | CAGAGGG | CAGAGGG | CAGAGGG |
| | (SEQ ID NO: | (SEQ ID NO: | (SEQ ID NO: | (SEQ ID NO: | (SEQ ID NO: |
| | 3821) | 3821) | 3821) | 3821) | 3821) |
| Protospacer | GTTAAGAg | GTTAAGAg | GTTAAGAg | GTTAAGAg | GTTAAGAg |
| (with PAM = | aCAGTCCa | aCAGTCCa | aCAGTCCa | aCAGTCCa | aCAGTCCa |
| Bold)- | GGCTGGG | GGCTGGG | GGCTGGG | GGCTGGG | GGCTGGG |
| Off target | AGCaGGT | AGCaGGT | AGCaGGT | AGCaGGT | AGCaGGT |
| | GGGGAGA | GGGGAGA | GGGGAGA | GGGGAGA | GGGGAGA |
| | GGAGGG | GGAGGG | GGAGGG | GGAGGG | GGAGGG |
| | (SEQ ID NO: | (SEQ ID NO: | (SEQ ID NO: | (SEQ ID NO: | (SEQ ID NO: |
| | 3822) | 3822) | 3822) | 3822) | 3822) |

| | O50_ | O50_ | O50_ | O50_ |
|---|---|---|---|---|
| | ELANE_V2_ | ELANE_V2_ | ELANE_V3_ | ELANE_V4_ |
| Name | g35_18 | g35_17 | g35_20 | g35_20 |
| Spacer | UCCGGGCUG | CCGGGCUGG | AGUCCGGGC | AGUCCGGGC |
| | GGAGCGGGU | GAGCGGGU | UGGGAGCGG | UGGGAGCGG |
| | (SEQ ID NO: | (SEQ ID NO: | GU (SEQ ID | GU (SEQ ID |
| | 3823) | 3824) | NO: 2992) | NO: 2992) |
| Scaffold | gUUUGAGAG | gUUUGAGAG | gUUUGAGAG | gUUUGAGAG |
| | UUAUGUAAga | UUAUGUAAga | UUAUGUgaaaA | UUAUGUAgaaa |
| | aaUUACAUGA | aaUUACAUGA | CAUGACGAG | UACAUGACG |
| | CGAGUUCAA | CGAGUUCAA | UUCAAAUAA | AGUUCAAAU |
| | AUAAAAAUU | AUAAAAAUU | AAAUUUAUU | AAAAAUUUA |
| | UAUUCAAAC | UAUUCAAAC | CAAACCGCCU | UUCAAACCG |
| | CGCCUAUUU | CGCCUAUUU | AUUUAUAGG | CCUAUUUAU |
| | AUAGGCCGC | AUAGGCCGC | CCGCAGAUG | AGGCCGCAG |
| | AGAUGUUCU | AGAUGUUCU | UUCUGCAUU | AUGUUCUGC |
| | GCAUUAUGC | GCAUUAUGC | AUGCUUGCU | AUUAUGCUU |
| | UUGCUAUUG | UUGCUAUUG | AUUGCAAGC | GCUAUUGCA |
| | CAAGCUUUU | CAAGCUUUU | UUUUUU (SEQ | AGCUUUUUU |
| | UU (SEQ ID | UU (SEQ ID | ID NO: 3825) | (SEQ ID NO: |
| | NO: 3815) | NO: 3815) | | 3826) |
| Version | V2 | V2 | V3 | V4 |
| Full sgRNA | UCCGGGCUG | CCGGGCUGG | AGUCCGGGC | AGUCCGGGC |
| sequence | GGAGCGGGUg | GAGCGGGUgU | UGGGAGCGG | UGGGAGCGG |
| | UUUGAGAGU | UUGAGAGUU | GUgUUUGAG | GUgUUUGAG |
| | UAUGUAAgaaa | AUGUAAgaaaU | AGUUAUGUga | AGUUAUGUA |
| | UUACAUGAC | UACAUGACG | aaACAUGACG | gaaaUACAUGA |
| | GAGUUCAAA | AGUUCAAAU | AGUUCAAAU | CGAGUUCAA |
| | UAAAAAUUU | AAAAAUUUA | AAAAAUUUA | AUAAAAAUU |
| | AUUCAAACC | UUCAAACCG | UUCAAACCG | UAUUCAAAC |
| | GCCUAUUUA | CCUAUUUAU | CCUAUUUAU | CGCCUAUUU |
| | UAGGCCGCA | AGGCCGCAG | AGGCCGCAG | AUAGGCCGC |
| | GAUGUUCUG | AUGUUCUGC | AUGUUCUGC | AGAUGUUCU |
| | CAUUAUGCU | AUUAUGCUU | AUUAUGCUU | GCAUUAUGC |
| | UGCUAUUGC | GCUAUUGCA | GCUAUUGCA | UUGCUAUUG |
| | AAGCUUUUU | AGCUUUUUU | AGCUUUUUU | CAAGCUUUU |
| | U (SEQ ID | (SEQ ID NO: | (SEQ ID NO: | UU (SEQ ID |
| | NO: 3827) | 3828) | 3829) | NO: 3830) |
| Protospacer | CTGTTGCTGC | CTGTTGCTGC | CTGTTGCTGC | CTGTTGCTGC |
| (with PAM = | AGTCCGGGCT | AGTCCGGGCT | AGTCCGGGCT | AGTCCGGGCT |
| Bold)-On | GGGAGCGGG | GGGAGCGGG | GGGAGCGGG | GGGAGCGGG |
| target | TGGGGAGCA | TGGGGAGCA | TGGGGAGCA | TGGGGAGCA |
| | GAGGG (SEQ | GAGGG (SEQ | GAGGG (SEQ | GAGGG (SEQ |
| | ID NO: 3821) | ID NO: 3821) | ID NO: 3821) | ID NO: 3821) |
| Protospacer | GTTAAGAgaC | GTTAAGAgaC | GTTAAGAgaC | GTTAAGAgaC |
| (with PAM = | AGTCCaGGCT | AGTCCaGGCT | AGTCCaGGCT | AGTCCaGGCT |
| Bold)- | GGGAGCaGGT | GGGAGCaGGT | GGGAGCaGGT | GGGAGCaGGT |
| Off target | GGGGAGAGG | GGGGAGAGG | GGGGAGAGG | GGGGAGAGG |
| | AGGG (SEQ ID | AGGG (SEQ ID | AGGG (SEQ ID | AGGG (SEQ ID |
| | NO: 3822) | NO: 3822) | NO: 3822) | NO: 3822) |

Spacer length optimization was also performed in a cell line context. RNPs were assembled by mixing 100 uM nuclease with 120 uM of synthetic guide with different spacer lengths (17-23 nt, Table 4) (and 100 uM Cas9 electroporation enhancer (IDT). After 10 min of incubation at room-temp, the RNP complexes were mixed with 200,000 pre-washed U2OS cells and electroporated using Lonza SE Cell Line 4D-Nucleofector X Kit with the DN100 program, according to the manufacture's protocol. At 72 h cells were lysed, and their genomic DNA content was used in PCR reaction, amplifying the corresponding putative genomic targets. Amplicons were subjected to NGS and the resulting sequences were then used calculate the percentage of editing events. As can be seen in FIG. 10C spacers of 17-19 nucleotides show low editing level, 20 nucleotides spacers show medium editing, and spacers of 21-23 nucleotides show the highest editing level.

Using the same cell line context, different versions of tracer RNA modifications were also tested (Table 9). The different versions of sgRNA were tested with a 20 nucleotide spacer. As can be seen in FIG. 10D, RNP assembly using either sgRNA V1, V2, and V3 results in similar editing levels. However, RNP assembly using sgRNA V4 results in a significantly higher editing level.

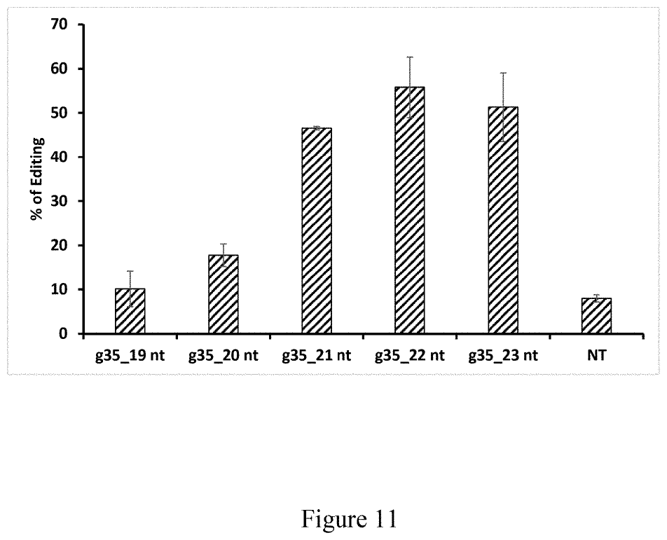

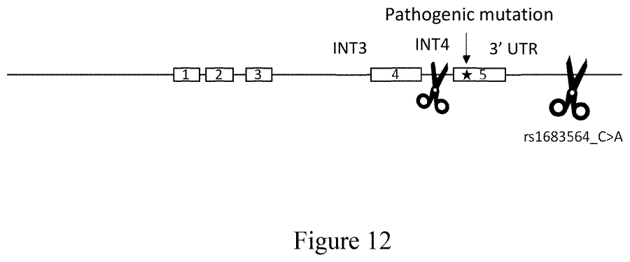

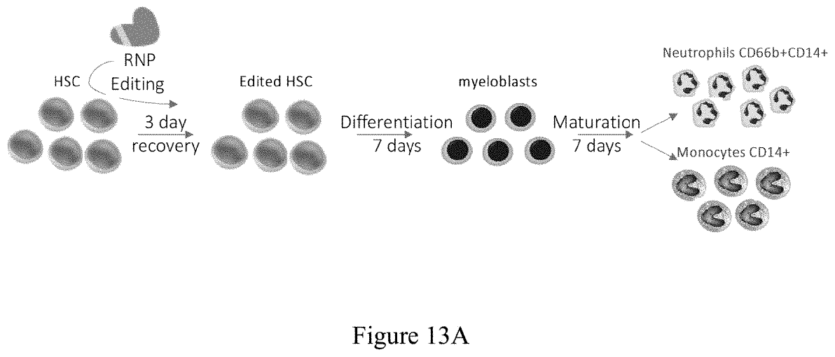

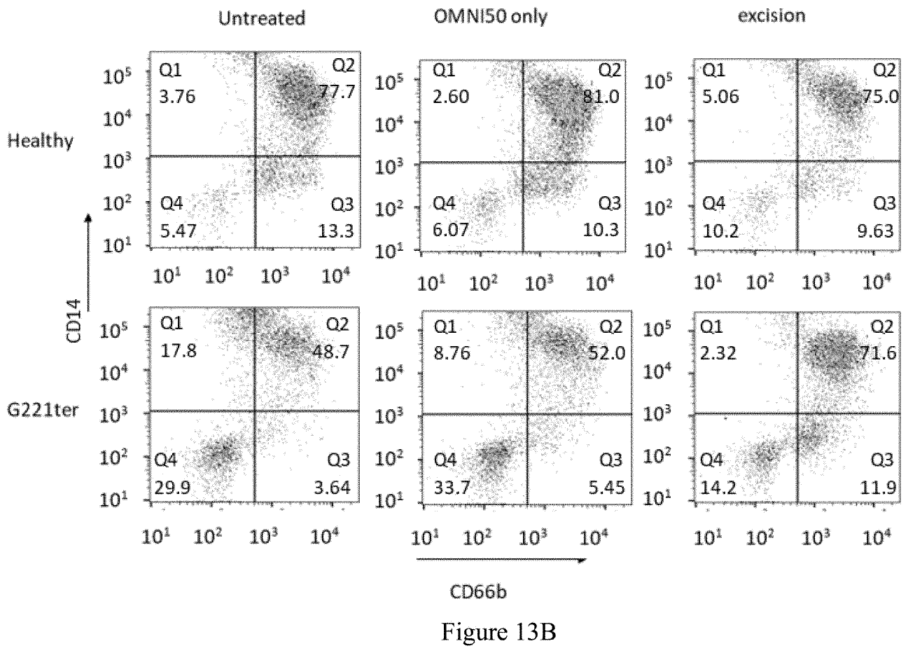

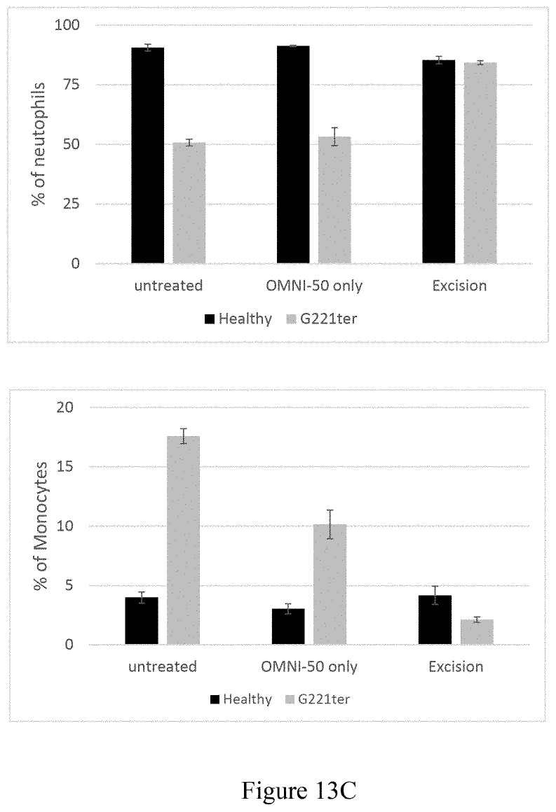

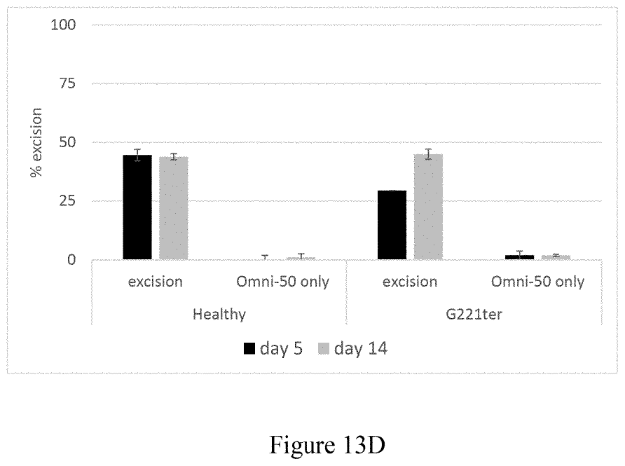

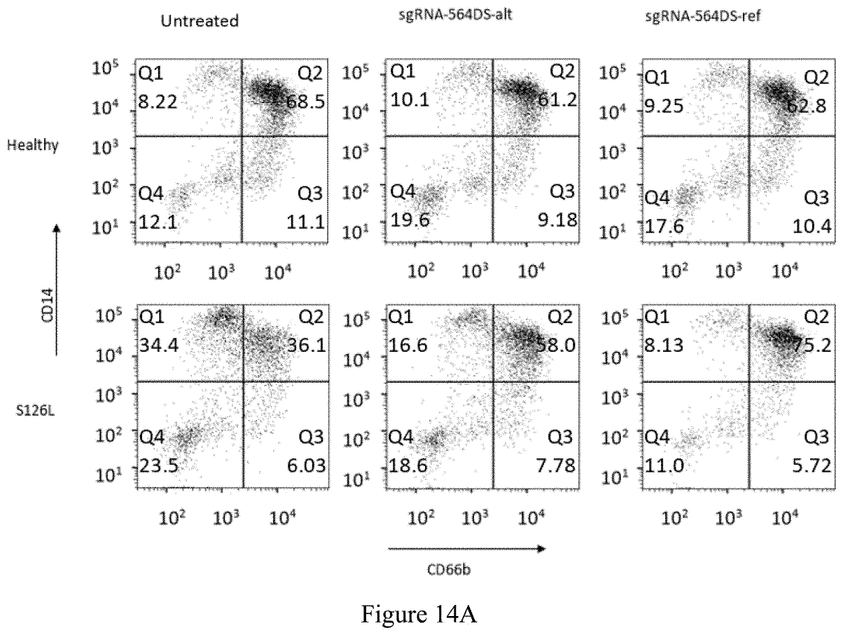

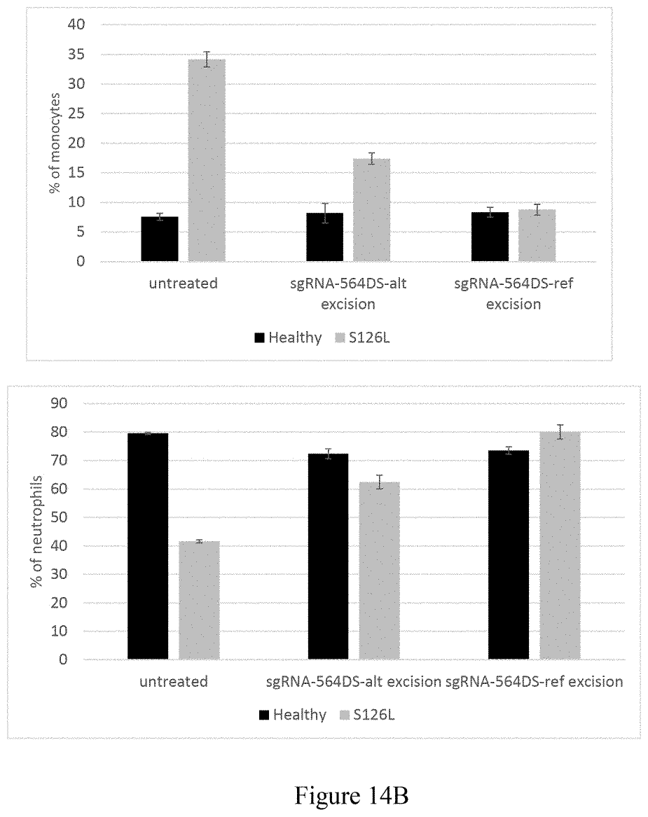

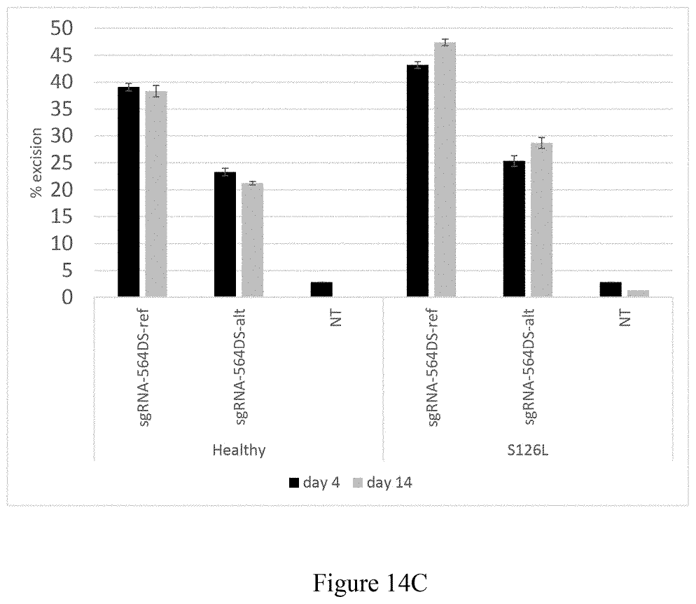

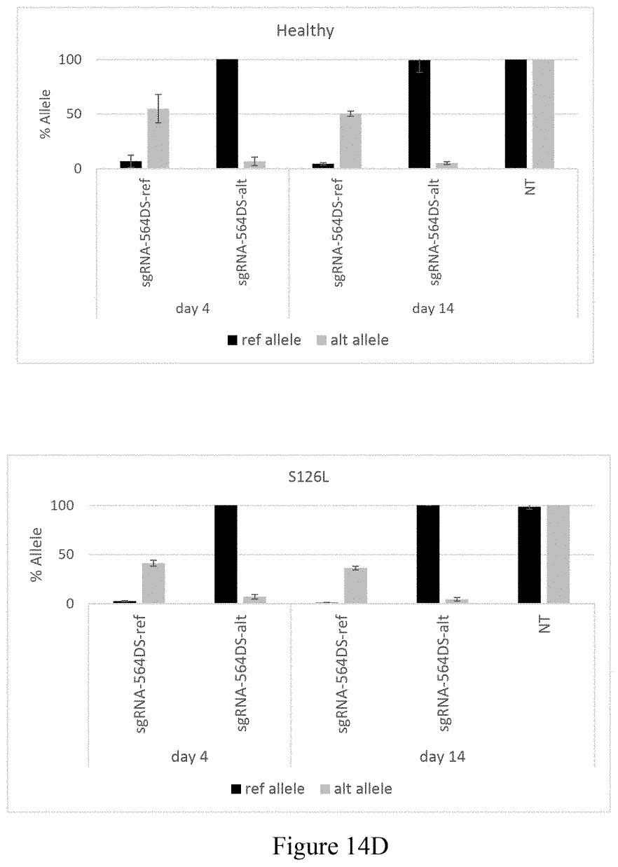

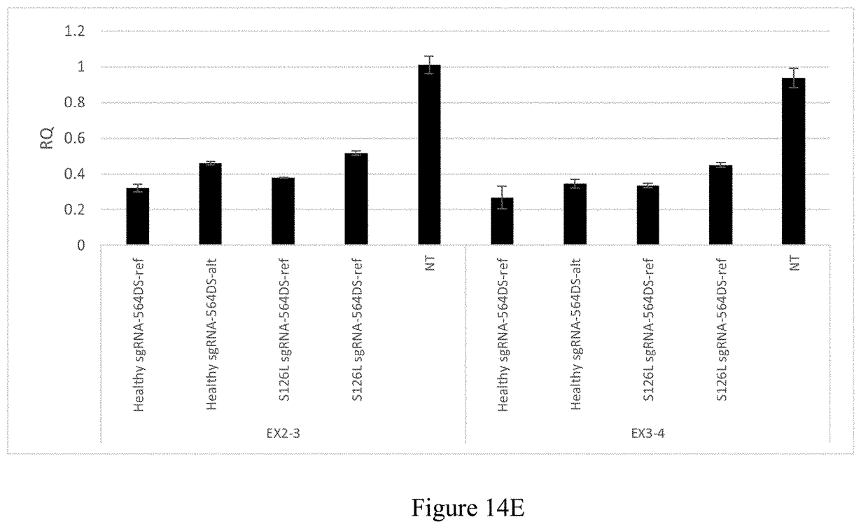

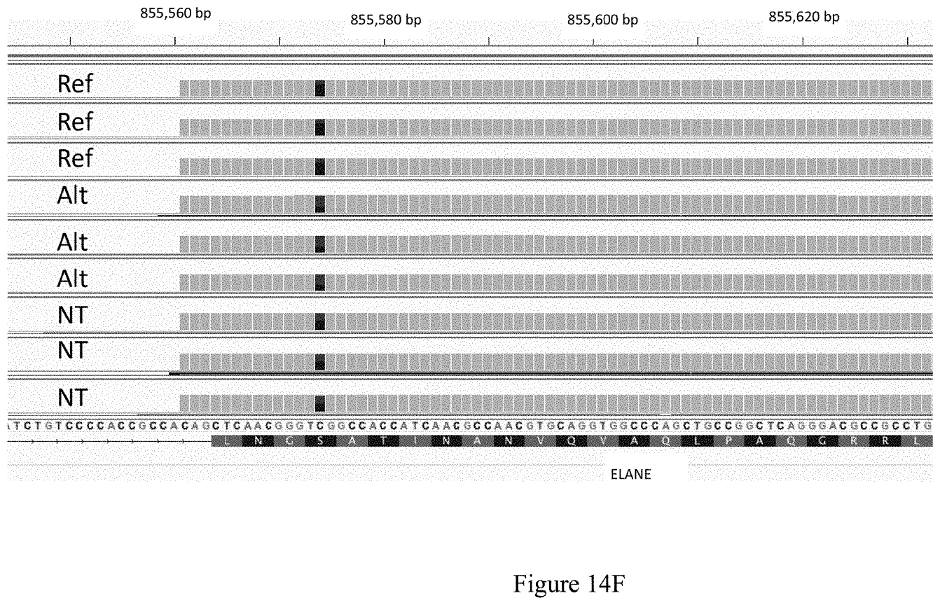

OMNI-50 guide length effect on editing efficacy was also tested in iPSCs. Cells were nucleofected with OMNI-50 RNP complexes containing guides of different spacer lengths (17-23 nt, table 4). Cells were harvested 72 h post nucleofection, gDNA was used for site specific genomic DNA amplification and capillary gel electrophoresis analysis using sg35 on-target primers, which amplify the endogenous genomic region within the target gene. The capillary gel electrophoresis data was used to determine the Total % Editing. As can be seen in FIG. 11, a spacer length of 19 nt showed levels of editing similar to background (NT), a 20 nucleotide spacer resulted in medium editing levels, and 21-23 nt spacers resulted in the most significant editing levels, in agreement with the in vitro and the U2OS experiment described above.

### Example 12: Validation of ELANE-Editing Compositions

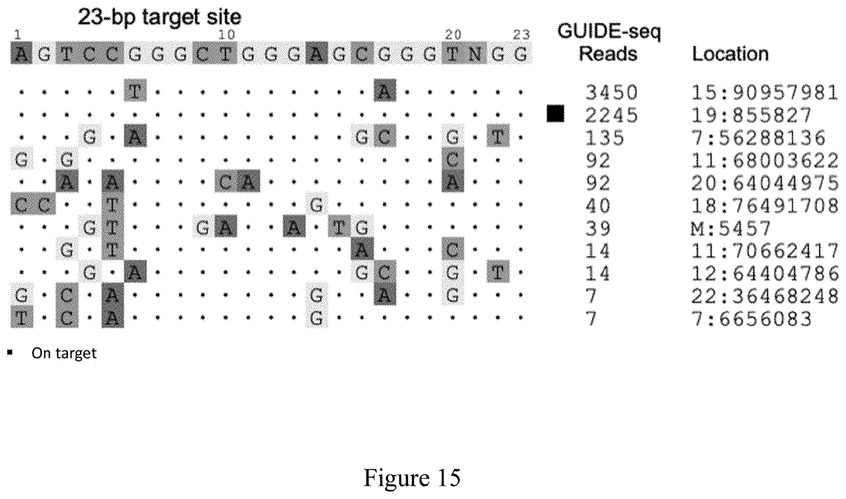

As noted above, mutations in the ELANE gene are a cause of severe congenital neutropenia (SCN), which is a predominantly autosomal dominant disorder. Hereinbelow an example of a therapeutic approach by differentially knocking out an ELANE mutant allele is presented. This can be accomplished by targeting a heterozygous rs1683564 SNP located downstream to the 3′UTR of ELANE and mediating a biallelic break in intron 4, which leads to excision of exon 5 and the 3′UTR, thereby destabilizing the transcript of the ELANE mutated allele (FIG. 15).

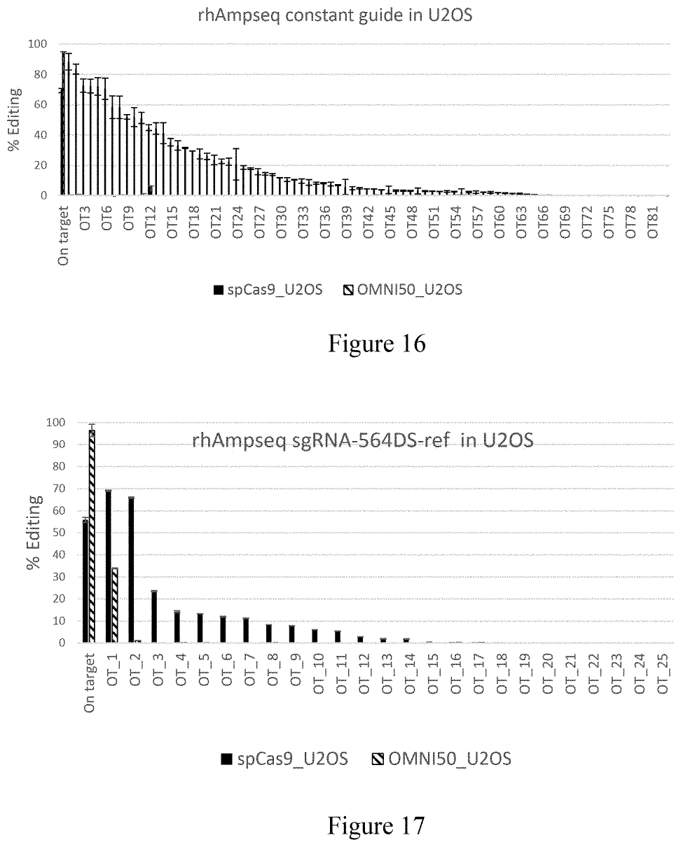

To validate the ELANE-editing composition as a treatment in patient-derived CD34+ hematopoietic stem cells (HSCs), a differentiation protocol was established. The protocol recapitulates the intrinsic defect observed in neutropenic patients by in-vitro differentiating HSCs from healthy volunteers and patients harboring ELANE mutations into neutrophils (FIG. 16A). Impaired neutrophil differentiation capacity was revealed in untreated patient derived HSCs harboring G221-termination (G221ter) or S126L ELANE mutations, displaying a 4-fold greater CD14+ monocytes and 2-fold decrease in CD66b+ neutrophils in comparison to healthy donor, consistent with the hematopoietic defect in SCN patients (FIG. 16B; FIG. 17A left panel).

To understand if the therapeutic composition rescues neutrophil differentiation and maturation defects, HSCs from a patient harboring a G221ter mutation and homozygous to the reverence form of rs1683564 was edited to excise ELANE's downstream region. This was performed by utilizing RNPs assembled from an OMNI-50 nuclease and sgRNA-564DS-ref (sg62-REF)+sgRNA constant (sg35), leading to biallelic knockout. The edited cells were then subjected to neutrophil differentiation. Excised HSCs rescued the differentiation defect leading to a 100% increase in CD14⁺ CD66b⁺ positive cells while reducing the monocytic population to normal levels in comparison to the healthy donor. Excision efficiency evaluated at day 5 and 14 of differentiation revealed a 50% enrichment of the excised population in patient cells from 30% to 45% in contrast to the healthy donor, which was consistent between both timepoints with 40% excision. This suggests a favorable therapeutic advantage for the corrected cells (FIGS. 16B-D).

Next the therapeutic composition was tested on patient HSCs harboring a S126L mutation and normal HSCs, both heterozygous for rs1683564, by utilizing RNPs assembled from OMNI-50, sgRNA-564DS-ref targeting rs1683564 reference or sgRNA-564DS-alt targeting alternative sequence, with sgRNA constant targeting the region in intron 4. The single-allele ELANE knockout composition targeting the rs1683564 reference linked to a S126L mutation resulted in deletion of the ELANE mutated allele, significantly rescued the observed differentiation abnormalities, and lead to a 100% increase in CD14⁺CD66b⁺ positive cells while reducing the monocytic population to normal. Notably, the single-allele ELANE knockout composition targeting the rs1683564 alternative resulted in deletion of the WT ELANE allele and displayed an intermediate rescue phenotype with a 50% increase in CD14⁺CD66b⁺ positive cells and 50% reduction in monocytic population (FIGS. 17A and 17B). Excision efficiency evaluated at day 4 and 14 of differentiation revealed higher efficiency with the reference allele than the alternative allele leading to ˜40% and ˜25% excision rate, respectively (FIG. 17C).

To further understand the discordance in excision efficiency between the alternative and reference guides, allele specificity of each of the guides was tested. While sgRNA-564DS-alt depicted a high degree of specificity, sgRNA-564DS-ref showed differential editing represented by the editing of mainly the reference allele and to some extent of the alternative allele as well. This explains the high degree of excision efficiency between the two guides targeting rs1683564 (FIG. 17D).

To assess the effect of the excision on the level of ELANE mRNA, total RNA was extracted from the differentiated cells and the mRNA level of ELANE was measured by qRT-PCR using primers located in regions that were not excised. The results depict more than a 50% reduction in the level of ELANE mRNA due to RNA destabilization with both reference and alternative excision compositions in normal and patient cells in comparison to untreated cells (FIG. 17E). In addition, a differential reduction in the edited alleles was determined by NGS. cDNA reads of mutated or WT ELANE allele were diminished when excising was performed with sgRNA-564DS-ref or sgRNA-564DS-alt, respectively. This result emphasizes that both excision strategies lead to reduction in the expression of ELANE in a differential manner (FIG. 17F).

TABLE 10
Guide sequence targeting rs1683564 (ref/alt), heterozygous in 42% of the
population, and non-discriminatory region (sgRNA constant).

| | Target cut site | | | Heterozygous |
|---|---|---|---|---|
| sgRNA | genomic location | Sequence | Location | Frequency |
| sgRNA- | rs1683564 | GUGUCAAGCCCC | Downstream | 42% |
| 564DS-ref | chr19:859199-859218 | AGAGGCCACA | to 3’UTR | |
| | | (SEQ ID NO: 2517) | | |
| sgRNA- | | GUGUCAAGCCCC | | |
| 564DS-alt | | AGAGGACACA | | |
| | | (SEQ ID NO: 2601) | | |
| sgRNA | chr19:855822-855844 | GCAGUCCGGGC | Intron 4 | N/A |
| constant | | UGGGAGCGGGU | | |
| | | (SEQ ID NO: 3633) | | |

These results show excision-based rescue effects neutrophil differentiation in patient-derived HSCs and demonstrate that these compositions serve as powerful SCN therapeutic.

### Example 13: Off-Target Analysis of OMNI-50 with sgRNAs Relevant to the Therapeutic Strategies of ELANE-Related SCN

To characterize the off-target activity of OMNI-50, we utilized a GUIDE-Seq methodology. This is a molecular biology technique that allows for the unbiased in vitro detection of off-target genome editing events in DNA caused by CRISPR/Cas9 as well as other RNA-guided nucleases (RGN) in living cells. Blunt-ended CRISPR RNA-guided nucleases (RGN)-induced DSBs in the genomes of living human cells are tagged by integration of a blunt double-stranded oligodeoxynucleotide (dsODN) at these breaks via an end-joining process consistent with NHEJ. dsODN integration sites in genomic DNA are precisely mapped at the nucleotide level using unbiased amplification and deep NGS.

For the study, a U2OS cell line was used and the following dsODN sequence was integrated (100 pmol): ATACCGTTATTAACATATGACAACTCAATTAAAC (SEQ ID NO: 3831). The tested guides were the ELANE constant guide and the sgRNA-564DS-ref guide.

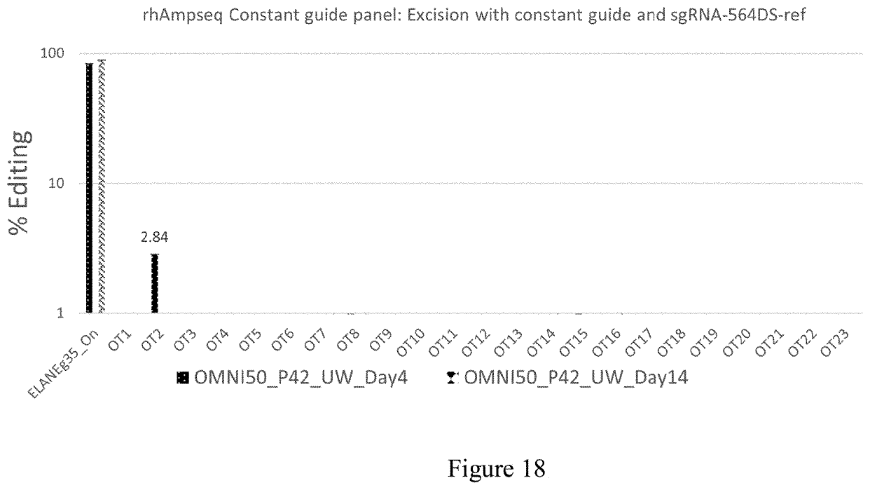

The experiment was performed with two technical repeats and sequenced using deep next generation sequencing. GUIDE-seq library preparation was performed according to Tsai et al., Nat Biotechnology, 2015 with minor modifications. The sequencing results were analyzed via “GUIDE-Seq analysis” published by Tsai et al., Nat Biotechnology (2015). Identified off-targets, sorted by GUIDE-Seq read count, are annotated in a final output table. Alignment of detected off-target sites is visualized via a color-coded sequence grid, as seen in FIG. 18.

The results from technical repeats were merged to a single list and off targets were chosen for validation via rhAmpseq technology (IDT). The rhAmpseq system enables targeted amplicon sequencing with multiplexed panels for sequencing on Illumina platforms.

For generation of rhAmpseq panels, the following filters were applied on the final off-target list generated by GUIDE-seq analysis:

TABLE 11
Reads filtering criteria for generation of rhAmpSeq panels

| | OT in | | |
|---|---|---|---|
| | gene | Reads | Reads |
| Nuclease | (Yes/No) | sample A | sample B |
| SpCas9 | Yes | ≥100 | ≥100 |
| SpCas9 | No | ≥500 | ≥500 |

| | | |
|---|---|---|
| OMNI-50 | Yes | No Filter |
| OMNI-50 | No | No Filter |

The final panels had the following off targets:

TABLE 12
Number of off targets in each panel

| | Number of |
|---|---|
| Guide | Off targets |
| Constant guide | 110 |
| sgRNA-564DS-ref | 26 |
| sgRNA-564DS-Alt | 23 |

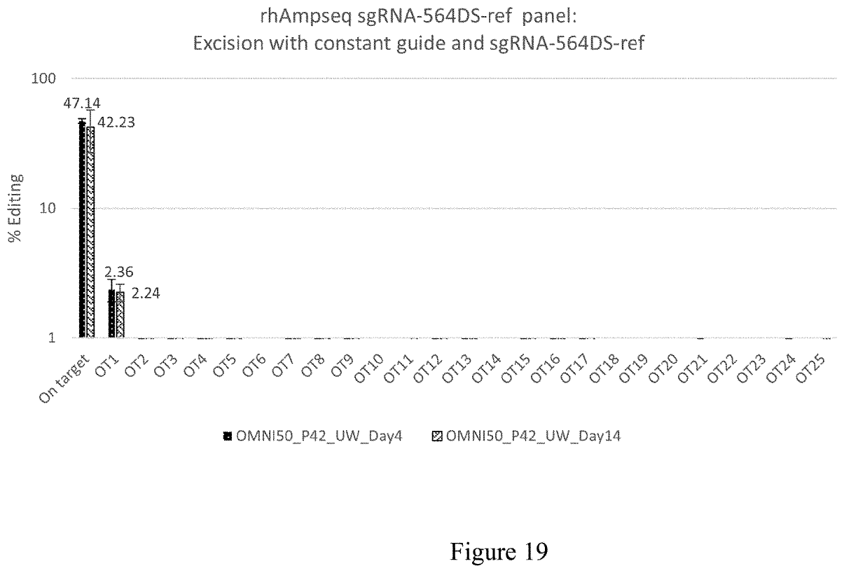

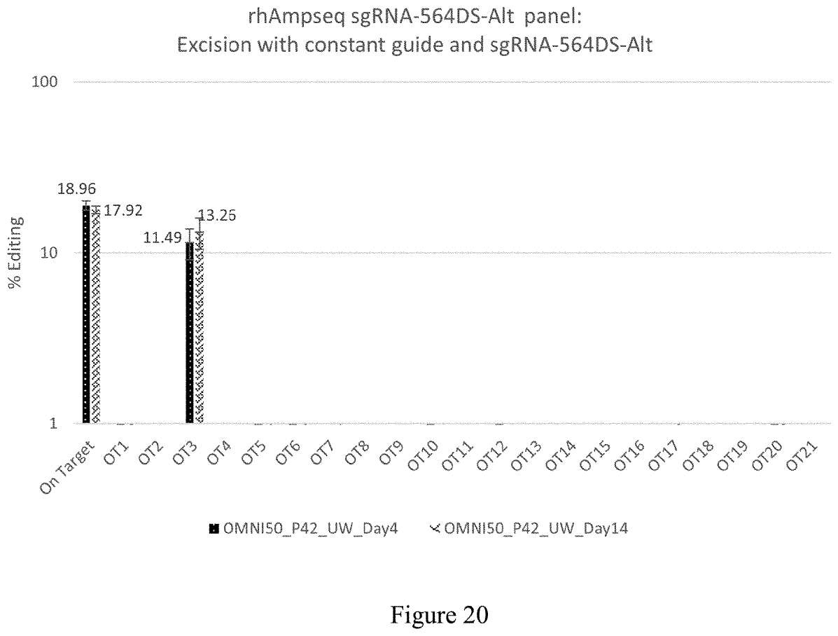

For validation of the off-targets, an IDT rhAmpseq protocol was used with the panels generated for the ELANE constant guide and the sgRNA-564DS-ref guide. U2OS cells were edited with the mentioned RNA guides either with SpCas9 or OMNI-50 nucleases (FIG. 19 and FIG. 20). The rhAmpseq libraries were sequenced by NextSeq PE150 sequencing and analyzed with an IDT pipeline.

The results described in FIGS. 19 and 20 indicate that OMNI-50 displays high on-target activity and specificity, relative to SpCas9. Out of 110 off-targets that were analyzed for sgConstant, OMNI-50 had an editing activity of >0.5% on only one site (OT-2), compared to more than 60 sites with SpCas9. In the case of sgRNA-564DS-Ref, although OMNI-50 had higher on target activity than SpCas9, only 2 out of 25 off-target sites showed editing levels higher than 0.5% as opposed to SpCas9 which had 14 sites with a % of editing value higher than 0.5%.

In addition, we validated the off-target panels on patient-derived HSCs treated with either the constant guide and the sgRNA-564DS-ref or with the constant guide and the sgRNA-564DS-Alt guide (See example as described for FIG. 17). This leads to excision of exon 5 and the 3′UTR, as described in FIG. 15. The results showed no validated off-targets for constant guide out of 110 targets that were examined (FIG. 21). For the sgRNA-564DS-ref guide and the sgRNA-564DS-Alt guide, one off-target to each panel was validated (FIG. 22 and FIG. 23). These results emphasize the relative high fidelity of OMNI-50 nuclease.

### REFERENCES

1. Ahmad and Allen (1992) “Antibody-mediated Specific Binging and Cytotoxicity of Lipsome-entrapped Doxorubicin to Lung Cancer Cells in Vitro”, Cancer Research 52:4817-20
2. Anders (1992) “Human gene therapy”, Science 256:808-13
3. Basha et al. (2011) “Influence of Cationic Lipid Composition on Gene Silencing Properties of Lipid Nanoparticle Formulations of siRNA in Antigen-Presenting Cells”, Mol. Ther. 19(12):2186-200
4. Behr (1994) Gene transfer with synthetic cationic amphiphiles: Prospects for gene therapy”, Bioconjuage Chem 5:382-89
5. Blaese (1995) “Vectors in cancer therapy: how will they deliver”, Cancer Gene Ther.

2:291-97
6. Blaese et al. (1995) “T lympocyte-directed gene therapy for ADA-SCID: initial trial results after 4 years”, Science 270(5235):475-80
7. Boxer, L. A. (2012) “How to approach neutropenia”, Hematology Am Soc Hematol Educ

Program 2012: 174-182
8. Buchschacher and Panganiban (1992) “Human immunodeficiency virus vectors for inducible expression of foreign genes”, J. Virol. 66:2731-39
9. Burstein et al. (2017) “New CRISPR-Cas systems from uncultivated microbes”, Nature 542:237-41
10. Carlsson, G et al. (2012) “Incidence of severe congenital neutropenia in Sweden and risk of evolution to myelodysplastic syndrome/leukaemia”, Br J Haematol 158(3): 363-369
11. Chung et al. (2006) “Agrobacterium is not alone: gene transfer to plants by viruses and other bacteria”, Trends Plant Sci. 11(1):1-4
12. Connelly, J. A. et al. (2012) “Hematopoietic stem cell transplantation for severe congenital neutropenia”, Curr Opin Hematol 19(1): 44-51
13. Crystal (1995) “Transfer of genes to humans: early lessons and obstacles to success”,

Science 270(5235):404-10
14. Dale, D. C. (2017) “How I manage children with neutropenia”, Br J Haematol 178(3): 351-363
15. Dillon (1993) “Regulation gene expression in gene therapy” Trends in Biotechnology 11(5):167-173
16. Donadieu, J. et al. (2011) “Congenital neutropenia: diagnosis, molecular bases and patient management”, Orphanet J Rare Dis 6: 26.
17. Dranoff et al. (1997) “A phase I study of vaccination with autologous, irradiated melanoma cells engineered to secrete human granulocyte macrophage colony stimulating factor”, Hum. Gene Ther. 8(1):111-23
18. Donadieu et al. (2011) “Congenital neutropenia: diagnosis, molecular bases and patient management”, Orphanet J Rare Dis. 6:26
19. Dunbar et al. (1995) “Retrovirally marked CD34-enriched peripheral blood and bone marrow cells contribute to long-term engraftment after autologous transplantation”, Blood 85:3048-57
20. Ellem et al. (1997) “A case report: immune responses and clinical course of the first human use of ganulocyte/macrophage-colony-stimulating-factor-tranduced autologous melanoma cells for immunotherapy”, Cancer Immunol Immunother 44:10-20
21. Gao and Huang (1995) “Cationic liposome-mediated gene transfer” Gene Ther. 2(10):710-22
22. Germeshausen et al. (2013) “The spectrum of ELANE mutations and their implications in severe congenital and cyclic neutropenia”, Hum Mutat. 34(6):905-14
23. Haddada et al. (1995) “Gene Therapy Using Adenovirus Vectors”, in: The Molecular Repertoire of Adenoviruses III: Biology and Pathogenesis, ed. Doerfler and Bohm, pp. 297-306
24. Han et al. (1995) “Ligand-directed retro-viral targeting of human breast cancer cells”, Proc Natl Acad Sci U.S.A. 92(21):9747-51
25. Horwitz, M. S. et al. (2013) “ELANE mutations in cyclic and severe congenital neutropenia: genetics and pathophysiology”, Hematol Oncol Clin North Am 27(1): 19-41, vii.
26. Inaba et al. (1992) “Generation of large numbers of dendritic cells from mouse bone marrow cultures supplemented with granulocyte/macrophage colony-stimulating factor”, J Exp Med. 176(6):1693-702
27. Jinek et al. (2012) “A programmable dual-RNA-guided DNA endonuclease in adaptive bacterial immunity,” Science 337(6096):816-21
28. Johan et al. (1992) “GLVR1, a receptor for gibbon ape leukemia virus, is homologous to a phosphate permease of Neurospora crassa and is expressed at high levels in the brain and thymus”, J Virol 66(3):1635-40
29. Judge et al. (2006) “Design of noninflammatory synthetic siRNA mediating potent gene silencing in vivo”, Mol Ther. 13(3):494-505
30. Kim, E. et al. In vivo genome editing with a small Cas9 orthologue derived from Campylobacter jejuni. Nat. Commun. 8, 14500 (2017).
31. Kohn et al. (1995) “Engraftment of gene-modified umbilical cord blood cells in neonates with adnosine deaminase deficiency”, Nature Medicine 1:1017-23
32. Koonen et al. (2017) “Diversity, classification and evolution of CRIPR-Cas systems”, Current Opinion in Microbiology 37:67-78
33. Kremer and Perricaudet (1995) “Adenovirus and adeno-associated virus mediated gene transfer”, Br. Med. Bull. 51(1):31-44
34. Macdiarmid et al. (2009) “Sequential treatment of drug-resistant tumors with targeted minicells containing siRNA or a cytotoxic drug”, Nat Biotehcnol. 27(7):643-51
35. Malech et al. (1997) “Prolonged production of NADPH oxidase-corrected granulocyes after gene therapy of chronic granulomatous disease”, PNAS 94(22):12133-38
36. Makaran et al. (2015) “The diversity of mutations and clinical outcomes for ELANE-associated neutropenia”, Curr Opin Hematol. 22(1):3-11
37. Miller et al. (1991) “Construction and properties of retrovirus packaging cells based on gibbon ape leukemia virus”, J Virol. 65(5):2220-24
38. Miller (1992) “Human gene therapy comes of age”, Nature 357:455-60
39. Mitani and Caskey (1993) “Delivering therapeutic genes—matching approach and application”, Trends in Biotechnology 11(5):162-66
40. Mojica, F. J., et al. (1993) “Direct gene transfer for immunotherapy and immunization”,

Trends in Biotechnology 11(5):211-15
41. Remy et al. (1994) “Gene Transfer with a Series of Lipphilic DNA-Binding Molecules”,

Bioconjugate Chem. 5(6):647-54
42. Rosenberg, P. S. et al. (2010) “Stable long-term risk of leukaemia in patients with severe congenital neutropenia maintained on G-CSF therapy”, Br J Haematol 150(2): 196-199.
43. Schaffer, A et al. (2007) “Genetic heterogeneity in severe congenital neutropenia: how many aberrant pathways can kill a neutrophil?” Curr Opin Allergy Clin Immunol 7(6): 481-494
44. Sentmanat et al. (2018) “A Survey of Validation Strategies for CRISPR-Cas9 Editing”, Scientific Reports 8:888, doi:10.1038/s41598-018-19441-8
45. Skokowa, J. et al. (2017) “Severe congenital neutropenias”, Nat Rev Dis Primers 3: 17032
46. Sommerfelt et al. (1990) “Localization of the receptor gene for type D simian retroviruses on human chromosome 19”, J. Virol. 64(12):6214-20
47. Tsai et al., (2015) “GUIDE-Seq analysis” Nat Biotechnology 33, 187-197
48. Van Brunt (1988) “Molecular framing: transgenic animals as bioactors” Biotechnology 6:1149-54
49. Vigne et al. (1995) “Third-generation adenovectors for gene therapy”, Restorative Neurology and Neuroscience 8(1,2): 35-36
50. Wilson et al. (1989) “Formation of infectious hybrid virion with gibbon ape leukemia virus and human T-cell leukemia virus retroviral envelope glycoproteins and the gag and pol proteins of Moloney murine leukemia virus”, J. Virol. 63:2374-78
51. Yu et al. (1994) “Progress towards gene therapy for HIV infection”, Gene Ther. 1(1):13-26
52. Yu, K et al. (2016) “Gene Editing of Human Hematopoietic Stem and Progenitor Cells: Promise and Potential Hurdles”, Hum Gene Ther 27(10): 729-740
53. Zetsche et al. (2015) “Cpf1 is a single RNA-guided endonuclease of a class 2 CRIPSR-Cas system” Cell 163(3):759-71
54. Zhenwang et al. (2017) “Large-scale ex vivo generation of human neutrophils from cord blood CD34⁺ cells”, PLOS ONE, available at <doi.org/10.1371/journal.pone.0180832>
55. Zuris et al. (2015) “Cationic lipid-mediated delivery of proteins enables efficient protein based genome editing in vitro and in vivo” Nat Biotechnol. 33(1):73-80

1. A method for inactivating in a cell a mutant allele of the elastase, neutrophil expressed gene (ELANE gene) gene having a mutation associated with severe congenital neutropenia (SCN) or cyclic neutropenia (CyN) and which cell is heterozygous at one or more polymorphic sites selected from the group consisting of: rs1683564, rs10424470, rs4807932, rs10414837, rs376107533, rs3761010, rs351108, rs3826946, rs10413889, rs3761007, rs10409474, rs3761005, rs351107, rs3761001, rs740021, rs781452480, rs371057361, rs570466264, rs1041904080, rs7250194, rs17216649, rs199720952, rs6510983, rs17223066, rs7255385, rs9749274, rs111361200, rs112639467, rs141213775, rs28591229, rs10469327, rs3834645,
rs71335276, and rs8107095, the method comprising introducing to the cell a composition comprising:
a CRISPR nuclease or a sequence encoding the CRISPR nuclease; and
a first RNA molecule comprising a guide sequence portion having 21-30 nucleotides,
wherein a complex of the CRISPR nuclease and the first RNA molecule affects a double strand break in the mutant allele of the ELANE gene.

2. The method of claim 1, wherein the guide sequence portion of the first RNA molecule comprises 21-30 contiguous nucleotides containing nucleotides in the sequence as set forth in any one of SEQ ID NOs: 2517, 2601, 2464-2516, 2518-2600, 2602-2613, 1-2463, 2614-3751 or SEQ ID NOs: 1-2953, or
wherein the cell is heterozygous at one or more polymorphic sites selected from the group consisting of: rs1683564, rs9749274, rs740021, rs199720952, rs28591229, rs71335276, rs3826946, rs10413889, rs3761005, rs10409474, rs3761007, rs17216649, rs10469327, rs8107095, rs10414837, and rs10424470, or
wherein the polymorphic site is rs1683564 and the guide sequence portion of the first RNA molecule comprises 21-30 contiguous nucleotides containing nucleotides in the sequence as set forth in any one of SEQ ID NOs: 2517, 2601, 2464-2516, 2518-2600, 2602-2613, 1-2463, 2614-3751, and/or
wherein the complex of the CRISPR nuclease and the first RNA molecule affects a double strand break in the mutant allele of the ELANE gene, which mutant allele is targeted for the double strand break based on the one or more polymorphic sites.

3. (canceled)

4. (canceled)

5. (canceled)

6. The method of claim 1, further comprising introduction of a second RNA molecule comprising a guide sequence portion capable of complexing with a CRISPR nuclease, wherein the complex of the second RNA molecule and CRISPR nuclease affects a second double strand break in the ELANE gene.

7. The method of claim 6, wherein the second double strand break is within a non-coding region of the ELANE gene, or
wherein the non-coding region of the ELANE gene is intron 4, and/or
wherein the guide sequence portion of the second RNA molecule comprises 21-30 contiguous nucleotides containing nucleotides in the sequence as set forth in any one of SEQ ID NOs: 2954-3751, or
wherein the cell is heterozygous at rs10414837 or rs3761005 and wherein the complex of the second RNA molecule and CRISPR nuclease affects a double strand break in intron 4 of the ELANE gene, or
wherein the cell is heterozygous at rs1683564 and wherein the complex of the second RNA molecule and CRISPR nuclease affects a double strand break in intron 4 of the ELANE gene, and/or
wherein the second RNA molecule comprises a guide sequence portion having 21-30 nucleotides.

8. (canceled)

9. (canceled)

10. (canceled)

11. (canceled)

12. (canceled)

13. The method of claim 1, comprising obtaining the cell with an ELANE gene mutation associated with severe congenital neutropenia (SCN) or CyN from a subject with an ELANE gene mutation related to SCN or CyN and/or suffering from SCN or CyN and which subject is heterozygous at one or more polymorphic sites selected from the group consisting of: rs10424470, rs4807932, rs10414837, rs376107533, rs3761010, rs351108, rs3826946, rs10413889, rs3761007, rs10409474, rs3761005, rs351107, rs3761001, rs740021, rs781452480, rs371057361, rs570466264, rs1041904080, rs7250194, rs17216649, rs199720952, rs6510983, rs17223066, rs7255385, rs9749274, rs111361200, rs112639467, rs141213775, rs28591229, rs10469327, rs3834645, rs1683564, rs71335276, and rs8107095, preferably obtaining the cell from the subject by mobilization and/or by apheresis, or by bone marrow aspiration.

14. (canceled)

15. (canceled)

16. (canceled)

17. The method of claim 1, wherein the cell is prestimulated prior to introducing the composition to the cell.

18. The method of claim 13 further comprising culture expanding the cell to obtain cells preferably wherein the cells are cultured with one or more of: stem cell factor (SCF), IL-3, and GM-CSF, and/or
wherein the cells are cultured with at least one cytokine, preferably wherein the at least one cytokine is a recombinant human cytokine.

19. (canceled)

20. (canceled)

21. (canceled)

22. (canceled)

23. The method of claim 1, wherein introducing the composition comprising the first RNA molecule or introduction of the second RNA molecule comprises electroporation of the cell or cells.

24. A modified cell obtained by the method of claim 1, wherein the cell is among a plurality of cells, wherein the composition comprising the first RNA molecule is introduced into at least the cell as well as other cells among the plurality of cells, and the mutant allele of the ELANE gene is inactivated in at least the cell as well as in the other cells among the plurality of cells, thereby obtaining multiple modified cells.

25. (canceled)

26. The modified cell or cells of claim 24, wherein the cell, or cells obtained from culture expanding the cell, are capable of
engraftment;
giving rise to progeny cells;
giving rise to progeny cells after engraftment;
giving rise to progeny cells after an autologous engraftment; or
giving rise to progeny cells for at least 12 months or at least 24 months after engraftment.

27. (canceled)

28. (canceled)

29. (canceled)

30. (canceled)

31. The modified cell or cells claim 24, wherein the modified cell or cells are hematopoietic stem cells and/or progenitor cells (HSPCs);
wherein the modified cell or cells are CD34+ hematopoietic stem cells; or
wherein the modified cell or cells are bone marrow cells or peripheral mononucleated cells (PMCs).

32. (canceled)

33. (canceled)

34. (canceled)

35. (canceled)

36. A composition comprising the modified cells of claim 24 and a pharmaceutically acceptable carrier.

37. (canceled)

38. A method of preparing in vitro or ex vivo a composition comprising modified cells, the method comprising:
(a) isolating HSPCs from cells obtained from a subject with an ELANE gene mutation related to SCN or CyN and/or suffering from SCN or CyN and which subject is heterozygous at one or more polymorphic sites selected from the group consisting of rs10424470, rs4807932, rs10414837, rs376107533, rs3761010, rs351108, rs3826946, rs10413889, rs3761007, rs10409474, rs3761005, rs351107, rs3761001, rs740021, rs781452480, rs371057361, rs570466264, rs1041904080, rs7250194, rs17216649, rs199720952, rs6510983, rs17223066, rs7255385, rs9749274, rs111361200, rs112639467, rs141213775, rs28591229, rs10469327, rs3834645, rs1683564, rs71335276, and rs8107095, and obtaining the cell from the subject;
(b) introducing to the cells of step (a) a composition comprising:
a CRISPR nuclease or a sequence encoding the CRISPR nuclease; and
a first RNA molecule comprising a guide sequence portion having 21-30 nucleotides,
wherein a complex of the CRISPR nuclease and the first RNA molecule affects a double strand break in the mutant allele of the ELANE gene in one or more cells,
optionally, introducing to the cells a second RNA molecule comprising a guide sequence portion capable of complexing with a CRISPR nuclease, wherein the complex of the second RNA molecule and CRISPR nuclease affects a second double strand break in the ELANE gene in the one or more cells
so as to inactivate the mutant allele of the ELANE gene in one or more cells thereby obtaining modified cells; optionally
(c) culture expanding the modified cells of step (b),
wherein the modified cells are capable of engraftment and giving rise to progeny cells after engraftment.

39. (canceled)

40. A method of treating a subject afflicted with SCN or CyN, comprising administration of a therapeutically effective amount of the composition of claim 36, or
treating SCN or CyN in a subject with an ELANE gene mutation relating to SCN or CYN in need thereof and which subject is heterozygous at one or more polymorphic sites selected from the group consisting of: rs10424470, rs4807932, rs10414837, rs376107533, rs3761010, rs351108, rs3826946, rs10413889, rs3761007, rs10409474, rs3761005, rs351107, rs3761001, rs740021, rs781452480, rs371057361, rs570466264, rs1041904080, rs7250194, rs17216649, rs199720952, rs6510983, rs17223066, rs7255385, rs9749274, rs111361200, rs112639467, rs141213775, rs28591229, rs10469327, rs3834645, rs1683564, rs71335276, and rs8107095, the method comprising:
(a) isolating HSPCs from cells obtained from the subject;
(b) introducing to the cells of step (a) a composition comprising:
a CRISPR nuclease or a sequence encoding the CRISPR nuclease; and
a first RNA molecule comprising a guide sequence portion having 21-30 nucleotides,
wherein a complex of the CRISPR nuclease and the first RNA molecule affects a double strand break in the mutant allele of the ELANE gene in one or more cells,
optionally, introducing to the cells a second RNA molecule comprising a guide sequence portion capable of complexing with a CRISPR nuclease, wherein the complex of the second RNA molecule and CRISPR nuclease affects a second double strand break in the ELANE gene in the one or more cells
so as to inactivate the mutant allele of the ELANE gene in one or more cells thereby obtaining modified cells;
optionally;
(c) culture expanding the cells of step (b) wherein the modified cells are capable of engraftment and giving rise to progeny cells after engraftment; and
(d) administering to the subject the cells of step (b) or step (c)
thereby treating the SCN or CyN in the subject, or
which subject is heterozygous at one or more polymorphic sites selected from the group consisting of: rs7250194, rs397773837, rs759823713, rs12976041, rs55921706, rs2007647, rs7255385, rs351108, rs6510983, rs375312008, rs1234492733, rs8111201, rs3761010, rs11881698, rs17223066, rs74176357, rs201984870, rs141213775, rs111361200, rs3761001, rs55793725, rs55791968, rs953484068, rs56234325, rs4807932, rs9304953, rs71335273, rs868711974, and rs570466264, the method comprising
administering to the subject autologous modified cells or progeny of autologous modified cells, wherein the autologous modified cells are modified so as to have a double strand break in the mutant allele of the ELANE gene,
wherein said double strand break results from introduction to the cells of a composition comprising a CRISPR nuclease or sequence encoding the CRISPR nuclease and a first RNA molecule wherein a complex of the CRISPR nuclease and the first RNA molecule affects a double strand break in the mutant allele of the ELANE gene so as to inactive the mutant allele of the ELANE gene in the cell,
thereby treating the SCN or CyN in the subject.

41. (canceled)

42. (canceled)

43. (canceled)

44. An RNA molecule comprising a guide sequence portion having 21-30 contiguous nucleotides containing nucleotides in the sequence set forth in any one of SEQ ID NOs: 1-3751, or composition comprising the RNA and a second RNA molecule comprising a guide sequence portion, optionally wherein the guide sequence portion of the second RNA molecule has 21-30 contiguous nucleotides containing nucleotides in the sequence set forth in any of SEQ ID NOs: 2954-3751,
wherein the second RNA molecule targets a non-coding region of the ELANE gene;
wherein the nucleotide sequence of the guide sequence portion of the second RNA molecule is a different nucleotide sequence from the sequence of the guide sequence portion of the first RNA molecule,
wherein the first and/or second RNA molecule further comprises a portion having a sequence which binds to a CRISPR nuclease;
wherein the sequence which binds to a CRISPR nuclease is a tracrRNA sequence;
wherein the first and/or second RNA molecule further comprises a portion having a tracr mate sequence;
wherein the second RNA molecule further comprising one or more linker portions; and/or
wherein the first and/or second RNA molecule is up to 300 nucleotides in length; and/or
the composition further comprising one or more CRISPR nucleases or sequences encoding the one or more CRISPR nucleases, and/or one or more tracrRNA molecules or sequences encoding the one or more tracrRNA molecules.

45. (canceled)

46. (canceled)

47. (canceled)

48. (canceled)

49. (canceled)

50. (canceled)

51. (canceled)

52. (canceled)

53. (canceled)

54. A method for inactivating in a cell a mutant ELANE allele, the method comprising delivering to the cell the RNA molecule or composition of claim 44
preferably wherein the method comprises:
(a) removing an exon containing a disease-causing mutation from a mutant allele, wherein the first RNA molecule or the first and the second RNA molecules target regions flanking an entire exon or a portion of the exon;
(b) removing multiple exons, the entire open reading frame of a gene, or removing the entire gene;
(c) the first RNA molecule or the first and the second RNA molecules targeting an alternative splicing signal sequence between an exon and an intron of a mutant allele;
(d) the second RNA molecule targeting a sequence present in both a mutant allele and a functional allele;
(e) the second RNA molecule targeting an intron; or
(f) subjecting the mutant allele to insertion or deletion by an error prone non-homologous end joining (NHEJ) mechanism, generating a frameshift in the mutant allele's sequence,
optionally wherein the frameshift results in inactivation or knockout of the mutant allele
preferably wherein, the frameshift creates an early stop codon in the mutant allele or the frameshift results in nonsense-mediated mRNA decay of the transcript of the mutant allele; or
wherein the inactivating results in a truncated protein encoded by the mutant allele and a functional protein encoded by the functional allele; and/or wherein
(a) the cells or the subject is heterozygous at rs10414837 or rs3761005 and wherein the complex of the second RNA molecule and CRISPR nuclease affects a double strand break in intron 4 of the ELANE gene; or
(b) the cells or the subject is heterozygous at rs1683564 and wherein the complex of the second RNA molecule and CRISPR nuclease affects a double strand break in intron 4 of the ELANE gene; and/or
wherein the one or more CRISPR nuclease and/or the tracrRNA and the RNA molecule or RNA molecules are delivered to the subject and/or cells substantially at the same time or at different times.

55. A method for treating SCN or CyN, the method comprising delivering to a subject having SCN or CyN the RNA molecule or composition of claim 44
preferably wherein the method comprises:
(a) removing an exon containing a disease-causing mutation from a mutant allele, wherein the first RNA molecule or the first and the second RNA molecules target regions flanking an entire exon or a portion of the exon;
(b) removing multiple exons, the entire open reading frame of a gene, or removing the entire gene;
(c) the first RNA molecule or the first and the second RNA molecules targeting an alternative splicing signal sequence between an exon and an intron of a mutant allele;
(d) the second RNA molecule targeting a sequence present in both a mutant allele and a functional allele;
(e) the second RNA molecule targeting an intron; or
(f) subjecting the mutant allele to insertion or deletion by an error prone non-homologous end joining (NHEJ) mechanism, generating a frameshift in the mutant allele's sequence,
optionally wherein the frameshift results in inactivation or knockout of the mutant allele
preferably wherein, the frameshift creates an early stop codon in the mutant allele or the frameshift results in nonsense-mediated mRNA decay of the transcript of the mutant allele; or
wherein the treating results in a truncated protein encoded by the mutant allele and a functional protein encoded by the functional allele; and/or wherein
(a) the cells or the subject is heterozygous at rs10414837 or rs3761005 and wherein the complex of the second RNA molecule and CRISPR nuclease affects a double strand break in intron 4 of the ELANE gene; or
(b) the cells or the subject is heterozygous at rs1683564 and wherein the complex of the second RNA molecule and CRISPR nuclease affects a double strand break in intron 4 of the ELANE gene; and/or
wherein the one or more CRISPR nuclease and/or the tracrRNA and the RNA molecule or RNA molecules are delivered to the subject and/or cells substantially at the same time or at different times.

56. (canceled)

57. (canceled)

58. (canceled)

59. (canceled)

60. (canceled)

61. (canceled)

62. (canceled)

63. (canceled)

64. A kit for inactivating a mutant ELANE allele in a cell, comprising the RNA molecule of claim 44, a CRISPR nuclease or a sequence encoding the CRISPR nuclease, and/or a tracrRNA molecule or a sequence encoding the tracrRNA; and instructions for delivering the RNA molecule; CRISPR nuclease or a sequence encoding the CRISPR nuclease, and/or the tracrRNA molecule or a sequence encoding the tracrRNA to the cell to inactivate the mutant ELANE allele in the cell; or
a kit for treating SCN or CyN in a subject, comprising the RNA molecule claim 44, a CRISPR nuclease or a sequence encoding the CRISPR nuclease, and/or a tracrRNA molecule or a sequence encoding the tracrRNA; and instructions for delivering the RNA molecule; CRISPR nuclease or sequence encoding the CRISPR nuclease, and/or tracrRNA molecule or sequence encoding the tracrRNA to a subject having or at risk of having SCN or CyN so as to treat the SCN or CyN; or
a kit for inactivating a mutant ELANE allele in a cell, comprising the composition of claim 36, and instructions for delivering the composition to the cell so as to inactivate the ELANE gene in the cell; or
a kit for treating SCN or CyN in a subject, comprising the composition of claim 36, and instructions for delivering the composition to a subject having or at risk of having SCN or CyN so as to treat SCN or CyN.

65. (canceled)

66. (canceled)

67. (canceled)

68. The method of claim 1, wherein the CRISPR nuclease has cleavage activity when used with an RNA molecule comprising a guide sequence portion having 21-30 nucleotides, and/or
wherein the CRISPR nuclease is derived from any of Streptococcus pyogenes, Streptococcus thermophilus, Streptococcus sp., Staphylococcus aureus, Neisseria meningitidis, Treponema denticola, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Francisella cf. novicida Fx1, Alicyclobacillus acidoterrestris, Oleiphilus sp., Bacterium CGO9_39_24, and Deltaproteobacteria bacterium; and/or
wherein the CRISPR nuclease has one or more of the following features:
(a) greater cleavage activity when used with an RNA molecule comprising a guide sequence portion having 21-23 nucleotides, compared to its cleavage activity when used with an RNA molecule comprising a guide sequence portion having 20 or fewer nucleotides, and/or 24 or more nucleotides;
(b) greater cleavage activity when used with an RNA molecule comprising a guide sequence portion having 21-22 nucleotides, compared to its cleavage activity when used with an RNA molecule comprising a guide sequence portion having 20 or fewer nucleotides, and/or 23 or more nucleotides; and
(c) greatest cleavage activity when used with an RNA molecule comprising a guide sequence portion having 22 nucleotides; and/or
wherein the CRISPR nuclease has at least 95% identity to the amino acid sequence as set forth in SEQ ID NO:3789 or the sequence encoding the CRISPR nuclease has at least a 95% sequence identity to SEQ ID NO:3790 or SEQ ID NO:3791; and/or
wherein the composition comprising the CRISPR nuclease has one or more of the following features:
(a) the composition is in an aqueous solution;
(b) the pH of the composition is between 6 and 8;
(c) the composition is free of RNase; and/or
wherein the first RNA molecule comprises a guide sequence portion having 21-22 nucleotides.

69. (canceled)

70. (canceled)

71. (canceled)

72. (canceled)

73. (canceled)

74. The RNA molecule of claim 44, wherein the guide sequence portion has 21-22 contiguous nucleotides in a sequence set forth in any one of SEQ ID NOs: 1-3751.
